(19) United States
(12) Patent Application Publication
(10) Pub. No.: US 2012/0283216 A1
(43) Pub. Date: Nov. 8, 2012

(54) FUNGICIDAL 2-(BICYCLIC ARYLOXY)CARBOXAMIDES

(76) Inventors: Alvin Donald Crews, JR., Voorhees, NJ (US); Amy X. Ding, Wilmington, DE (US); Chi-Ping Tseng, Wilmington, DE (US)

(21) Appl. No.: 13/513,056

(22) PCT Filed: Dec. 22, 2010
(86) PCT No.: PCT/US10/61765

§ 371 Date: May 31, 2012

(30) Foreign Application Priority Data
Dec. 22, 2009 (US) 61289000

(51) Int. Cl. A01N 37/18; A01N 37/34; C07C 235/06; C07C 243/28; A01N 37/28; C07F 9/32; A01N 57/22; C07C 271/20; A01N 47/12; C07C 327/30; C07F 7/10; A01N 55/10; C07D 215/20; A01N 43/42; C07D 217/24; A01P 3/00; C07C 255/29

(52) U.S. Cl. 514/63; 558/393; 514/510; 564/172; 514/622; 564/150; 514/615; 558/174; 514/119; 560/28; 514/479; 514/481; 558/254; 556/419; 546/153; 514/312; 546/14; 546/141; 514/309

(57) ABSTRACT

Disclosed are compounds of Formula 1, N-oxides, and salts thereof,

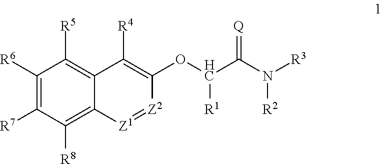

wherein
Q is O or S;
Z¹ and Z² are each independently CR⁹ or N; and
R¹, R², R³, R⁴, R⁵, R⁶, R⁷, R⁸ and R⁹ are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain bicyclic aryloxy carboxamides, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

World Patent Publication WO 2008/110355 discloses certain bicyclic aryloxy carboxamides as fungicides.

### SUMMARY OF THE INVENTION

This invention is directed to compounds of Formula 1 (including all stereoisomers), N-oxides, and salts thereof, agricultural compositions containing them and their use as fungicides:

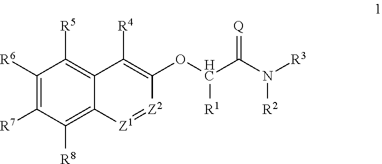

wherein
Q is O or S;
Z¹ and Z² are each independently CR⁹ or N;
R¹ is C₁-C₂ alkyl, C₂-C₄ alkenyl, C₂-C₄ alkynyl, C₁-C₄ haloalkyl, C₂-C₄ haloalkenyl, C₂-C₄ haloalkynyl, C₃-C₄ halocycloalkyl, C₄-C₅ cycloalkylalkyl, C₂-C₄ alkoxyalkyl, C₂-C₄ alkylthioalkyl, C₂-C₄ alkylsulfinylalkyl, C₂-C₄ alkylsulfonylalkyl, C₂-C₄ cyanoalkyl, C₂-C₆ alkylcarbonyl, C₂-C₆ alkoxycarbonyl, C₁-C₄ alkoxy or C₁-C₄ haloalkoxy;
R² is hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₂-C₆ cyanoalkyl, C₂-C₆ alkoxyalkyl, C₃-C₈ alkoxyalkoxyalkyl or benzyloxy(C₂-C₃ alkyl);
R³ is C₁-C₈ alkyl, C₂-C₈ alkenyl or C₂-C₈ alkynyl, each optionally substituted with substituents independently selected from halogen, hydroxy, cyano, nitro, amino, C(═O)OH, C(═O)NH₂, C(═O)R¹⁰, C(═O)OR¹¹, C(═O)NR¹²R¹³, OC(═O)R¹⁰, SC(═O)R¹⁰, OC(═O)OR¹¹, OC(═O)NR¹²R¹³, N(R¹²)C(═O)R¹⁰, N(R¹²)C(═O)OR¹¹, N(R¹²)C(═O)NR¹²R¹³, OSO₂R¹⁴, OSO₂NR¹²R¹³, NR¹²SO₂R¹⁴, NR¹²SO₂NR¹²R¹³, OR¹⁵, NR¹²R¹³, S(O)ₙR¹⁴, SO₂NR¹²R¹³, P(═O)(R¹⁷)₂, OP(═O)(R¹⁷)₂, Si(R¹⁸)₃, C(═NNR¹²R¹³)R¹⁹, N═CR¹⁹NR¹²R¹³, CH═NR²¹ and —CH[—O(CH₂)—]; or
R³ is NR¹²R¹³; or
R³ is a 3-, 4-, 5- or 6-membered saturated carbocyclic ring optionally substituted with up to 5 substituents independently selected from R²⁰; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR¹⁶)_{q}, the heterocyclic ring optionally substituted with up to 5 substituents independently selected from R²⁰ on carbon atom ring members and R^{20a} on nitrogen atom ring members;
R⁴, R⁵, R⁷ and R⁸ are each independently selected from hydrogen, halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₁-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino and C₂-C₆ alkylcarbonylamino;
R⁶ is halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₆ haloalkylamino, C₂-C₆ halodialkylamino or C₂-C₆ alkylcarbonylamino;
each R⁹ is independently selected from hydrogen, halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino and C₂-C₆ alkylcarbonylamino;
each R¹⁰ is independently selected from hydrogen, C₁-C₆ alkyl, C₁-C₆ haloalkyl, C₃-C₆ cycloalkyl and C₃-C₆ halocycloalkyl;
each R¹¹ is independently selected from C₁-C₆ alkyl, C₁-C₆ haloalkyl, C₃-C₆ cycloalkyl and C₃-C₆ halocycloalkyl;
each R¹² is independently selected from hydrogen, CHO, C₁-C₆ alkyl, C₁-C₆ haloalkyl and C₂-C₆ alkylcarbonyl;
each R¹³ is independently selected from CHO, C₁-C₆ alkyl, C₁-C₆ haloalkyl and C₂-C₆ alkylcarbonyl;
each R¹⁴ is independently C₁-C₆ alkyl or C₁-C₆ haloalkyl;
each R¹⁵ is independently selected from CHO, C₁-C₆ alkyl, C₁-C₆ haloalkyl and C₂-C₆ alkoxyalkyl;
each R¹⁶ and R¹⁹ is independently hydrogen or C₁-C₃ alkyl;
each R¹⁷ is independently C₁-C₆ alkyl or C₁-C₆ alkoxy;
each R¹⁸ is independently C₁-C₆ alkyl;
each R²⁰ is independently selected from halogen, cyano, C₁-C₆ alkyl, C₁-C₆ haloalkyl, C₂-C₆ alkylcarbonyl, C₁-C₆ alkoxy, C₁-C₆ haloalkoxy and C₁-C₆ alkylthio;
each R^{20a} is independently selected from cyano, C₁-C₆ alkyl, C₁-C₆ haloalkyl and C₂-C₆ alkylcarbonyl;
each R²¹ is a 5-membered unsaturated heterocyclic ring containing 2-4 carbon atoms and 1-3 nitrogen atoms as ring members, wherein the heterocyclic ring is optionally substituted with up to 2 substituents independently selected from R²⁰ on carbon atom ring members and R^{20a} on nitrogen atom ring members;
each n is independently 0, 1 or 2; and
p and q are independently 0, 1 or 2 in each instance of S(═O)ₚ(═NR¹⁶)_{q}, provided that the sum of p and q is 0, 1 or 2;
provided that
the compound of Formula 1 is other than 2-[(7-methoxy-2-naphthalenyl)oxy]-N-(2-propen-1-yl)-propanamide.

More particularly, this invention pertains to a compound of Formula 1 (including all stereoisomers), an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” also includes moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Cyanoalkyl” denotes an alkyl group substituted with one cyano group. Examples of “cyanoalkyl” include NCCH₂, NCCH₂, NCCH₂CH₂ and CH₃CH(CN)CH₂.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than one double bond such as 1,3- and 1,4-cyclohexadienyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. Examples of “haloalkoxyalkoxy” include CF₃OCH₂O—, ClCH₂CH₂OCH₂CH₂O—, Cl₃CCH₂OCH₂O— as well as branched alkyl derivatives.

“Alkylcarbonyl” denotes a straight-chain or branched alkyl moieties bonded to a C(═O) moiety. Examples of “alkylcarbonyl” include CH₃C(O), CH₃CH₂CH₂C(O) and (CH₃)₂CHC(O). Examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Examples of “alkylaminocarbonyl” include CH₃NHC(═O)—, CH₃CH₂NHC(═O)—, CH₃CH₂CH₂NHC(═O)—, (CH₃)₂CHNHC(═O)— and the different butylamino- or pentylaminocarbonyl isomers. Examples of “dialkylaminocarbonyl” include (CH₃)₂NC(═O)—, (CH₃CH₂)₂NC(═O)—, CH₃CH₂(CH₃)NC(═O)—, (CH₃)CHN(CH₃)C(═O)— and CH₃CH₂CH₂(CH₃)NC(═O)—.

“Alkylamino”, “dialkylamino” and the like, are defined analogously to the above examples. The term “halodialkylamino” denotes a dialkylamino group substituted on at least one alkyl moiety with one or more halogenatoms which may be the same or different. Examples of “halodialkylamino” include CF₃(CH₃)N—, (CF₃)₂N— and CH₂Cl(CH₃)N—.

“Trialkylsilyl” includes 3 branched and/or straight-chain alkyl radicals attached to and linked through a silicon atom, such as trimethylsilyl, triethylsilyl and tert-butyldimethylsilyl.

“—CHO(CH₂)” means

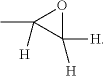

The total number of carbon atoms in a substituent group is indicated by the “Cᵢ-Cⱼ” prefix where i and j are numbers from 1 to 9. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CHCH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

Unless otherwise indicated, a “ring” as a component of Formula 1 (e.g., substituent R³) is carbocyclic or heterocyclic. The term “ring system” denotes two or more fused rings. The terms “bicyclic ring system” and “fused bicyclic ring system” denote a ring system consisting of two fused rings, in which either ring can be saturated, partially unsaturated, or fully unsaturated unless otherwise indicated. The term “fused heterobicyclic ring system” denotes a fused bicyclic ring system in which at least one ring atom is not carbon. The term “ring member” refers to an atom or other moiety (e.g., C(═O), C(═S), S(O) or S(O)₂) forming the backbone of a ring or ring system.

The terms “carbocyclic ring”, “carbocycle” or “carbocyclic ring system” denote a ring or ring system wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule. The term “aromatic ring system” denotes a carbocyclic or heterocyclic ring system in which at least one ring of the ring system is aromatic.

As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

When R³ is a 3-, 4-, 5- or 6-membered saturated carbocyclic ring, a cyclopropyl, cyclobutyl, cyclopentyl or cyclohexyl ring is attached to Formula 1 as defined in the Summary of the Invention and the ring may be optionally substituted with up to 5 substituents selected from a group of substituents as defined in the Summary of Invention.

When R³ is a 3-, 4-, 5- or 6-membered heterocyclic ring, the ring may be saturated or unsaturated and optionally substituted with up to 5 substituents selected from a group of substituents as defined in the Summary of Invention. When R³ is a 3-, 4-, 5- or 6-membered nitrogen-containing heterocyclic ring, it may be attached to the remainder of Formula 1 though any available carbon or nitrogen ring atom, unless otherwise described.

Also, as noted above, R³ can independently be (among others) a 3-, 4-, 5- or 6-membered heterocyclic ring, which may be saturated, partially unsaturated, or fully unsaturated and optionally substituted with up to 5 substituents selected from a group of substituents as defined in the Summary of Invention for R³. Optionally up to 3 carbon atom ring members of the heterocyclic ring are independently selected from C(═O), C(═S) and S(═O)ₚ(═NR⁸)_{q}. The definition of S(═O)ₚ(═NR⁸)_{q} includes the possibility of unoxidized sulfur atoms as ring members, because p and q can both be zero.

Examples of a 3-, 4-, 5- or 6-membered fully unsaturated heterocyclic ring include the rings U-1 through U-66 illustrated in Exhibit 1 wherein R^{v} is any substituent as defined in the Summary of the invention for R³ (i.e. R²⁰ on carbon ring members and R^{20a} on nitrogen ring members) and r is an integer from 0 to 5, limited by the number of available positions on each U-ring. As U-34, U-35, U-41, U-42, U-43, U-44, U-45, U-46, U-47 and U-48 have only one available position, for these U-rings r is limited to the integers 0 or 1, and r being 0 means that the U-ring is unsubstituted and a hydrogen is present at the position indicated by (R^{v})ᵣ.

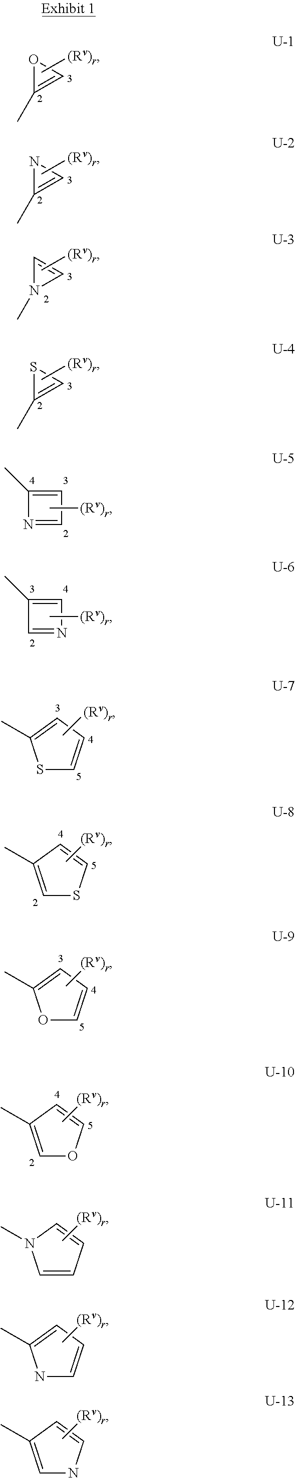

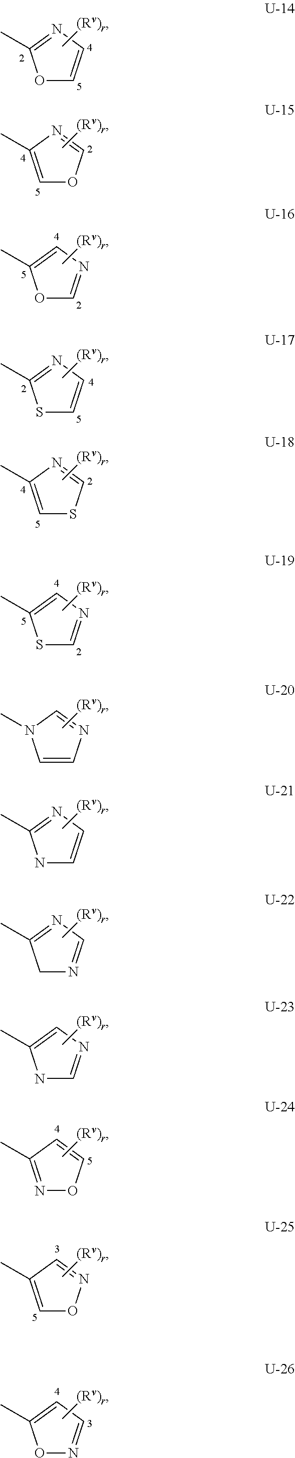

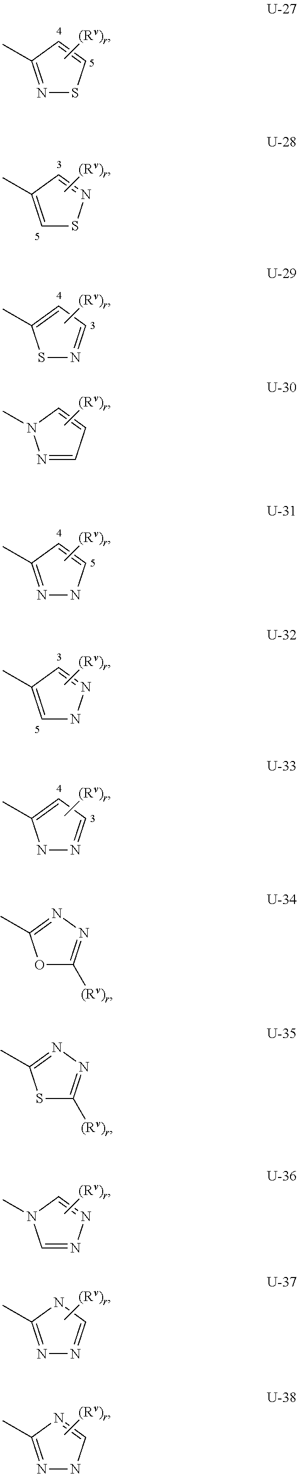

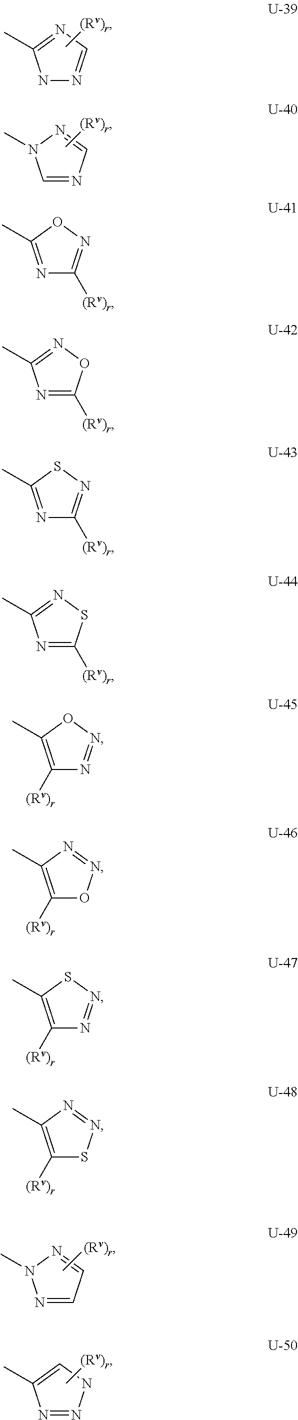

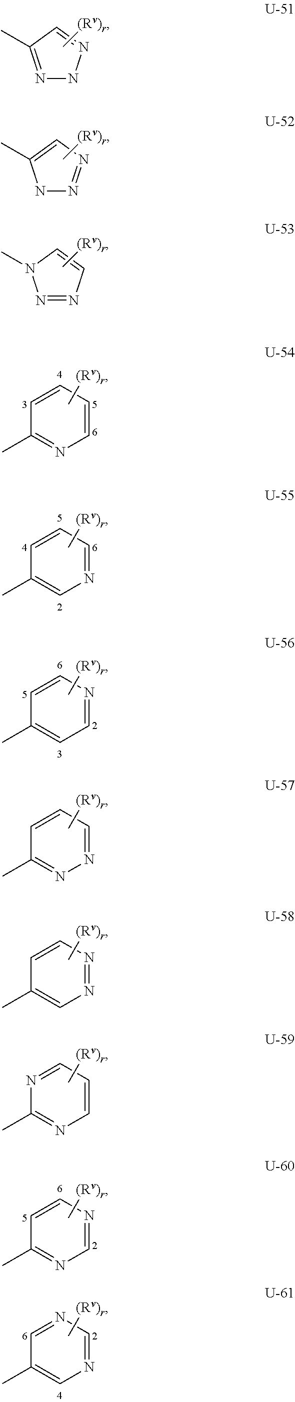

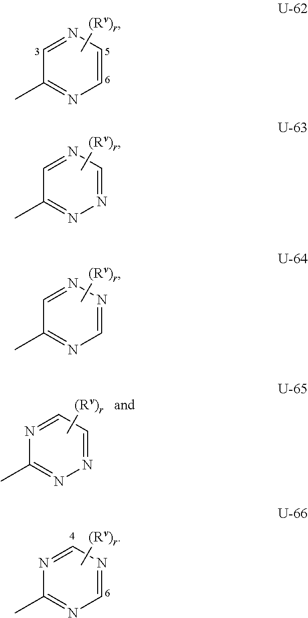

Although Rᵥ groups are shown on rings U-1 through U-66, it is noted that they do not need to be present since they are optional substituents. Note that when r is 0, this means the ring is unsubstituted. The nitrogen atoms that require substitution to fill their valence are substituted with H or R^{v}. Note that when the attachment point between (R^{v})ᵣ and the U-ring is illustrated as floating, (R^{V})ᵣ can be attached to any available carbon atom or nitrogen atom of the U-ring.

Examples of a 3-, 4-, 5- or 6-membered saturated or partially unsaturated heterocyclic ring include the rings G-1 through G-45 as illustrated in Exhibit 2 wherein R^{v} is any substituent as defined in the Summary of the Invention R³ (i.e. R²⁰ on carbon ring members and R^{20a} on nitrogen ring members) and r is an integer from 0 to 5, limited by the number of available positions on each G-ring. The optional substituents corresponding to (R^{V})ᵣ, can be attached to any available carbon or nitrogen by replacing a hydrogen atom. Note that when the attachment point on the G-ring is illustrated as floating, the G-ring can be attached to the remainder of Formula 1 through any available carbon or nitrogen of the G-ring by replacement of a hydrogen atom.

Note that when R³ comprises a ring selected from G-33, G-34, G-35 and G-41 through G-45, G² is O, S or N. Note that when G² is N, the nitrogen atom can complete its valence by substitution with either H or the substituents corresponding to R^{v} as defined in the Summary of Invention for R³.

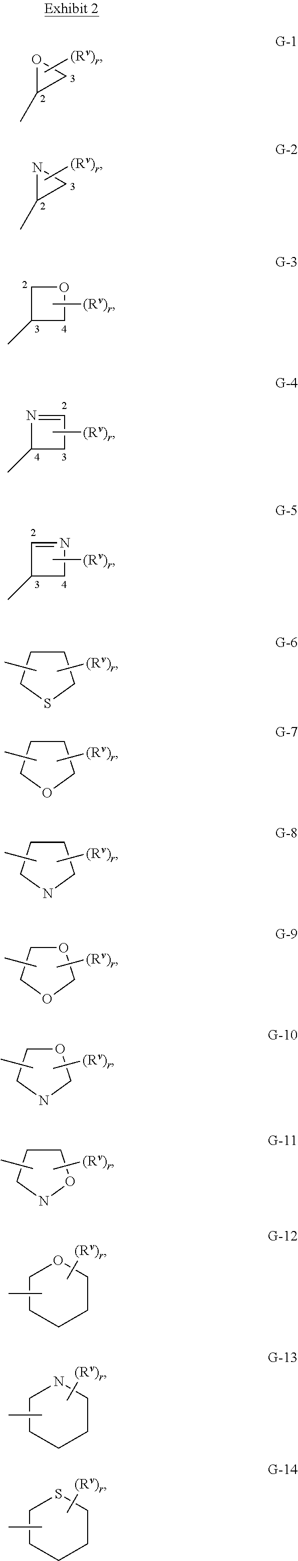

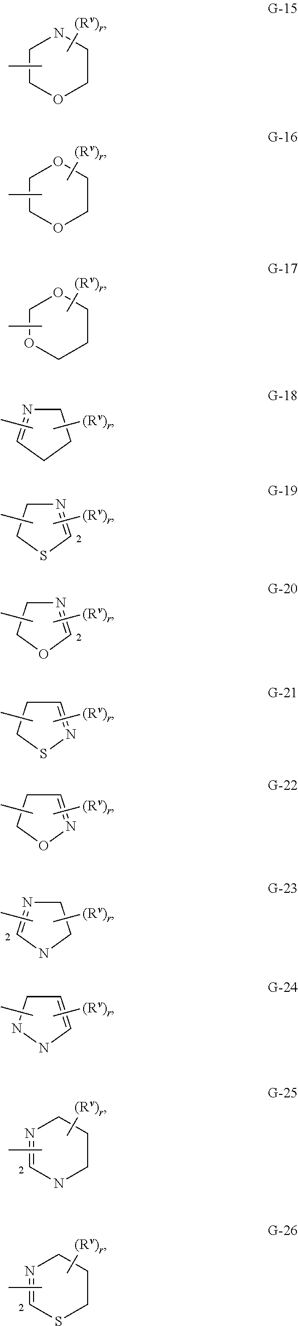

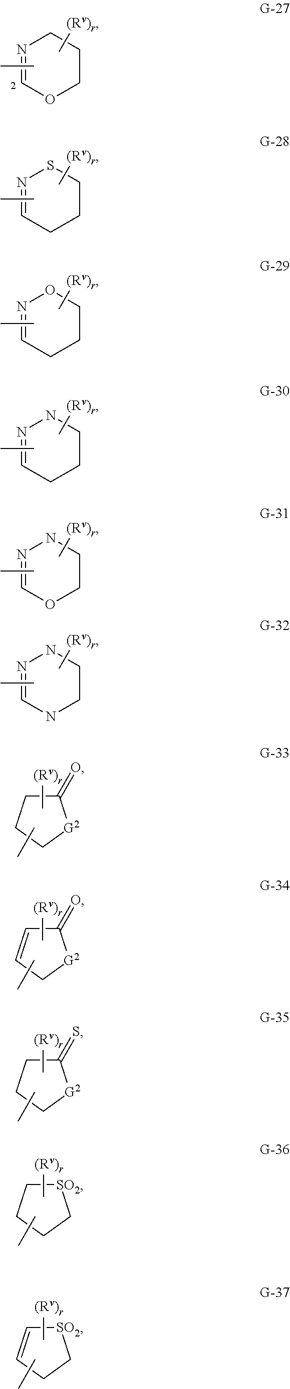

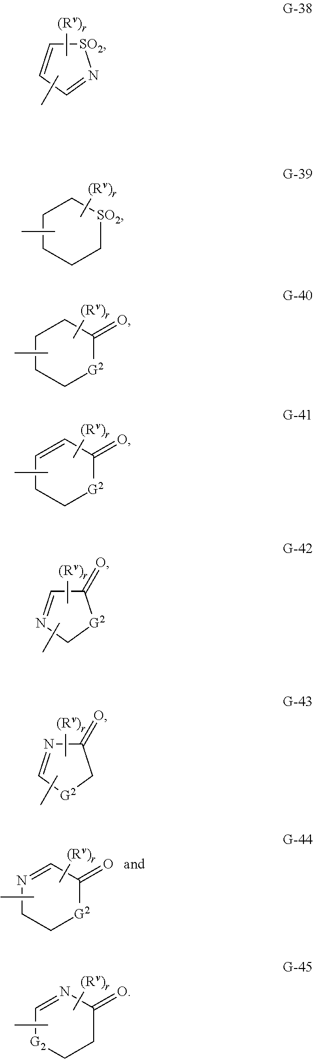

R²¹ is a 5-membered unsaturated heterocyclic ring. Examples of a 5-membered unsaturated heterocyclic ring containing 2-4 carbon atoms and 1-3 nitrogen atoms are in Exhibit 1 (U-11, U-12, U-13, U-20, U-21, U-22, U-23, U-30, U-31, U-32, U-33, U-36, U-37, U-38, U-39, U-40, U-49, U-50, U-51, U-52 and U-53) and Exhibit 2 (G-18, G-23 and G-24). The ring may be optionally substituted with up to 2 substituents independently selected from R²⁰ on carbon atoms and R^{20a} on nitrogen atom ring members.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include (where Formula 1 as used in the following Embodiments includes N-oxides and salts thereof):
Embodiment 1. A compound of Formula 1 wherein Q is O.
Embodiment 2. A compound of Formula 1 or Embodiment 1 wherein Z² is CR⁹.
Embodiment 3. A compound of Formula 1 or Embodiment 1 or 2 wherein Z¹ is CR⁹.
Embodiment 4. A compound of Formula 1 or any one of Embodiments 1 through 3 wherein R¹ is C₁-C₂ alkyl or C₁-C₄ alkoxy.
Embodiment 5. A compound of Embodiment 4 wherein R¹ is methyl, ethyl or methoxy.
Embodiment 5a. A compound of Embodiment 5 wherein R¹ is methyl or ethyl.
Embodiment 5b. A compound of Embodiment 5 wherein R¹ is methoxy.
Embodiment 6. A compound of Formula 1 or any one of Embodiments 1 through 5 wherein R² is hydrogen.
Embodiment 7a. A compound of Formula 1 or any one of Embodiments 1 through 6 wherein R³ is C₁-C₈ alkyl or C₃-C₈ alkynyl, each optionally substituted with up to 3 substituents independently selected from cyano, hydroxy, fluoro, OR¹⁵ and CHO.
Embodiment 7. A compound of Formula 1 or any one of Embodiments 1 through 6 wherein R³ is C₁-C₈ alkyl or C₃-C₈ alkynyl, each optionally substituted with up to 3 substituents independently selected from hydroxy, fluoro, OR¹⁵ and CHO.
Embodiment 7b. A compound of Formula 1 or any one of Embodiments 1 through 6 wherein R³ is C₁-C₈ alkyl or C₃-C₈ alkynyl, each optionally substituted with up to 1 substituent independently selected from cyano or OR¹⁵.
Embodiment 8a. A compound of Embodiment 7a wherein R³ is C₁-C₈ alkyl optionally substituted with cyano or OR¹⁵.
Embodiment 8b. A compound of Embodiment 7a wherein R³ is C₃-C₈ alkynyl.
Embodiment 8. A compound of Embodiment 7 wherein R³ is C₁-C₈ alkyl optionally substituted with OR¹⁵ or fluoro.
Embodiment 9a. A compound of Embodiment 8a wherein R³ is C(CH₃)₂CN, C(CH₃)₂CH₂OCH₃, C(CH₃)₂CH₂OCH₂OCH₃ or C(CH₃)₃.
Embodiment 9. A compound of Embodiment 8 wherein R³ is C(CH₃)₂CH₂OCH₃, C(CH₃)₂CH₂OCH₂OCH₃, C(CH₃)₃ or C(CH₃)₂(CH₂F).
Embodiment 9b. A compound of Embodiment 8 wherein R³ is C(CH₃)₂CH₂OCH₃, C(CH₃)₂CH₂OCH₂OCH₃ or C(CH₃)₃.
Embodiment 9c. A compound of Embodiment 9 wherein R³ is C(CH₃)₂CH₂OCH₃.
Embodiment 9d. A compound of Embodiment 9 wherein R³ is C(CH₃)₃.
Embodiment 9e. A compound of Embodiment 8b wherein R³ is C(CH₃)₂C≡CH or C(CH₃)₂C≡CCH₃.
Embodiment 10. A compound of Formula 1 or any one of Embodiments 1 through 9 wherein R³ includes a tertiary carbon atom, and R³ is bonded to the remainder of Formula 1 through the tertiary carbon atom.
Embodiment 11. A compound of Formula 1 or any one of Embodiments 1 through 10 wherein R⁴, R⁵, R⁷, R⁸ and R⁹ are each independently selected from hydrogen and C₁-C₆ alkyl.
Embodiment 12. A compound of Embodiment 11 wherein R⁴, R⁵, R⁷, R⁸ and R⁹ are each independently selected from hydrogen, methyl and ethyl.
Embodiment 13a. A compound of Formula 1 or any one of Embodiments 1 through 12 wherein R⁶ is halogen, C₁-C₂ alkoxy, C₁-C₂ haloalkoxy or C₂-C₃ alkynyl.
Embodiment 13. A compound of Formula 1 or any one of Embodiments 1 through 12 wherein R⁶ is halogen, C₁-C₂ alkoxy, C₁-C₂ haloalkoxy or C₂ alkynyl.
Embodiment 14. A compound of Embodiment 13 wherein R⁶ is halogen, C₁-C₂ alkoxy or C₁-C₂ haloalkoxy.
Embodiment 14a. A compound of Embodiment 13a wherein R⁶ is halogen, C₁-C₂ alkoxy or C₂-C₃ alkynyl.
Embodiment 15. A compound of Embodiment 14 wherein R⁶ is bromo, iodo, methoxy or difluoromethoxy.
Embodiment 15a. A compound of Embodiment 14a wherein R⁶ is bromo, methoxy, ethoxy, C≡CH or C≡CCH₃.
Embodiment 16. A compound of Formula 1 or any one of Embodiments 1 through 15 wherein R¹⁰ is hydrogen or C₁-C₆ alkyl.
Embodiment 17. A compound of Embodiment 16 wherein R¹⁰ is hydrogen.
Embodiment 18. A compound of Formula 1 or any one of Embodiments 1 through 17 wherein R¹² is hydrogen or C₁-C₆ alkyl.
Embodiment 19. A compound of Formula 1 or any one of Embodiments 1 through 18 wherein R¹³ is CHO or C₁-C₆ alkyl.
Embodiment 20. A compound of Formula 1 or any one of Embodiments 1 through 19 wherein R¹⁵ is CHO, C₁-C₆ alkyl or C₂-C₆ alkoxyalkyl.
Embodiment 21. A compound of Embodiment 20 wherein R¹⁵ is CHO, methyl or methoxymethyl.
Embodiment 21a. A compound of Embodiment 20 wherein R¹⁵ is methyl or methoxymethyl.

Embodiments of this invention, including Embodiments 1-21 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-21 above as well as any other embodiments described herein, and any combination thereof, pertain to the compositions and methods of the present invention.

Combinations of Embodiments 1-22 are illustrated by:

Embodiment AA. A compound of Formula 1 wherein
Q is O or S;
Z¹ and Z² are each independently CR⁹ or N;
R¹ is C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₃-C₄ cycloalkyl, C₃-C₄ halocycloalkyl, C₄-C₅ cycloalkylalkyl, C₂-C₄ alkoxyalkyl, C₂-C₄ alkylthioalkyl, C₂-C₄ alkylsulfinylalkyl, C₂-C₄ alkylsulfonylalkyl, C₂-C₄ cyanoalkyl, C₂-C₆ alkylcarbonyl, C₂-C₆ alkoxycarbonyl, C₁-C₄ alkoxy or C₁-C₄ haloalkoxy;
R² is hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₂-C₆ cyanoalkyl, C₂-C₆ alkoxyalkyl, C₃-C₈ alkoxyalkoxyalkyl or benzyloxy(C₂-C₃ alkyl);
R³ is C₁-C₈ alkyl, C₂-C₈ alkenyl or C₂-C₈ alkynyl, each optionally substituted with substituents independently selected from halogen, hydroxy, cyano, nitro, amino, C(═O)OH, C(═O)NH₂, C(═O)R¹⁰, C(═O)OR¹¹, C(═O)NR¹²R¹³, OC(═O)R¹⁰, SC(═O)R¹⁰, OC(═O)OR¹¹, OC(═O)NR¹²R¹³, N(R¹²)C(═O)R¹⁰, N(R¹²)C(═O)OR¹¹, N(R¹²)C(═O)NR¹²R¹³, OSO₂R¹⁴, OSO₂NR¹²R¹³, NR¹²SO₂R¹⁴, NR¹²SO₂NR¹²R¹³, OR¹⁵, NR¹²R¹³, S(O)ₙR¹⁴, SO₂NR¹²R¹³, P(═O)(R¹⁷)₂, OP(═O)(R¹⁷)₂, Si(R¹⁸)₃, C(═NNR¹²R¹³)R¹⁹, N═CR¹⁹NR¹²R¹³, CH═NR²¹ and —CHO(CH₂); or
R³ is NR¹²R¹³; or
R³ is a 3-, 4-, 5- or 6-membered saturated carbocyclic ring optionally substituted with up to 5 substituents independently selected from R²⁰; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR¹⁶)_{q}, the heterocyclic ring optionally substituted with up to 5 substituents independently selected from R²⁰ on carbon atom ring members and R^{20a} on nitrogen atom ring members;
R⁴, R⁵, R⁷ and R⁸ are each independently selected from hydrogen, halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino and C₂-C₆ alkylcarbonylamino;
R⁶ is halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₆ haloalkylamino, C₂-C₆ halodialkylamino or C₂-C₆ alkylcarbonylamino;
each R⁹ is independently selected from hydrogen, halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino and C₂-C₆ alkylcarbonylamino;
each R¹⁰ is independently selected from hydrogen, C₁-C₆ alkyl, C₁-C₆ haloalkyl, C₃-C₆ cycloalkyl and C₃-C₆ halocycloalkyl;
each R¹¹ is independently selected from C₁-C₆ alkyl, C₁-C₆ haloalkyl, C₃-C₆ cycloalkyl and C₃-C₆ halocycloalkyl;
each R¹² is independently selected from hydrogen, CHO, C₁-C₆ alkyl, C₁-C₆ haloalkyl and C₂-C₆ alkylcarbonyl;
each R¹³ is independently selected from CHO, C₁-C₆ alkyl, C₁-C₆ haloalkyl and C₂-C₆ alkylcarbonyl;
each R¹⁴ is independently C₁-C₆ alkyl or C₁-C₆ haloalkyl;
each R¹⁵ is independently selected from CHO, C₁-C₆ alkyl, C₁-C₆ haloalkyl and C₂-C₆ alkoxyalkyl;
each R¹⁶ and R¹⁹ is independently hydrogen or C₁-C₃ alkyl;
each R¹⁷ is independently C₁-C₆ alkyl or C₁-C₆ alkoxy;
each R¹⁸ is independently C₁-C₆ alkyl;
each R²⁰ is independently selected from halogen, cyano, C₁-C₆ alkyl, C₁-C₆ haloalkyl, C₂-C₆ alkylcarbonyl, C₁-C₆ alkoxy, C₁-C₆ haloalkoxy and C₁-C₆ alkylthio;
each R^{20a} is independently selected from cyano, C₁-C₆ alkyl, C₁-C₆ haloalkyl and C₂-C₆ alkylcarbonyl;
each R²¹ is a 5-membered unsaturated heterocyclic ring containing 2-4 carbon atoms and 1-3 nitrogen atoms as ring members, wherein the heterocyclic ring is optionally substituted with up to 2 substituents independently selected from R²⁰ on carbon atom ring members and R^{20a} on nitrogen atom ring members;
each n is independently 0, 1 or 2; and
p and q are independently 0, 1 or 2 in each instance of S(═O)ₚ(═NR¹⁶)_{q}, provided that the sum of p and q is 0, 1 or 2;
provided that
the compound of Formula 1 is other than 2-[(7-methoxy-2-naphthalenyl)oxy]-N-(2-propen-1-yl)-propanamide.

Embodiment A. A compound of Formula 1 as described in the Summary of the Invention or Embodiment AA wherein
Q is O;
Z² is CR⁹;
R¹ is C₁-C₄ alkyl or C₁-C₄ alkoxy;
R² is hydrogen;
R³ is C₁-C₈ alkyl or C₂-C₈ alkynyl, each optionally substituted with up to 3 substituents independently selected from hydroxy, OR¹⁵ and CHO;
R⁴, R⁵, R⁷, R⁸ and R⁹ are each independently selected from hydrogen and C₁-C₆ alkyl; and
R⁶ is halogen, C₁-C₂ alkoxy, C₁-C₂ haloalkoxy or C₂ alkynyl.

Embodiment A1. A compound of Formula 1 as described in the Summary of the Invention or Embodiment AA wherein
Q is O;
Z² is CR⁹;
R¹ is C₁-C₂ alkyl or C₁-C₄ alkoxy;
R² is hydrogen;
R³ is C₁-C₈ alkyl or C₂-C₈ alkynyl, each optionally substituted with up to 3 substituents independently selected from cyano, hydroxy, OR¹⁵ and CHO;
R⁴, R⁵, R⁷, R⁸ and R⁹ are each independently selected from hydrogen and C₁-C₆ alkyl; and
R⁶ is halogen, C₁-C₂ alkoxy, C₁-C₂ haloalkoxy or C₂-C₃ alkynyl.

Embodiment B. A compound of Embodiment A wherein
Z¹ is CR⁹;
R¹ is methyl, ethyl or methoxy;
R³ is C₁-C₈ alkyl optionally substituted with OR¹⁵ or fluoro;
R⁴, R⁵, R⁷, R⁸ and R⁹ are each independently selected from hydrogen, methyl and ethyl; and
R⁶ is halogen, C₁-C₂ alkoxy or C₁-C₂ haloalkoxy.

Embodiment B1. A compound of Embodiment A1 wherein
Z¹ is CR⁹;
R¹ is methyl, ethyl or methoxy;
R³ is C₁-C₈ alkyl optionally substituted with cyano or OR¹⁵;
R⁴, R⁵, R⁷, R⁸ and R⁹ are each independently selected from hydrogen, methyl and ethyl; and
R⁶ is halogen, C₁-C₂ alkoxy or C₂-C₃ alkynyl.

Embodiment B2. A compound of Embodiment A1 wherein
Z¹ is CR⁹;
R¹ is methyl, ethyl or methoxy;
R³ is C₃-C₈ alkynyl;
R⁴, R⁵, R⁷, R⁸ and R⁹ are each independently selected from hydrogen, methyl and ethyl; and
R⁶ is halogen, C₁-C₂ alkoxy or C₂-C₃ alkynyl.

Embodiment C. A compound of Embodiment B wherein
R³ is C(CH₃)₂CH₂OCH₃, C(CH₃)₂CH₂OCH₂OCH₃, C(CH₃)₃ or C(CH₃)₂(CH₂F); and
R⁶ is bromo, iodo, methoxy or difluoromethoxy.

Embodiment C1. A compound of Embodiment B1 wherein
R³ is C(CH₃)₂CN, C(CH₃)₂CH₂OCH₃, C(CH₃)₂CH₂OCH₂OCH₃ or C(CH₃)₃; and
R⁶ is bromo, methoxy, ethoxy, C≡CH or C≡CCH₃.

Embodiment C2. A compound of Embodiment B2 wherein
R³ is C(CH₃)₂C≡CH or C(CH₃)₂C≡CCH₃; and
R⁶ is bromo, methoxy, ethoxy, C≡CH or C≡CCH₃.

Specific embodiments include compounds of Formula 1 selected from the group consisting of:
2-[(7-bromo-2-naphthalenyl)oxy]-N-(2-methoxy-1,1-dimethylethyl)butanamide;
2-[(7-bromo-2-naphthalenyl)oxy]-N-[2-(methoxymethoxy)-1,1-dimethylethyl]butanamide;
2-[(6-iodo-3-quinolinyl)oxy]-N-(2-methoxy-1,1-dimethylethyl)butanamide;
N-(1,1-dimethylethyl)-2-[(6-iodo-3-quinolinyl)oxy]butanamide;
N-(1,1-dimethylethyl)-2-[(7-methoxy-2-naphthalenyl)oxy]butan amide;
2-[(7-bromo-2-naphthalenyl)oxy]-N-(1,1-dimethylethyl)butanamide;
2-[(7-bromo-2-naphthalenyl)oxy]-N-[2-[[(dimethylamino)methylene]amino]-1,1-dimethylethyl]butanamide;
2-[(7-bromo-2-naphthalenyl)oxy]-N-(2-methoxy-1,1-dimethylethyl)propanamide and
2-[(7-bromo-2-naphthalenyl)oxy]-N-(2-fluoro-1,1-dimethylethyl)butanamide.

Further specific embodiments include compounds of Formula 1 selected from the group consisting of:
2-[(7-ethynyl-2-naphthalenyl)oxy]-N-(2-methoxy-1,1-dimethylethyl)butanamide;
2-[(7-ethynyl-2-naphthalenyl)oxy]-2-methoxy-N-(2-methoxy-1,1-dimethylethyl)acetamide;
2-[(7-ethynyl-2-naphthalenyl)oxy]-N-[2-(methoxymethoxy)-1,1-dimethylethyl]butanamide;
N-(1-cyano-1-methylethyl)-2-[(7-ethynyl-2-naphthalenyl)oxy]butanamide;
N-(1,1-dimethyl-2-butyn-1-yl)-2-[(7-ethynyl-2-naphthalenyl)oxy]butanamide;
N-(1,1-dimethylethyl)-2-[(7-ethynyl-2-naphthalenyl)oxy]butanamide;
2-[(7-bromo-2-naphthalenyl)oxy]-2-methoxy-N-(2-methoxy-1,1-dimethylethyl)acetamide;
N-(2-methoxy-1,1-dimethylethyl)-2-[[7-(1-propyn-1-yl)-2-naphthalenyl]oxy]butanamide;
2-methoxy-N-(2-methoxy-1,1-dimethylethyl)-2-[[7-(1-propyn-1-yl)-2-naphthalenyl]oxy]acetamide;
N-(1,1-dimethylethyl)-2-[(7-ethoxy-2-naphthalenyl)oxy]butanamide;
N-(1,1-dimethyl-2-propyn-1-yl)-2-[(7-ethoxy-2-naphthalenyl)oxy]butanamide; and
N-(1,1-dimethyl-2-propyn-1-yl)-2-[(7-ethoxy-2-naphthalenyl)oxy]-2-methoxyacetamide.

This invention provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as embodiment of such methods are methods comprising applying a fungicidally effective amount of a compound corresponding to any of the compound embodiments describe above. Of particular notes are embodiment where the compounds are applied as compositions of this invention.

One or more of the following methods and variations as described in Schemes 1-7 can be used to prepare the compounds of Formula 1. The definitions of R¹, R², R³, R⁴, R⁵, R⁶, R⁷, R⁸, Z¹, Z² and Q in the compounds of Formulae 1-8 below are as defined above in the Summary of the Invention unless otherwise noted. Formulae 1a-1b are various subsets of Formula 1, and all substituents for Formulae 1a-1b are as defined above for Formula 1 unless otherwise noted.

Compounds of Formula 1a (i.e. Formula 1 where Q is O) can be prepared by the reaction of 1 molar equivalent of a compound of Formula 2 with 1-1.5 molar equivalents of an activating reagent such as 2-chloro-1-methylpyridinium iodide and 1-5 molar equivalents of a compound of Formula 3 in the presence of 1-10 molar equivalents of a base such as N,N-diisopropylethylamine as shown in Scheme 1. The reaction can be carried out in an inert solvent such as diethyl ether, tetrahydrofuran, methylene chloride, toluene or xylene at temperatures, between −20 to 60° C., and preferably from −10° C. to ambient temperatures for a period of time ranging from 1 hour to 4 days. The reaction mixture is then concentrated and the residue is column chromatographed (over silica gel with eluents such as solutions of ethyl acetate or dichloromethane in hexanes) to give the desired compound of Formula 1a.

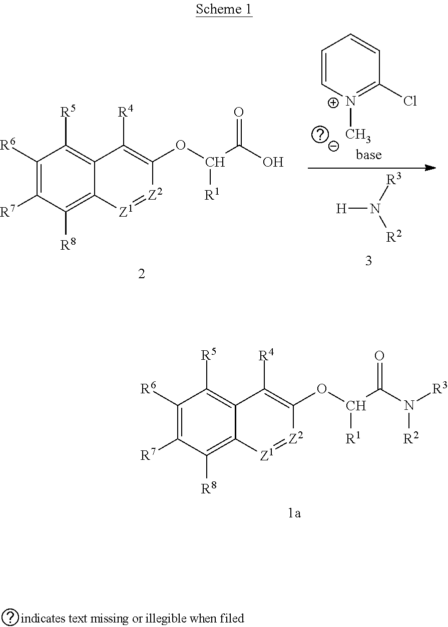

Alternatively, compounds of Formula 1a (i.e. Formula 1 wherein Q is O) can also be prepared by the reaction of compounds of Formula 4 with compounds of Formula 5 in the presence of a base such as Cs₂CO₃, K₂CO₃ or KOC(CH₃)₃ as shown in Scheme 2. The reaction can be carried out by adding 0.7 to 3 molar equivalent of a compound of Formula 5 to a suspension or solution prepared by adding 1-20 molar equivalents of a base such as Cs₂CO₃, K₂CO₃, or KOC(CH₃)₃ to a solution of 1 molar equivalent of a compound of Formula 4 in an solvent such as acetone, diethyl ether, tetrahydrofuran, methylene chloride, N,N-dimethylformamide, t-butanol or toluene under N₂ at temperatures between −20 to 30° C. After the addition, the reaction mixture can be stirred at temperatures between −20 to 110° C., preferably from 10° C. to 90° C., for a period of time ranging from 1 hour to 4 days. The reaction mixture is then brought to room temperature and filtered. The filtrate is dried over a drying agent such as MgSO₄ or Na₂SO₄ and then concentrated. The residue is column chromatographed (over silica gel with eluents such as solutions of ethyl acetate or dichloromethane in hexanes) to give the desired compounds of Formula 1a.

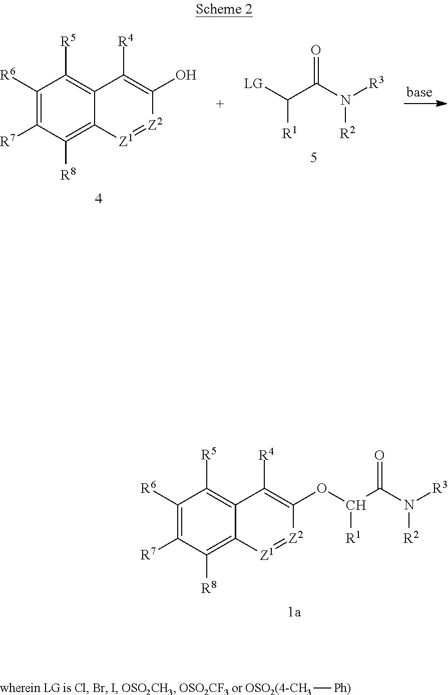

Compounds of Formula 1b (i.e. Formula 1 wherein Q is S) can be prepared by treating compounds of Formula 1a with Lawesson's reagent or P₂S₅ as shown in Scheme 3 using methods taught in: Heterocycles 1995, 40, 271-8; J. Med. Chem. 2008, 51, 8124-8134; J. Med. Chem. 1990, 33, 2697-706; Synthesis 1989, 396-7; J. Chem. Soc., Perkin Trans. 1, 1988, 1663-8; Tetrahedron 1988 44, 3025-36; J. Org. Chem. 1988 53, 1323-6 or slight modification thereof.

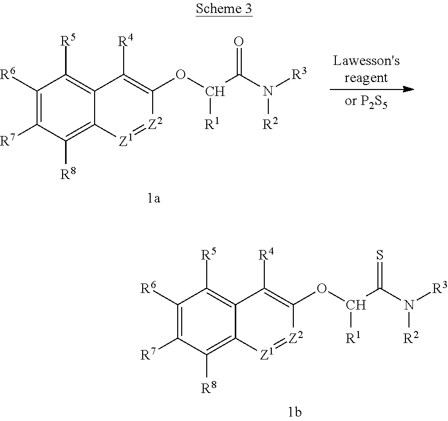

Compounds of Formula 2 can be prepared by the reaction of 1 molar equivalent of a compound of Formula 6 with 1-35 molar equivalents of a hydroxide base (e.g. NaOH, LiOH or KOH) as shown in Scheme 4. The reaction can be carried out in a solvent mixture containing water and an organic solvent such as tetrahydrofuran, or methanol at temperatures between 0 to 70° C., and preferably from 0 to 35° C. for a period of time ranging from 1 hour to 4 days. The reaction mixture is then acidified by addition of an acid such as hydrochloric acid and extracted with an organic solvent such as ethyl acetate. The organic layer is dried over a drying agent such as sodium sulfate or magnesium sulfate and is then concentrated to give the desired compound of Formula 2.

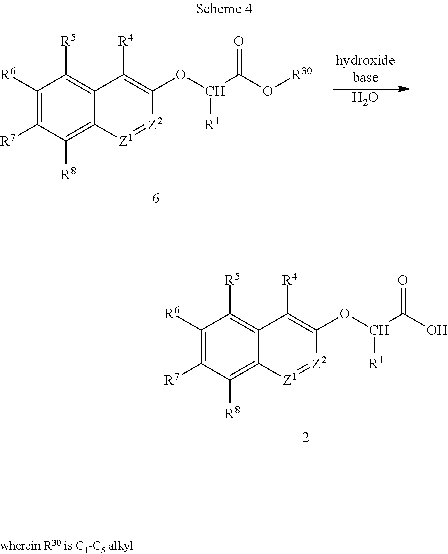

Compounds of Formula 6 can be prepared by the reaction of compounds of Formula 4 with compounds of Formula 7 in the presence of a base such as Cs₂CO₃, K₂CO₃ or KOC(CH₃)₃ as shown in Scheme 5. The reaction can be carried out by adding 0.7 to 3 molar equivalent of a compound of Formula 7 to a suspension or solution prepared by adding 1-20 molar equivalents of a base such as Cs₂CO₃, K₂CO₃ or KOC(CH₃)₃ to a solution of 1 molar equivalents of a compound of Formula 4 in an solvent such as acetone, diethyl ether, tetrahydrofuran, methylene chloride, N,N-dimethylformamide, t-butanol or toluene under N₂ at temperatures between −20 to 30° C. After the addition, the reaction mixture can be stirred at temperatures between −20 to 110° C., preferably from 10 to 90° C., for a period of time ranging from 1 hour to 4 days. The reaction mixture is then brought to room temperature and filtered. The filtrate is dried over a drying agent such as MgSO₄ or Na₂SO₄ and then concentrated. The residue is column chromatographed (over silica gel with eluents such as solutions of ethyl acetate or dichloromethane in hexanes) to give the desired compounds of Formula 6.

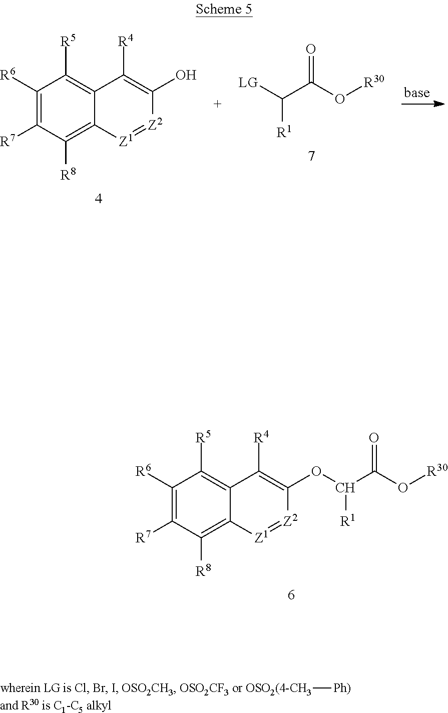

Compounds of Formula 3 are either commercially available or can be prepared by one skilled in the art using methods taught in the following references or slight modifications thereof: Tetrahedron 2009, 65, 638-643; J. Med. Chem. 2008, 51, 7380-7395; J. Am. Chem. Soc. 2008, 130, 8923-8930; Angew. Chem. Int. Ed. Eng. 2007, 46, 7259-7261; Synthesis 2006, 4143-4150; Biooig. Med. Chem. 2005, 13, 5463-5474; Tetrahedron 1994, 50, 5335-44; World Patent Publication WO 2006/058700; and World Patent Publication WO 2007/022900.

Compounds of Formula 4 are either commercially available or can be prepared by one skilled in the art using methods taught in the following references or slight modifications thereof: Chemistry Letters 1994, 597-600; Organic Letters 2007, 9, 5259-5262; J. Med. Chem. 2005, 48, 3953-3979; Tetrahedron 2004, 60, 4019-4029; J. Am. Chem. Soc. 2002, 124, 5380-5401; Synlett 1997, 1187-1189; Synthesis 1998, 729-734; J. Med. Chem. 1999, 42, 3557-3571; J. Med. Chem. 2007, 50, 6554-6569; World Patent Publication WO 2008/008539; World Patent Publication WO 2008/103277; and World Patent Publication WO 2005/082880.

Compounds of Formula 4a (i.e. Formula 4 wherein Z¹ is N, Z² is CH and R⁴ is H) can be prepared according to the method of Scheme 6 by the refluxing of compounds of Formula 4b (i.e. Formula 4 wherein Z¹ is N, Z² is CH and R⁴ is CO₂H) in high-boiling aromatic or nonaromatic solvents such as toluene, benzene or nitrobenzene, N-methyl-2-pyrrolidone or N,N-dimethylformamide in a temperature range between 50 to 300° C., and preferably from 180 to 250° C. The reaction is carried out by adding a compound of Formula 4b to a reaction vessel containing pre-heated solvent and stirring for a time period between 10 min and 6 h or until the evolution of carbon dioxide has ceased. The reaction product is isolated as a solid from filtering the hot reaction mixture and then further purified via crystallization from various mixtures of water and polar solvents such as alcohols or N,N-dimethylformamide to afford compounds of Formula 4a.

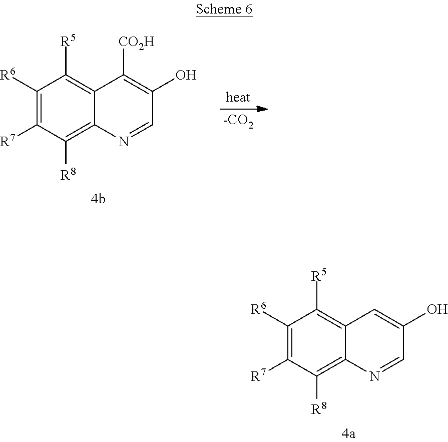

Compounds of Formula 5 are either commercially available or can be prepared by one skilled in the art using methods taught in the following references or slight modifications thereof: World Patent Publication WO 2006/113552; J. Am. Chem. Soc. 2006, 128, 4976-4985; J. Org. Chem. 2006, 71, 1471-1479; U.S. Patent Publication US 2005/143381; J. Chem. Res., Synop. 1995, 166-7; Tetrahedron, Asymmetry 1993, 4, 1105-12; World Patent Publication WO 2008/110355; Tetrahedron 2008, 64, 3197-3203; Organic Letters 2006, 8, 2843-2846; and J. Med. Chem. 2003, 46, 691-701.

Compounds of Formula 7 are either commercially available or can be prepared by one skilled in the art using methods taught in the following references or slight modifications thereof: World Patent Publication WO 2007/136571; Tetrahedron 2008, 64, 8155-8158; J. Org. Chem. 2008, 73, 4721-4724; J. Fluor. Chem. 2007, 128, 1271-1279; European Patent Publication EP 1806339; Angew. Chem. Int. Ed. Eng. 2008, 47, 7511-7514; Tetrahedron 2008, 64, 5085-5090; and Bioorg. Med. Chem. 2007, 15, 2827-2836.

Compounds of Formula 4b (i.e. Formula 4 wherein Z¹ is N, Z² is CH and R⁴ is CO₂H) can be prepared by the reaction of compounds of Formula 8 with bromo-pyruvic acid or ethyl bromopyruvate in the presence of bases such as KOH, NaOH or LiOH in aqueous reaction mixtures as shown in Scheme 7. The reaction is carried out by adding the compound of Formula 8 to a solution of 5 to 20 molar equivalents of the hydroxide base in water which is warmed by dissolution of the base. The reaction mixture is stirred until it reaches ambient conditions and is then treated with 1 to 20 molar equivalents of pyruvic acid in one portion with stirring at 25° C. for a period of 2 to 30 days. The compound of Formula 8 is precipitated from the reaction mixture by acidification to pH 1 to 5 with concentrated acids such as HCl or HBr. The solids are collected and washed with various mixtures of ethanol and water and air dried to afford compounds of Formula 4b.

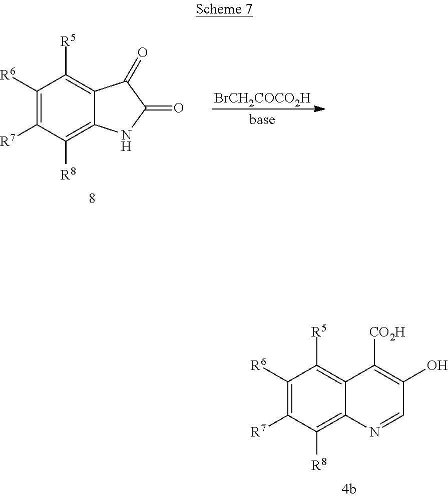

Compounds of Formula 8 are either commercially available or can be prepared by one skilled in the art using methods taught in the following references or slight modifications thereof: Synlett 2008, 2023-2027; J. Med. Chem. 2007, 50, 21-39; J. Org. Chem. 2006, 71, 5921-5929; Synthesis 2003, 2047-2052.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described, in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, “br s” means broad singlet.

### Example 1

### Preparation of 2-[(7-bromo-2-naphthalenyl)oxy]-N-(2-methoxy-1,1-dimethylethyl)butanamide (Compound 7)

### Step A: Preparation of methyl 2-[(7-bromo-2-naphthalenyl)oxy]butanoate

To a solution of 7-bromo-2-naphthol (4.5 g, 20 mmol) in acetone (200 mL) was added cesium carbonate (16.4 g, 50 mmol) at room temperature under nitrogen atmosphere with stirring. After the addition, the mixture was stirred at the room temperature for 3 minutes and then methyl-2-bromobutyrate (5.4 g, 30 mmol) was added. The mixture was stirred under nitrogen atmosphere at reflux overnight. The reaction mixture was then cooled to room temperature and filtered. The filtrate was concentrated under reduced pressure and the resultant residue was purified by column chromatography (ethyl acetate in hexanes in volume ratios from 10 to 40% as eluents) to give the title compound (6.08 g) as a solid.

¹H NMR (CDCl₃) δ 1.11 (t, 3H), 2.05 (m, 2H), 3.75 (s, 3H), 4.71 (t, 1H), 6.95 (s, 1H), 7.2 (d, 1H), 7.39 (d, 1H), 7.59 (d, 1H), 7.7 (d, 1H), 7.84 (s, 1H).

### Step B: Preparation of 2-[(7-bromo-2-naphthalenyl)oxy]butanoic acid

To a solution of methyl 2-[(7-bromo-2-naphthalenyl)oxy]butanoate (i.e. the product of Step A) (6.08 g, 18.8 mmol) in tetrahydrofuran (27 mL) was added aqueous sodium hydroxide solution (35 mL of 1 N, 35 mmol) at room temperature with stirring. The mixture was stirred at room temperature for 3 hours, acidified with 37% hydrochloric acid (3.6 mL) and then extracted with ethyl acetate (3×60 mL). The organic phases were combined, washed with brine (60 mL), dried (MgSO₄) and concentrated under reduced pressure to give the title compound (4.45 g) as a solid.

¹H NMR (CDCl₃) δ 1.14 (t, 3H), 2.1 (m, 2H), 4.78 (t, 1H), 7.0 (s, 1H), 7.2 (d, 1H), 7.41 (d, 1H), 7.63 (d, 1H), 7.74 (d, 1H), 7.89 (s, 1H).

### Step C: Preparation of 2-[(7-bromo-2-naphthalenyl)oxy]-N-(2-methoxy-1,1-dimethylethyl)butanamide

To a mixture of 2-[(7-bromo-2-naphthalenyl)oxy]butanoic acid (i.e. the product of Step B) (309 mg, 1 mmol) and 2-chloro-1-methylpyridinium iodide (281 mg, 1.1 mmol) in dichloromethane (6 mL) at 0° C. was added N,N-diisopropylethylamine (0.7 ml, 4 mmol). The reaction mixture was stirred at ambient temperature for 15 minutes. A solution of 1-methoxy-2-methyl-2-propanamine (114 mg, 1.1 mmol) in dichloromethane (1 mL) was added. The mixture was stirred at room temperature for 18 hours. The reaction mixture was diluted with 10 mL of dichloromethane. The reaction mixture was washed with water (2×15 mL). The organic phase was dried (MgSO₄) and concentrated under reduced pressure. The residue was purified by column chromatography (with solutions of ethyl acetate in hexanes in volume ratios from 10 to 53% as eluents) to give the title compound (0.35 g), a compound of the present invention, as an oil.

¹H NMR (CDCl₃) δ 1.06 (t, 3H), 1.28 (s, 3H), 1.32 (s, 3H), 2.0 (m, 2H), 3.2-3.4 (m, 5H), 4.53 (t, 1H), 6.45 (s, 1H), 7.03 (s, 1H), 7.19 (d, 1H), 7.43 (d, 1H), 7.63 (d, 1H), 7.74 (d, 1H), 7.89 (s, 1H).

### Example 2

### Preparation of 2-[(7-bromo-2-naphthalenyl)oxy]-N-(2-hydroxy-1,1-dimethylethyl)-butanamide (Compound 16)

To a mixture of 2-[(7-bromo-2-naphthalenyl)oxy]butanoic acid (i.e. the product of Step B in Example 1) (927 mg, 3 mmol) and 2-chloro-1-methylpyridinium iodide (843 mg, 3.3 mmol) in dichloromethane (21 mL) at 0° C. was added N,N-diisopropylethylamine (2.1 mL, 12 mmol). The reaction mixture was stirred at ambient temperature for 15 minutes. A solution of 2-amino-2-methyl-1-propanol (294 mg, 3.3 mmol) in dichloromethane (3 mL) was added. The mixture was stirred at room temperature for 3 days. The reaction mixture was diluted with dichloromethane (30 mL). The reaction mixture was washed with water twice (2×45 mL). The organic phase was dried (MgSO₄) and concentrated under reduced pressure. The residue was purified by column chromatography (with solutions of ethyl acetate in hexanes in volume ratios from 10 to 53% as eluents) to give the title compound (0.94 g), a compound of the present invention, as a solid (m.p. 115-116° C.).

¹H NMR (CDCl₃) δ 1.07 (t, 3H), 1.21 (s, 3H), 1.26 (s, 3H), 2.04 (m, 2H), 3.55-3.6 (m, 2H), 4.53 (brs, 1H), 4.6 (t, 1H), 6.42 (s, 1H), 7.04 (s, 1H), 7.19 (d, 1H), 7.43 (d, 1H), 7.63 (d, 1H), 7.74 (d, 1H), 7.89 (s, 1H).

### Example 3

### Preparation of 2-[(7-bromo-2-naphthalenyl)oxy]-N-[2-(methoxymethoxy)-1,1-dimethylethyl]butanamide (Compound 20)

To a solution of 2-[(7-bromo-2-naphthalenyl)oxy]-N (2-hydroxy-1,1-dimethylethyl)-butanamide (i.e. the product of Example 2) (190 mg, 0.5 mmol) in dichloromethane (3 mL) at 0° C. was added a solution of N,N-diisopropylethylamine (84 mg, 0.65 mmol) in dichloromethane (1 mL) and then a solution of bromomethyl methyl ether (81 mg, 0.65 mmole) in dichloromethane (1 mL). The reaction mixture stirred at 0° C. for 21 minutes and then at ambient temperature for 1 hour. Additional N,N-diisopropylethylamine (121 mg, 0.94 mmol) and bromomethyl methyl ether (121 mg, 0.97 mmol) were added. The mixture was stirred at room temperature for 18 hours. To the reaction mixture was then added ethyl acetate (30 mL) and saturated aqueous ammonium chloride solution (20 mL). The organic phase was separated and the aqueous phase was extracted with ethyl acetate (30 mL). The organic phases were combined, washed with brine (40 mL), dried (MgSO₄) and concentrated under reduced pressure. The residue was purified by column chromatography (with solutions of ethyl acetate in hexanes in volume ratios from 4 to 53% as eluents) to give the title compound (197 mg), a compound of the present invention, as an oil.

¹H NMR (CDCl₃) δ 1.04 (t, 3H), 1.29 (s, 3H), 1.33 (s, 3H), 2.0 (m, 2H), 3.24 (s, 3H), 3.39 (d, 1H), 3.48 (d, 1H), 4.49 (m, 3H), 6.5 (s, 1H), 7.03 (s, 1H), 7.17 (d, 1H), 7.4 (d, 1H), 7.6 (d, 1H), 7.7 (d, 1H), 7.84 (s, 1H).

### Example 4

### Preparation of 2-[(7-cyano-2-naphthalenyl)oxy]-N-[2-(methoxymethoxy)-1,1-dimethylethyl]butanamide (Compound 22)

### Step A: Preparation of 2-bromo-N-(2-hydroxy-1,1-dimethylethyl)butanamide

To a mixture of 2-amino-2-methyl-1-propanol (2.67 g, 30 mmol) and triethylamine (5.6 mL, 40 mmol) in tetrahydrofuran (50 mL) at 0° C. was added 2-bromobutyryl bromide (3 mL, 25 mmol) portionwise under nitrogen atomosphere with stirring. The reaction mixture was stirred at ambient temperature for 3 days. The reaction mixture was filtered and the solid was washed with tetrahydrofuran (20 mL). The filtrates were combined and concentrated under reduced pressure. The residue was purified by column chromatography (with solutions of ethyl acetate in hexanes in volume ratios from 11 to 72% as eluents) to give the title compound (4.21 g) as a gummy solid.

¹H NMR (CDCl₃) δ 1.04 (t, 3H), 1.33 (s, 6H), 2.0-2.2 (m, 2H), 3.61 (m, 2H), 4.03 (t, 1H), 4.24 (t, 1H), 6.42 (s, 1H).

### Step B: Preparation of 2-bromo-N-[2-(methoxymethoxy)-1,1-dimethylethyl]-butanamide

To a solution of 2-bromo-N-(2-hydroxy-1,1-dimethylethyl)butanamide (i.e. the product of Step A) (1.19 g, 5 mmol) in dichloromethane (21 mL) at 0° C. was added a solution of bromomethyl methyl ether (2.0 g, 16 mmol) in dichloromethane (7 mL) and then a solution of N,N-diisopropylethylamine (2.78 ml, 16 mmol) in dichloromethane (7 mL) portionwise. The reaction mixture was stirred at 0° C. for 21 minutes and then at room temperature for 18 hours. The reaction mixture was then concentrated under reduced pressure. The resultant residue was purified by column chromatography (with solutions of ethyl acetate in hexanes in volume ratios from 10 to 53% as eluents) to give the title compound (978 mg) as an oil.

¹H NMR (CDCl₃) (1.01 (t, 3H), 1.35 (s, 6H), 2.0-2.2 (m, 2H), 3.35 (s, 3H), 3.5 (s, 2H), 4.19 (t, 1H), 4.62 (s, 2H), 6.5 (s, 1H).

### Step C: Preparation of 2-[(7-cyano-2-naphthalenyl)oxy]-N-[2-(methoxymethoxy)-1,1-dimethylethyl]butanamide

To a solution of 7-hydroxy-2-naphthonitrile (169 mg, 1 mmol) in acetone (8 mL) was added cesium carbonate (586 mg, 1.8 mmol) at room temperature under nitrogen atmosphere with stirring. After the addition, the mixture was stirred at room temperature for 15 minutes and 2-bromo-N-[2-(methoxymethoxy)-1,1-dimethylethyl]butanamide (i.e. the product of Step B) (310 mg, 1.1 mmol) was then added. The mixture was stirred under nitrogen atmosphere at reflux for 5 hours. The reaction mixture was then cooled to room temperature and filtered. The solid was washed with acetone (100 mL). The filtrates were combined and concentrated under reduced pressure. The residue was purified by column chromatography (with solutions of ethyl acetate in hexanes in volume ratios from 10 to 53% as eluents) to give the title compound (0.31 g), a compound of the present invention, as a gummy solid.

¹H NMR (CDCl₃) δ 1.04 (t, 3H), 1.27 (s, 3H), 1.33 (s, 3H), 2.0 (m, 2H), 3.23 (s, 3H), 3.39 (d, 1H), 3.48 (d, 1H), 4.49 (m, 3H), 6.47 (s, 1H), 7.17 (s, 1H), 7.3 (d, 1H), 7.45 (d, 1H), 7.8 (m, 2H), 8.04 (s, 1H).

### Example 5

### Preparation of 2-[(6-bromo-3-quinolinyl)oxy]-N-(1,1-dimethylethyl)butanamide (Compound 37)

### Step A: Preparation of 6-bromo-3-quinolinol

A solution of potassium hydroxide (39.60 g, 707.0 mmol) in water (200 mL) at 50° C. was treated with 5-bromoisatin (10.00 g, 44.2 mmol) in one portion. After 1.5 h the reaction temperature had decreased to 20° C. The resulting mixture was treated with bromopyruvic acid (20.69 g, 123.9 mmol) and stirred at 20° C. for 6 days. The mixture was treated with concentrated aqueous hydrochloric acid to decrease the pH to 4. The resulting precipitate was collected on a coarse frit glass funnel and washed with ethanol then water. The remaining solid was air dried over 18 h affording 13.0 g of a yellow solid, which was suspended in nitrobenzene (200 mL) and heated to 200° C. The mixture was stirred for ten minutes as rapid evolution of carbon dioxide was observed. The solution was filtered hot to remove a tan solid which was discarded. As the filtrate cooled to 20° C. a solid precipitated. The solid was collected, washed with hexanes and allowed to air dry, affording the title compound as a light brown solid (5.30 g).

¹H NMR (DMSO-d₆) δ 8.57 (s, 1H), 8.04 (s, 1H), 7.80 (d, 1H), 7.55 (d, 1H), 7.43 (s, 1H).

### Step B: Preparation of methyl 2-[(6-bromo-3-quinolinyl)oxy]butanoate

A solution of 6-bromo-3-quinolinol (i.e. the product of Step A) (5.0 g, 22.2 mmol) in N,N-dimethylformamide (50 mL) at 25° C. was treated with potassium carbonate (6.13 g, 44.4 mmol) and methyl bromobutyrate (8.04 g, 44.4 mmol). The resulting mixture was stirred for 18 h at 25° C. The reaction mixture was partitioned between ethyl acetate and brine. The combined organic extracts were washed with brine and dried (MgSO₄). The organic phase was concentrated under reduced pressure to leave an orange oil. The resultant oil was purified by column chromatographed on silica gel (with ethyl acetate/hexanes as the eluent). The desired fractions were combined and concentrated to give the title compound as an orange solid (5.0 g).

¹H NMR (CDCl₃) δ 8.73 (m, 1H), 7.90 (d, 1H), 7.86 (d, 1H), 7.63 (d, 1H), 7.17 (d, 1H), 4.71 (t, 1H), 3.78 (s, 3H), 2.09 (m, 2H), 1.13 (t, 3H).

### Step C: Preparation of 2-[(6-bromo-3-quinolinyl)oxy]butanoic acid

A solution of methyl 2-[(6-bromo-3-quinolinyl)oxy]butanoate (i.e. the product of Step B) (2.8 g, 8.64 mmol) in tetrahydrofuran (50 mL) at 25° C. was treated with 50% sodium hydroxide (0.83 g, 10.4 mmol). The resulting mixture was stirred for 18 h at 25° C. The reaction mixture was brought to neutral pH by treatment with 1.25 M hydrochloric acid in methanol to neutral pH. The reaction mixture was concentrated under reduced pressure to leave a solid which was dissolved in dichloromethane (300 mL), filtered and dried (MgSO₄). The dried organic phase was filtered and concentrated to give the title compound as an white solid (2.0 g).

¹H NMR (CDCl₃) δ 8.81 (s, 1H), 8.07 (d, 1H), 7.86 (d, 1H), 7.93 (s, 1H), 7.67 (d, 1H), 7.53 (s, 1H), 2.14 (m, 2H), 1.15 (t, 3H).

### Step D: Preparation of 2-[(6-bromo-3-quinolinyl)oxy]-N-(1,1-dimethylethyl)-butanamide

A solution of 2-[(6-bromo-3-quinolinyl)oxy]butanoic acid (i.e. the product of Step C) (0.5 g, 1.61 mmol) in dichloromethane (50 mL) at 0° C. was treated with 2-chloro-N-methyl-pyridinium iodide (0.41 g, 1.61 mmol) and N,N-diisopropylethylamine (0.83 g, 6.44 mmol). The resulting reaction mixture was stirred at 0° C. for 15 min. The ice bath was then removed and the reaction temperature was allowed to warm to 25° C. Tert-butylamine (0.118 g, 1.61 mmol) was added to the reaction mixture, which was allowed to stir at ambient temperature for 18 h. The crude reaction mixture concentrated and chromatographed (using 10 to 100% ethyl acetate in hexanes as an eluent) to give the title compound, a compound of this invention, as a white solid (0.43 g).

¹H NMR (CDCl₃) δ 8.71 (d, 1H), 7.92 (d, 1H), 7.88 (d, 1H), 7.66 (d, 1H), 7.30 (d, 1H), 4.49 (m, 1H), 2.03 (m, 2H), 1.32 (s, 9H), 1.07 (t, 3H).

### Example 6

### Preparation of 2-[(6-iodo-3-quinolinyl)oxy]-N-(2-methoxy-1,1-dimethylethyl)propanamide (Compound 59)

### Step A: Preparation of 1,1-dimethylethyl-N-(2-methoxy-1,1-dimethylethyl)carbamate

A suspension of N-Boc-2-amino-2-methyl-1-propanol (15 g, 79 mmoles) and tetra-N-butylammonium bisulfate (2.68 g, 7.9 mmol) in toluene (150 mL) was treated with 50% aqueous sodium hydroxide (30 mL) and iodomethane (16.8 g, 118.3 mmol) and stirred at 25° C. for 72 h. The mixture was treated with additional iodomethane (16.8 g, 118 mmol) and stirred for 48 h at 25° C. The reaction mixture was treated with a third portion of iodomethane (16.8 g, 118 mmol) and stirred for 24 h at 25° C. The reaction mixture was then poured into water and extracted with diethyl ether. The organic phase was washed with brine, dried (MgSO₄) and concentrated under reduced pressure to give the title compound as a colorless oil (12.2 g).

¹H NMR (CDCl₃) δ 3.37 (s, 3H), 3.31 (s, 2H), 1.43 (s, 9H), 1.29 (s, 6H).

### Step B: Preparation of 1-methoxy-2-methyl-2-propanamine hydrochloride

A solution of 1,1-dimethylethyl-N-(2-methoxy-1,1-dimethylethyl)carbamate (i.e. the product of Step A) (12.2 g, 60.0 mmol) in ethanol (100 mL) was treated with 6 N aqueous hydrochloric acid (30 mL) and stirred at 50° C. for 48 h. The mixture was cooled to 25° C. and concentrated under reduced pressure to leave a viscous oil. The oil was re-dissolved in ethanol and concentrated once again to an oil. The oil was dissolved a third time in ethanol and concentrated to a constant weight. The resulting oil crystallized upon cooling to afford the title compound as white crystals (8.1 g), which were carried on without further purification.

¹H NMR (CDCl₃) δ 8.35 (s, 3H), 3.42 (s, 5H), 1.45 (s, 6H).

### Step C: Preparation of 2-bromo-N-(2-methoxy-1,1-dimethylethyl)propanamide

A solution of 1-methoxy-2-methyl-2-propanamine hydrochloride (i.e. the product of Step B) (3.0 g, 21.6 mmol) in acetone (25 mL) at 0° C. was treated with triethylamine (6.56 g, 64.8 mmol). The resulting mixture was treated with a solution of 2-bromopropionyl bromide (6.99 g, 32.4 mmol) in acetone (25 mL) while maintaining the reaction temperature below 5° C. When the addition was complete, the ice bath was removed and the reaction mixture was allowed to slowly warm to 25° C. and stir for 18 h. The reaction mixture was concentrated under reduced pressure to leave a white solid. The solid was partitioned between dichloromethane and water, and the organic phase was dried (MgSO₄) and concentrated to give the title compound as an amber oil (4.75 g).

¹H NMR (CDCl₃) δ 4.31 (q, 1H), 3.39 (s, 3H), 3.36 (s, 2H), 1.84 (d, 3H), 1.36 (s, 6H).

### Step D: Preparation of 2-[(6-iodo-3-quinolinyl)oxy]-N-(2-methoxy-1,1-dimethylethyl)propanamide

A suspension of 6-iodo-3-quinolinol (prepared similar to Step A of Example 5) (0.756 g, 2.8 mmol) in acetonitrile (2.0 mL) was treated with 2-bromo-N-(2-methoxy-1,1-dimethylethyl)propanamide (i.e. the product of Step C) (0.740 g, 3.1 mmol) and cesium carbonate (1.36 g, 4.2 mmol). The resulting mixture was subjected to microwave irradiation for a period of 1 h at 125° C. Upon cooling, the mixture was partitioned between dichloromethane and water. The organic phase was dried (MgSO₄) filtered and concentrated. The resultant residue was chromatographed (using 10 to 100% ethyl acetate in hexanes as an eluent), and the appropriate fractions were combined and concentrated to give the title compound, a compound of this invention, as a beige solid (0.235 g).

¹H NMR (CDCl₃) δ 8.69 (d, 1H), 8.11 (d, 1H), 7.83 (d, 1H), 7.77 (d, 1H), 7.27 (d, 1H), 4.64 (q, 1H), 3.29 (q, 2H), 3.27 (s, 3H), 1.64 (s, 3H), 1.34 (s, 3H), 1.34 (s, 3H), 1.30 (s, 3H).

### Example 7

### Preparation of N-(1,1-dimethyl-2-propyn-1-yl)-2-[(6-iodo-3-quinolinyl)oxy]-2-methoxy-acetamide (Compound 63)

### Step A: Preparation of methyl 2-bromo-2-methoxyacetate

A solution of methyl 2-methoxyacetate (20.0 g, 190 mmol) in carbon tetrachloride (200 mL) was treated with N-bromosuccinamide (34.6 g, 200 mmol) and 2-2′-azodiisobutyronitrile (0.10 g, 0.61 mmol). The reaction mixture was stirred at reflux for 1.5 h. The mixture was cooled to 25° C., filtered and dried (MgSO₄). The filtrate was concentrated under reduced pressure to give the title compound (25.6 g), which was carried on without further purification.

¹H NMR (CDCl₃) 6.03 (q, 1H), 3.87 (s, 3H), 3.59 (s, 3H).

### Step B: Preparation of methyl 2-[(6-iodo-3-quinolinyl)oxy]-2-methoxyacetate

A solution of 95% potassium t-butoxide (2.07 g, 184 mmol) in t-butanol (50 mL) was stirred at 25° C. for 0.5 h and then treated with 6-iodo-3-quinolinol (prepared similar to Step A of Example 5) (5.0 g, 184 mmol). The reaction mixture was treated with methyl 2-bromo-2-methoxyacetate (i.e. the product of Step A) (3.37 g, 184 mmol) added dropwise over 0.25 h. After stirring for 18 h at 25° C., the mixture was partitioned between chloroform and brine. The organic phase was washed with water and then dried (MgSO₄). The solution was filtered and concentrated under reduced pressure. The resultant residue was chromatographed (with varying concentrations of ethyl acetate/hexanes as an eluent) to give the title compound as an orange oil (2.33 g).

¹H NMR (CDCl₃) δ 8.78 (d, 1H), 8.15 (d, 1H), 7.85 (d, 1H), 7.78 (d, 1H), 7.60 (d, 1H), 5.63 (s, 1H), 3.87 (s, 3H), 3.56 (s, 3H).

### Step C: Preparation of 2-[(6-iodo-3-quinolinyl)oxy]-2-methoxyacetic acid

A suspension of methyl 2-[(6-iodo-3-quinolinyl)oxy]-2-methoxyacetate (i.e. the product of Step B) (2.3 g, 6.2 mmol) in 1:1 tetrahydrofuran/water (1000 mL) at 0° C. was treated with lithium hydroxide monohydrate (0.284 g, 6.78 mmol). The reaction mixture was stirred at 0° C. for 2.5 h, then allowed to warm to 25° C. and stir for 18 h. The mixture was partitioned between ethyl acetate and brine. The aqueous phase was acidified to pH 3 with 1 N hydrochloric acid and extracted with ethyl acetate. The combined organic extracts were washed with brine, dried (MgSO₄) and concentrated under reduced pressure to give the title compound as a beige powder (1.95 g).

¹H NMR (DMSO d₆) δ 8.76 (d, 1H), 8.39 (d, 1H), 7.89 (d, 1H), 7.86 (d, 1H), 7.77 (d, 1H), 5.84 (s, 1H), 3.46 (s, 3H).

### Step D: Preparation of N-(1,1-dimethyl-2-propyn-1-yl)-2-[(6-iodo-3-quinolinyl)oxy]-2-methoxyacetamide

In a manner similar to that employed in Example 5, Step 4, the subject compound, a compound of the present invention was prepared from 2-[(6-iodo-3-quinolinyl)oxy]-2-methoxyacetic acid (i.e. the product of Step C) as a white solid.

¹H NMR (CDCl₂) δ 8.77 (s, 1H), 8.14 (d, 1H), 7.84 (d, 1H), 7.77 (d, 1H), 7.71 (d, 1H), 6.73 (s, 1H), 5.38 (s, 1H), 3.56 (s, 3H), 2.36 (s, 1H), 1.68 (s, 6H).

### Example 8

### Preparation of N-(1,1-dimethylethyl)-2-methoxy-2-[[6-(1-propyn-1-yl)-3-quinolinyl]oxy]-acetamide (Compound 156)

A solution of 2-[(6-bromo-3-quinolinyl)oxy]-N-(1,1-dimethylethyl)-2-methoxy-acetamide (0.55 g, 1.50 mmoles), (prepared in a similar manner to that described in Example 7, Steps A-D) in toluene (75 mL) at 25° C. was treated with tributyl(1-propynyl)tin (0.59 g, 1.8 mmoles) and tetrakistriphenylphosphine palladium(0) (0.23 g, 0.20 mmoles). The resulting mixture was stirred for 10 h at 100° C. The reaction mixture was poured into water and extracted with ethyl acetate. The organic phase was separated, dried over magnesium sulfate, filtered and concentrated under reduced pressure. The residue was dissolved in dichloromethane, treated with silica gel (5 g) and concentrated to a dry powder. The powder was chromatographed on a silica gel column employing a gradient elution from 20% ethyl acetate/hexanes to 100% ethyl acetate over 15.0 min. The desired fractions were combined and concentrated to give the title compound, a compound of the present invention, as an oil (71.0 mg).

¹H NMR (CDCl₃) δ 8.73 (s, 1H), 7.95 (d, 1H), 7.77 (s, 1H), 7.71 (d, 1H), 7.56 (d, 1H), 6.49 (s, 1H), 5.35 (s, 1H), 3.54 (s, 3H), 2.10 (s, 3H), 1.38 (s, 9H).

### Example 9

### Preparation of N-(2-methoxy-1,1-dimethylethyl)-2-[(7-methyl-2-naphthalenyl)oxy]-butanamide (Compound 159)

A solution of 2-[(7-bromo-2-naphthalenyl)oxy]-N-(2-methoxy-1,1-dimethylethyl)-butanamide (0.59 g, 1.50 mmoles) (prepared as described in Example 1 Steps A-C) in dioxane (5 mL) at 25° C. was treated with a 2 M solution of dimethylzinc in toluene (0.28 g, 3.0 mmoles, 1.5 mL) and bisdiphenylphosphine palladium(II) dichloride (0.031 g, 0.045 mmoles). The resulting mixture was stirred for 10 h at 100° C. The reaction mixture was treated with methanol (5.0 mL) and stirred for 10 minutes. The mixture was then poured into water and extracted with ethyl acetate. The organic phase was separated, dried over magnesium sulfate, filtered and concentrated under reduced pressure. The residue was dissolved in dichloromethane (50.0 mL), treated with silica gel (15 g) and concentrated to a dry powder. The powder was chromatographed on a silica gel column employing a gradient elution from 10% ethyl acetate/hexanes to 90% ethyl acetate/hexanes over 15.0 min. The desired fractions were combined and concentrated to give the title compound, a compound of the present invention, as an oil (0.35 g).

¹H NMR (CDCl₃) δ 7.71 (d, 1H), 7.67 (d, 1H), 7.48 (s, 1H), 7.19 (d, 1H), 7.10 (d, 1H), 7.08 (m, 1H), 6.53 (s, 1H), 4.53 (m, 1H), 3.36 (d, 1H), 3.27 (s, 3H), 3.25 (d, 1H), 2.63 (s, 3H), 1.99 (m, 2H), 1.33 (s, 3H), 1.28 (s, 3H), 1.05 (t, 3H).

### Example 10

### Preparation of 2-[(7-ethyl-2-naphthalenyl)oxy]-N-(2-methoxy-1,1-dimethylethyl)-butanamide (Compound 138)

### Step A: Preparation of 7-methoxy-2-naphthalenyl 1,1,1-trifluoromethanesulfonate

A solution of 7-methoxy-2-naphthol (6.50 g, 37.4 mmoles) in dichloromethane (200 mL) at 0° C. was treated triethylamine (7.56 g, 74.8 mmoles, 10.41 mL). Trifluoromethane-sulfonic anhydride (12.66 g, 40.0 mmoles) was added in a dropwise manner over 30 minutes. The resulting mixture was stirred for 2 h at 0° C., allowed to warm to 25° C. and stirred for 1 h. The reaction mixture was poured into water and extracted with dichloromethane. The organic phase was separated, dried over magnesium sulfate, filtered and concentrated under reduced pressure to give the title compound (10.5 g) as an oil which was carried on without further purification.

¹H NMR (CDCl₃) δ 7.83 (d, 1H), 7.77 (d, 1H), 7.64 (d, 1H), 7.21 (d, 2H), 7.14 (d, 1H), 3.94 (s, 3H).

### Step B: Preparation of 2-ethyl-7-methoxynaphthalene

7-Methoxy-2-naphthalenyl 1,1,1-trifluoromethanesulfonate (i.e. the product of Step A) (5.0 g, 190 mmoles) in a 10% solution of N-methyl-2-pyrrolidone (100 mL) at 25° C. was treated with iron acetylacetonate (0.31 g, 0.8 mmoles). Ethyl magnesium bromide solution (1M in THF) (2.53 g, 19.0 mL, 19 mmoles) was added in one portion. The resulting exotherm to 55° C. began to subside within 10 minutes. The reaction was then cooled to 25° C. and diluted with diethyl ether (100 mL). The reaction mixture was treated with 1N HCl (50 mL) and stirred for 10 minutes. The ether solution was washed with saturated aqueous NaCl solution, dried over magnesium sulfate, filtered and concentrated under reduced pressure. The residue was dissolved in dichloromethane (50 mL), treated with silica gel (15 g) and concentrated to a dry powder. The powder was chromatographed on a silica gel column employing an isocratic elution of 5.0% ethyl acetate/hexanes. The desired fractions were combined and concentrated to give the title compound (1.35 g) as a solid.

¹H NMR (CDCl₃) δ 7.68 (m, 2H), 7.53 (s, 1H), 7.20 (d, 1H), 7.08 (m, 2H), 391 (s, 3H), 2.79 (q, 2H), 1.32 (t, 3H).

### Step C: Preparation of 7-ethyl-2-naphthalenol

A solution of 2-ethyl-7-methoxynaphthalene (i.e. the product of Step B) (1.35 g, 7.26 mmoles) in dichloromethane (50 mL) was cooled to 0° C. and treated with borontribromide (1M in dichloromethane) (2.73 g, 10.89 mmoles, 10.89 mL) in one portion. The mixture was allowed to warm to 25° C. and stir for 18 h. The reaction mixture was cooled to 10° C. and treated with a 2.0% aqueous sodium carbonate solution (25 mL). The phases were separated and the organic phase was washed with saturated aqueous NaCl solution. The organic phase was dried over magnesium sulfate, filtered and concentrated under reduced pressure to give the title compound (1.2 g) as a solid.

¹H NMR (CDCl₃) δ 7.68 (m, 2H), 7.46 (s, 1H), 7.20 (d, 1H), 7.08 (d, 1H), 7.02 (d, 1H), 4.93 (s, 1H), 2.79 (q, 2H), 1.32 (t, 3H).

### Step D: Preparation of methyl 2-[(7-ethyl-2-naphthalenyl)oxy]butanoate

A 25° C. solution of 7-ethyl-2-naphthalenol (i.e. the product of Step C) (5.65 g, 32.8 mmoles) in dioxane (200 mL) was treated with 2-bromobutyrate-methyl ester (11.95 g, 66.0 mmoles) and cesium carbonate (21.5 g, 66.0 mmoles). The resulting mixture was stirred at 100° C. for 18 h. The mixture was cooled to 25° C., poured into a saturated aqueous NaCl solution and extracted with ethyl acetate. The organic phase was dried over magnesium sulfate, filtered and concentrated under reduced pressure. The residue was dissolved in dichloromethane (200 mL), treated with silica gel (40 g) and concentrated to a dry powder. The powder was chromatographed on a silica gel column employing an isocratic elution of 5.0% ethyl acetate/hexanes. The desired fractions were combined and concentrated to give the title compound (5.30 g) as an oil.

¹H NMR (CDCl₃) δ 7.71 (d, 1H), 7.68 (d, 1H), 7.49 (s, 1H), 7.21 (d, 1H), 7.14 (d, 1H), 7.00 (d, 1H), 4.72 (t, 1H), 3.76 (s, 3H), 2.78 (q, 2H), 2.05 (m, 2H), 1.31 (t, 3H), 1.11 (t, 3H).

### Step E: Preparation of 2-[(7-ethyl-2-naphthalenyl)oxy]butanoic acid

Methyl 2-[(7-ethyl-2-naphthalenyl)oxy]butanoate (i.e. the product of Step D) (5.3 g, 19.4 mmole) was converted in a manner similar to that employed in Example 1, Step B to the title compound, a compound of the present invention, as a solid (5.1 g).

¹H NMR (DMSO d₆) δ 7.76 (m, 2H), 7.54 (s, 1H), 7.23 (d, 1H), 7.11 (m, 2H), 4.80 (m, 1H), 2.73 (q, 2H), 1.94 (m, 2H), 1.25 (t, 3H), 1.04 (t, 3H).

### Step F: Preparation of 2-[(7-ethyl-2-naphthalenyl)oxy]-N-(2-methoxy-1,1-dimethylethyl)butanamide

2-[(7-Ethyl-2-naphthalenyl)oxy]butanoic acid (i.e. the product of Step E) (413 mg, 1.6 mmole) was converted in a manner similar to that employed in Example 1, Step C to the title compound, a compound of the present invention, as a solid (0.29 g).

¹H NMR (CDCl₃) δ 7.71 (m, 2H), 7.50 (s, 1H), 7.11 (m, 2H), 4.53 (m, 1H), 3.36 (d, 1H), 3.27 (s, 3H), 3.26 (d, 1H), 2.79 (q, 2H), 200 (m, 2H), 1.33 (s, 3H), 1.31 (t, 3H), 1.28 (s, 3H), 1.06 (t, 3H).

### Example 11

### Preparation of 2-[(7-ethenyl-2-naphthalenyl)oxy]-N-(2-methoxy-1,1-dimethylethyl)-butanamide (Compound 145)

### Step A: Preparation of 7-methoxy-2-naphthalenyl 1,1,2,2,3,3,4,4,4-nonafluoro-1-butanesulfonate

A 0° C. solution of 7-methoxy-2-naphthol (15.0 g, 86.0 mmoles), diisopropyl ethylamine (13.92 g, 108.0 mmoles) and 4-dimethylaminopyridine (1.0 g, 8.6 mmoles) in dichloromethane (250 mL) was treated dropwise with perfluoro-1-butanesulfonylfluoride (32.53 g, 108 mmoles). Upon complete addition, the reaction was allowed to warm to 25° C. and stir for 18 h. The reaction mixture was partitioned between dichloromethane and saturated aqueous NaCl solution. The organic phase was dried over magnesium sulfate and filtered. The dried dichloromethane solution was treated with silica gel (50 g) and concentrated to a dry powder. The powder was chromatographed on a silica gel column employing an isocratic elution of 10.0% ethyl acetate/hexanes. The desired fractions were combined and concentrated to give the title compound (28.0 g) as a solid.

¹H NMR (CDCl₃) δ 7.83 (d, 1H), 7.77 (d, 1H), 7.65 (d, 1H), 7.21 (m, 2H), 3.94 (s, 3H).

### Step B: Preparation of 7-hydroxy-2-naphthalenyl 1,1,2,2,3,3,4,4,4-nonafluoro-1-butanesulfonate

7-Methoxy-2-naphthalenyl 1,1,2,2,3,3,4,4,4-nonafluoro-1-butanesulfonate (i.e. the product of Step A) (10.0 g, 25.5 mmole) was converted in a manner similar to that employed in Example 10, Step C to the title compound, a compound of the present invention, as a solid (8.0 g).

¹H NMR (CDCl₃) δ 7.84 (d, 1H), 7.80 (d, 1H), 7.59 (d, 1H), 7.22 (d, 1H), 7.17 (m, 2-1H), 5.10 (s, 1H).

### Step C: Preparation of 7-[1-[[(2-methoxy-1,1-dimethylethyl)amino]carbonyl]-propoxy]-2-naphthalenyl 1,1,2,2,3,3,4,4,4-nonafluoro-1-butanesulfonate

A mixture of 7-hydroxy-2-naphthalenyl 1,1,2,2,3,3,4,4,4-nonafluoro-1-butanesulfonate (i.e. the product of Step B) (1.89 g, 5.0 mmoles), 2-bromo-N-(2-methoxy-1,1-dimethylethyl)butanamide (1.50 g, 5.94 mmoles) and cesium carbonate (3.87 g, 12.0 mmoles) in dioxane (100 mL) was stirred at 100° C. for 18 h. The mixture was cooled to 25° C., poured into saturated aqueous NaCl solution and extracted with ethyl acetate. The organic phase was dried over magnesium sulfate, filtered and concentrated under reduced pressure. The residue was dissolved in dichloromethane (200 mL), treated with silica gel (25 g) and concentrated to a dry powder. The powder was chromatographed on a silica gel column employing a gradient elution from 10.0% to 50% ethyl acetate/hexanes. The desired fractions were combined and concentrated to give the title compound (1.40 g) as an oil.

¹H NMR (CDCl₃) δ 7.85 (d, 1H), 7.82 (d, 1H), 7.62 (d, 1H), 7.26 (d, 2H), 7.18 (d, 1H), 6.43 (s, 1H), 4.53 (m, 1H), 3.30 (q, 2H), 3.25 (s, 3H), 2.02 (m, 2H), 1.32 (s, 3H), 1.28 (s, 1H), 1.07 (t, 3H).

### Step D: Preparation of 2-[(7-ethenyl-2-naphthalenyl)oxy]-N-(2-methoxy-1,1-dimethylethyl)butanamide

A mixture of 7-[1-[[(2-methoxy-1,1-dimethylethyl)amino]carbonyl]propoxy]-2-naphthalenyl 1,1,2,2,3,3,4,4,4-nonafluoro-1-butanesulfonate (i.e. the product of Step C) (1.0 g, 1.63 mmoles), bis-diphenylphosphinepalladium(II) dichloride (0.057 g, 0.082 mmoles), lithium chloride (0.55 g, 13.0 mmoles) and tributyl(vinyl)tin (0.65 g, 2.04 mmoles) in dimethylformamide (75 mL) was stirred at 100° C. for 18 h. The mixture was cooled to 25° C., poured into saturated aqueous NaCl solution and extracted with ethyl acetate. The organic phase was washed with saturated aqueous NaCl solution, dried over magnesium sulfate, filtered and concentrated under reduced pressure. The residue was dissolved in dichloromethane (200 mL), treated with silica gel (15 g) and concentrated to a dry powder. The powder was chromatographed on a silica gel column employing a gradient elution from 100% hexanes to 50% ethyl acetate/hexanes over 15 minutes. The desired fractions were combined and concentrated to give the title compound, a compound of the present invention, as an oil (0.220 g).

¹H NMR (CDCl₃) δ 7.72 (m, 2H), 7.63 (s, 1H), 7.51 (d, 1H), 7.14 (m, 2H), 6.85 (m, 1H), 6.51 (s, 1H), 5.86 (d, 1H), 5.33 (d, 1H), 4.53 (m, 1H), 3.35 (d, 1H), 3.26 (s, 3H), 3.25 (d, 1H), 2.01 (m, 2H), 1.33 (s, 3H), 1.28 (s, 3H), 1.06 (t, 3H).

### Example 12

### Preparation of 2-[(7-ethynyl-2-naphthalenyl)oxy]-N-(2-methoxy-1,1-dimethylethyl)-butanamide (Compound 97)

Step A: Preparation of 1,1′-(2,7-naphthalenediyl)bis(1,1,1-trifluoromethanesulfonate)

To 200 mL of dichloromethane was added 2,7-dihydroxynaphthalene (5.4 g, 34 mmol) and pyridine (9.06 mL, 112 mmole) at room temperature. The mixture was cooled to 0° C. under a nitrogen atomosphere with stirring using an ice/acetone bath. To this mixture at 0° C. was then added trifluoromethanesulfonic anhydride (13.24 mL, 78.6 mmole) portionwise. After the addition, the mixture was stirred at ambient temperature for 2.5 hours. Dichloromethane (200 mL) and water (400 mL) were then added. The mixture was acicified with 1N hydrochloric acid aqueous solution to pH 3 with stirring. The organic phase was separated, dried over MgSO₄ and concentrated under reduced pressure. The residue was purified by column chromatography (over silica gel with ethyl acetate and hexanes in volume ratios from 5 to 40% as eluents) to give 14 g of the crude product. The crude product was then trituated with hexane (350 mL) and the solid was collected by filtration to give the title compound (3.5 g) as a solid. The solid which precipitated from the mother liquor was also collected by filtration to give another 424 mg of the title compound. The filtrate was the concentrated to about 100 mL in volume and the precipitate was collected by filtration to give another 8.1 g of the title compound.

¹H NMR (CDCl₃) 7.49 (d, 2H), 7.81 (s, 2H), 8.01 (d, 2H).

### Step B: Preparation of 7-hydroxy-2-naphthalenyl 1,1,1-trifluoromethanesulfonate

To a solution of 1,1′-(2,7-naphthalenediyl)bis(1,1,1-trifluoromethanesulfonate) (i.e. the product of Step A) (3.88 g, 9.2 mmol) in tetrahydrofuran (50 mL) at 0° C. was added potassium t-butoxide (1.35 g, 12 mmol) with stirring and ice/acetone bath cooling. The mixture was stirred at 0° C. for 1 h and then allowed to warm to room temperature and stir at room temperature overnight. The reaction mixture was then cooled to 0° C. and additional potassium t-butoxide (0.9 g, 8 mmol) was added with stirring and ice/acetone bath cooling. The reaction mixture was stirred at ambient temperature for 2 hours. Ethyl acetate (350 mL), saturated aqueous NaCl solution (350 mL) and 1N hydrochloric acid aqueous solution (20 mL) were added to the reaction mixture and the organic phase was separated, dried over MgSO₄ and concentrated. The residue was purified by column chromatography (over silica gel with ethyl acetate and hexanes in volume ratios from 5 to 53% as eluents) to give the title compound (2 g) as an oil.

¹H NMR (CDCl₃) δ 5.43 (s, 1H), 7.18 (m, 3H), 7.57 (s, 1H), 7.82 (m, 2H).

### Step C: Preparation of methyl 2-[[7-[[(trifluoromethyl)sulfonyl]oxy]-2-naphthalenyl]-oxy]butanoate

7-Hydroxy-2-naphthalenyl 1,1,1-trifluoromethanesulfonate (i.e. the product of Step B) (642 mg, 2.2 mmol) was dissolved in acetone (21 mL) and treated with cesium carbonate (1.81 g, 5.5 mmole) at room temperature under a nitrogen atomosphere with stirring. After the addition, the mixture was stirred at room temperature for 5 min and then treated with methyl-2-bromobutyrate (5.4 g, 30 mmol). The mixture was refluxed for 2 hours and then cooled to room temperature and filtered. The solid was washed with acetone (7 mL) and the filtrates were combined and concentrated under reduced pressure. The residue was purified by column chromatography (over silica gel with ethyl acetate and hexanes in volume ratios from 10 to 40% as eluents) to give the title compound (0.86 g) as a solid.

¹H NMR (CDCl₃) δ 1.12 (t, 3H), 2.05 (m, 2H), 3.78 (s, 3H), 4.75 (t, 1H), 7.03 (s, 1H), 7.2-7.3 (m, 2H), 7.6 (s, 1H), 7.79-7.84 (m, 2H).

### Step D: Preparation of methyl 2-[[7-[2-(trimethylsilyl)ethynyl]-2-naphthalenyl]-oxy]butanoate

Methyl 2-[[7-[[(trifluoromethyl)sulfonyl]oxy]-2-naphthalenyl]-oxy]butanoate (i.e. the product of Step C) (0.86 g, 2.19 mmol) was dissolved in tetrahydrofuran (21 mL) and treated with bis(triphenylphosphine)palladium(II) dichloride (212 mg, 0.3 mmole) and copper (I) iodide (114 mg, 0.6 mmole) under nitrogen atomosphere with stirring. After the addition, the reaction mixture was purged with nitrogen for 10 minutes treated sequentially with triethylamine (7.5 mL) and ethynyltrimethylsilane (787 mg, 8 mmole). The mixture was stirred at room temperature under nitrogen atomosphere overnight and was then concentrated under reduced pressure. The residue was purified by column chromatography (over silica gel with ethyl acetate and hexanes in volume ratios from 5 to 40% as eluents) to give the title compound (730 mg) as an oil.

¹H NMR (CDCl₃) δ 0.28 (s, 9H), 1.12 (t, 3H), 2.05 (m, 2H), 3.76 (s, 3H), 4.72 (t, 1H), 6.96 (s, 1H), 7.19 (d, 1H), 7.37 (d, 1H), 7.67-7.72 (m, 2H), 7.84 (s, 1H).

### Step E: Preparation of 2-[(7-ethynyl-2-naphthalenyl)oxy]butanoic acid

To a solution of methyl 2-[[7-[2-(trimethylsilyl)ethynyl]-2-naphthalenyl]-oxy]butanoate (i.e. the product of Step D) (730 mg, 2.15 mmol) in tetrahydrofuran (5 mL) was added 1 N sodium hydroxide aqueous solution (6 mL, 6 mmol) under nitrogen atomosphere at room temperature with stirring. The mixture was stirred at room temperature for 3 h and then ethyl acetate (35 mL) and 1N hydrochloric acid aqueous solution (6.5 mL) were added. The organic phase was separated, washed with saturated aqueous NaCl solution (30 mL), dried over MgSO₄ and concentrated under reduced pressure to give the title compound (0.5 g) as a solid.

¹H NMR (CDCl₃) δ 1.13 (t, 3H), 2.1 (m, 2H), 3.13 (s, 1H), 4.78 (t, 1H), 7.03 (s, 1H), 7.22 (d, 1H), 7.4 (d, 1H), 7.7-7.78 (m, 2-1H), 7.9 (s, 1H).

### Step F: Preparation of 2-[(7-ethynyl-2-naphthalenyl)oxy]-N-(2-methoxy-1,1-dimethylethyl)-butanamide

To a mixture of 2-[(7-ethynyl-2-naphthalenyl)oxy]butanoic acid (i.e. the product of Step E) (500 mg, 1.97 mmol) and 2-chloro-1-methylpyridium iodide (553 mg, 2.17 mmol) in dichloromethane (21 mL) at 0° C. under nitrogen atomosphere was added N,N-diisopropylethylamine (1.72 mL, 9.9 mmole). The reaction mixture was stirred at ambient temperature for 15 minutes and then treated with 1-methoxy-2-methyl-2-propanamine hydrochloride (i.e. the product of Example 6, Step B) (304 mg, 2.18 mmol). The reaction mixture was stirred at room temperature for 4 hours and then diluted with 50 mL of dichloromethane. The reaction mixture was washed with water twice (60 mL each) and the organic phase was separated, dried over MgSO₄ and concentrated under reduced pressure. The residue was purified by column chromatography (over silica gel with ethyl acetate and hexanes in volume ratios from 5 to 53% as eluents) to give the title compound, a compound of the present invention, as an oil (564 mg).

¹H NMR (CDCl₃) δ 1.06 (t, 3H), 1.28 (s, 3H), 1.32 (s, 3H), 2.0 (m, 2H), 3.14 (s, 1H), 3.2-3.4 (m, 5H), 4.53 (t, 1H), 6.47 (s, 1H), 7.12 (s, 1H), 7.19 (d, 1H), 7.41 (d, 1H), 7.7-7.78 (m, 2H), 7.89 (s, 1H).

### Example 13

### Preparation of 2-[(7-ethynyl-2-naphthalenyl)oxy]-2-methoxy-N-(2-methoxy-1,1-dimethylethyl)acetamide (Compound 106)

### Step A: Preparation of methyl 2-methoxy-2-[[7-[[(trifluoromethyl)sulfonyl]oxy]-2-naphthalenyl]oxy]acetate

To a solution of 1,1′-(2,7-naphthalenediyl)bis(1,1,1-trifluoromethanesulfonate) (i.e. the product of Example 12, Step A) (2.12 g, 5 mmol) in tetrahydrofuran (21 mL) at 0° C. under nitrogen atomosphere was added potassium t-butoxide (1.18 g, 10.5 mmol) with stirring and ice/acetone bath cooling. The mixture was stirred at 0° C. for 30 min and at ambient temperature for 1 hour. Methyl bromomethoxyacetate (i.e. the product of Example 7, Step A) (1.3 g, 7.1 mmole) was added with stirring. The reaction mixture was stirred at room temperature for another hour. Ethyl acetate (152 mL) and saturated aqueous NaCl solution (152 mL) were added. The organic phase was separated, washed with water (180 mL), dried over MgSO₄ and concentrated. The residue was purified by column chromatography (over silica gel with ethyl acetate and hexanes in volume ratios from 10 to 53% as eluents) to give 1.65 g of a crude product which was further purified by column chromatography (over silica gel with dichloromethane and hexanes in volume ratios from 35 to 65% as eluents) to give the title compound (1.53 g) as a solid.

¹H NMR (CDCl₃) δ 3.56 (s, 3H), 3.87 (s, 3H), 5.66 (s, 1H), 7.28 (d, 1H), 7.37 (d, 1H), 7.44 (s, 1H), 7.65 (s, 1H), 7.8-7.9 (m, 2H).

### Step B: Preparation of methyl 2-methoxy-2-[[7-[2-(trimethylsilyl)ethynyl]-2-naphthalenyl]oxy]acetate

Methyl 2-methoxy-2-[[7-[[(trifluoromethyl)sulfonyl]oxy]-2-naphthalenyl]oxy]acetate (i.e. the product of Step A) (1.35 g, 3.42 mmol), tetrahydrofuran (35 mL), bis(triphenylphosphine)palladium(II) dichloride (0.33 g, 0.47 mmole) and copper (I) iodide (178 mg, 0.94 mmole) were combined at room temperature under a nitrogen atomosphere. The reation mixture was purged with nitrogen for another 10 minutes and then treated sequentially with triethylamine (15 mL) and ethynyltrimethylsilane (1.23 g, 12.50 mmole). The mixture was stirred at room temperature under a nitrogen atomosphere for 7 hours and then concentrated under reduced pressure. The residue was purified by column chromatography (over silica gel with ethyl acetate and hexanes in volume ratios from 10 to 40% as eluents) to give a crude product (1.27 g) which was further purified (over silica gel with dichloromethane and hexanes in volume ratios from 35 to 65% as eluents) to give the title compound (1.15 g) as a gummy oil.

¹H NMR (CDCl₃) δ 0.28 (s, 9H), 3.55 (s, 3H), 3.86 (s, 3H), 5.62 (s, 1H), 7.28 (d, 1H), 7.35 (s, 1H), 7.41 (d, 1H), 7.69-7.78 (m, 2H), 7.91 (s, 1H).

### Step C: Preparation of 2-[(7-ethynyl-2-naphthalenyl)oxy]-2-methoxyacetic acid

To a solution of methyl 2-methoxy-2-[[7-[2-(trimethylsilyl)ethynyl]-2-naphthalenyl]oxy]acetate (i.e. the product of Step B) (1.15 g, 3.4 mmol) in a mixture of tetrahydrofuran (50 mL) and water (50 mL) at 0° C. under nitrogen atomosphere was added lithium hydroxide monohydrate (0.34 g, 8 mmole) with stirring. The mixture was stirred under a nitrogen atomosphere at 0° C. for 1 hour and then at ambient temperature for 3 h. Ethyl acetate (105 mL) and 1N hydrochloric acid aqueous solution (10 mL) were added. The organic phase was separated, washed with saturated aqueous NaCl solution (100 mL), dried over MgSO₄ and concentrated under reduced pressure to give the title compound (0.85 g) as a gummy solid.

¹H NMR (CDCl₃) δ 3.15 (s, 1H), 3.57 (s, 3H), 5.68 (s, 1H), 7.3 (d, 1H), 7.39-7.45 (m, 2H), 7.7-7.8 (m, 2H), 7.92 (s, 1H), 10.7 (brs 1H).

### Step D: Preparation of 2-[(7-ethynyl-2-naphthalenyl)oxy]-2-methoxy-N-(2-methoxy-1,1-dimethylethyl)acetamide

To a mixture of 2-[(7-ethynyl-2-naphthalenyl)oxy]-2-methoxyacetic acid (i.e. the product of Step C) (0.85 g, 3.3 mmol) and 2-chloro-1-methylpyridium iodide (935 mg, 3.65 mmol) in dichloromethane (35 mL) at 0° C. under nitrogen atomosphere was added N,N-diisopropylethylamine (2.9 mL, 16.5 mmole) with stirring and ice-bath cooling. The reaction mixture was stirred at ambient temperature for 15 minutes and then treated with 1-methoxy-2-methyl-2-propanamine hydrochloride (i.e. the product of Example 6, Step B) (510 mg, 3.65 mmol). The mixture was stirred at room temperature for 3 hours and then diluted with 91 mL of dichloromethane. The reaction mixture was washed with water twice (91 mL each) and the organic phase was separated, dried over MgSO₄ and concentrated under reduced pressure. The residue was purified by column chromatography (over silica gel with ethyl acetate and hexanes in volume ratios from 10 to 53% as eluents) to give the title compound, a compound of the present invention, as an oil which later solidified (848 mg), mp 91-92° C.

¹H NMR (CDCl₃) 1.38 (s, 3H), 1.4 (s, 3H), 3.14 (s, 1H), 3.35-3.43 (m, 5H), 3.51 (s, 3H), 5.4 (s, 1H), 6.8 (s, 1H), 7.3 (d, 1H), 7.43 (m, 2H), 7.7-7.78 (m, 2H), 7.92 (s, 1H).

### Example 14

### Preparation of 2-[(7-bromo-2-naphthalenyl)oxy]-2-methoxy-N-(2-methoxy-1,1-dimethylethyl)acetamide (Compound 90)

### Step A: Preparation of methyl 2-[(7-bromo-2-naphthalenyl)-oxy]-2-methoxyacetate

To a solution of potassium t-butoxide (2.07 g, 18.4 mmol) in t-butanol (50 mL) at room temperature was added 7-bromo-2-naphthol (4.1 g, 18.4 mmol) with stirring. The mixture was stirred at room temperature for 5 min and then methyl bromomethoxyacetate (i.e. the product of Example 7, Step A) (3.37 g, 18.4 mmole) was added portionwise while keeping the temperature below 30° C. The reaction mixture was stirred at ambient temperature overnight and then treated with ethyl acetate (350 mL) and saturated aqueous NaCl solution (350 mL). The organic phase was separated, washed with water (150 mL), dried over MgSO₄ and concentrated. The residue was purified by column chromatography (over silica gel with dicholormethane and hexanes in volume ratio of 50% as eluent) to give the title compound (4.5 g) as an oil.

¹H NMR (CDCl₃) δ 3.55 (s, 3H), 3.86 (s, 3H), 5.63 (s, 1H), 7.28-7.35 (m, 2H), 7.45 (d, 1H), 7.65 (d, 1H), 7.77 (d, 1H), 7.92 (s, 1H).

### Step B: Preparation of 2-[(7-bromo-2-naphthalenyl)-oxy]-2-methoxyacetic acid

To a solution of methyl 2-[(7-bromo-2-naphthalenyl)-oxy]-2-methoxyacetate (i.e. the product of Step A) (4.5 g, 13.8 mmol) in a mixture of tetrahydrofuran (450 mL) and water (450 mL) at 0° C. was added lithium hydroxide monohydrate (672 mg, 16 mmole) with stirring. The mixture was stirred at 0° C. for 2.5 hours and then at ambient temperature overnight. Ethyl acetate (500 mL) was added and the two phases were separated. The aqueous phase was acidified with 1N hydrochloric acid (10 mL) and extracted with ethyl acetate (500 mL). The ethyl acetate extract was dried over MgSO₄ and concentrated under reduced pressure to give the title compound (3.9 g).

¹H NMR (CDCl₃) δ 3.58 (s, 3H), 5.67 (s, 1H), 7.29 (d, 1H), 7.36 (s, 1H), 7.47 (d, 1H), 7.65 (d, 1H), 7.77 (d, 1H), 7.92 (s, 1H).

### Step C: Preparation of 2-[(7-bromo-2-naphthalenyl)oxy]-2-methoxy-N-(2-methoxy-1,1-dimethylethyl)acetamide

To a mixture of 2-[(7-bromo-2-naphthalenyl)-oxy]-2-methoxyacetic acid (i.e. the product of Step B) (3.9 g, 12.54 mmol) and 2-chloro-1-methylpyridium ioide (3.52 g, 13.74 mmol) in dichloromethane (75 mL) at 0° C. was added N,N-diisopropylethylamine (11.2 mL, 64.2 mmole) with stirring and ice-bath cooling. The reaction mixture was allowed to warm and stir at ambient temperature for 15 minutes. 1-Methoxy-2-methyl-2-propanamine hydrochloride (i.e. the product of Example 6, Step B) (1.94 g, 13.8 mmol) was added and the mixture was stirred at room temperature for 3 days. The reaction mixture was then diluted with 150 mL of dichloromethane and washed with water twice (175 mL each). The organic phase was dried over magnesium sulfate and was concentrated under reduced pressure. The residue was purified by column chromatography (over silica gel with ethyl acetate and hexanes in volume ratios from 5 to 53% as eluents) to give the title compound, a compound of the present invention, as a solid (4.4 g), mp 65-67° C.

¹H NMR (CDCl₃) δ 1.38 (s, 3H), 1.4 (s, 3H), 3.35-3.43 (m, 5H), 3.5 (s, 3H), 5.39 (s, 1H), 6.8 (s, 1H), 7.3 (d, 1H), 7.39 (s, 1H), 7.43 (d, 1H), 7.65 (d, 1H), 7.74 (d, 1H), 7.92 (s, 1H).

### Example 15

### Preparation of 2-[(7-ethynyl-2-naphthalenyl)oxy]-N-(2-hydroxy-1,1-dimethylethyl)-butanamide (Compound 169)

To a mixture of 2-[(7-ethynyl-2-naphthalenyl)oxy]butanoic acid (i.e. the product of Example 12, Step E) (0.76 g, 3 mmol) and 2-chloro-1-methylpyridium ioide (843 mg, 3.3 mmol) in dichloromethane (21 mL) at 0° C. under nitrogen atomosphere was added N,N-diisopropylethylamine (2.1 mL, 12 mmole). The reaction mixture was stirred at ambient temperature for 15 min and then a solution of 2-amino-2-methyl-1-propanol (294 mg, 3.3 mmol) in dichloromethane (3 mL) was added. The reaction mixture was stirred at room temperature for 4 and then diluted with 35 mL of dichloromethane. The reaction mixture was washed with water twice (50 mL each). The organic phase was dried over magnesium sulfate and was concentrated under reduced pressure. The residue was purified by column chromatography (over silica gel with ethyl acetate and hexanes in volume ratios from 10 to 65% as eluents) to give the title compound, a compound of the present invention, as a solid, (375 mg), mp 81-82° C.

¹H NMR (CDCl₃) δ 1.07 (t, 3H), 1.20 (s, 3H), 1.26 (s, 3H), 2.03 (m, 2H), 3.16 (s, 1H), 3.58 (m, 2H), 4.53 (t, 1H), 4.6 (t, 1H), 6.42 (s, 1H), 7.09 (s, 1H), 7.19 (d, 1H), 7.43 (d, 1H), 7.7-7.8 (m, 2H), 7.9 (s, 1H).

### Example 16

### Preparation of 2-[(7-ethynyl-2-naphthalenyl)oxy]-N-[2-(methoxymethoxy)-1,1-dimethylethyl]butanamide (Compound 112)

To a solution of 2-[(7-ethynyl-2-naphthalenyl)oxy]-N-(2-hydroxy-1-dimethylethyl)-butanamide (i.e. the product of Example 15) (325 mg, 1.0 mmol) in dichloromethane (10 mL) at 0° C. under nitrogen atomosphere was added a solution of N,N-diisopropylethylamine (645 mg, 5 mmole) in dichloromethane (5 mL) and then a solution of bromomethyl methyl ether (625 mg, 5 mmole) in dichloromethane (5 mL). The reaction mixture stirred at 0° C. for 21 minutes and then at ambient temperature for 1 hour. The reaction mixture was then diluted with ethyl acetate (60 mL) and saturated aqueous ammonium chloride solution (40 mL). The organic phase was separated and the aqueous phase was extracted with ethyl acetate (50 mL). The organic phases were combined, washed with saturated aqueous NaCl solution (80 mL), dried over magnesium sulfate and concentrated under reduced pressure. The residue was purified by column chromatography (over silica gel with ethyl acetate and hexanes in volume ratios from 4 to 53% as eluents) to give the title compound, a compound of the present invention, as an oil (0.23 g).

¹H NMR (CDCl₃) δ 1.06 (t, 3H), 1.31 (s, 3H), 1.36 (s, 3H), 2.0 (m, 2H), 3.15 (s, 1H), 3.26 (s, 3H), 3.42 (d, 1H), 3.45 (d, 1H), 4.51 (m, 3H), 6.55 (s, 1H), 7.1 (s, 1H), 7.19 (d, 1H), 7.41 (d, 1H), 7.7-7.78 (m, 2H), 7.89 (s, 1H).

### Example 17

### Preparation of 2-methoxy-N-(2-methoxy-1,1-dimethylethyl)-2-[[7-(1-propyn-1-yl)-2-naphthalenyl]oxy]acetamide (Compound 108)

To a solution of 2-[(7-bromo-2-naphthalenyl)oxy]-2-methoxy-N-(2-methoxy-1,1-dimethylethyl)acetamide (i.e. the product of Example 14, Step C) (0.6 g, 1.5 mmol) in toluene (15 mL) at room temperature under nitrogen atomosphere was added tributyl(1-propynyl)tin (593 mg, 1.8 mmole) and tetrakis(triphenylphosphine)paladium (235 mg, 0.2 mmole). The reaction mixture was stirred at reflux under nitrogen atomosphere overnight. The reaction mixture was cooled to room temperature and filtered through Celite® filter aid. The Celite® pad was washed with toluene and the filtrates were combined and concentrated under reduced pressure. The residue was purified by column chromatography (over silica gel with solutions of ethyl acetate and hexanes in volume ratios from 10 to 53% as eluents) to give the title compound, a compound of the present invention, as an oil (182 mg).

¹H NMR (CDCl₃) δ 1.38 (s, 3H), 1.4 (s, 3H), 2.1 (s, 1H), 3.35-3.45 (m, 5H), 3.5 (s, 3H), 5.39 (s, 1H), 6.81 (s, 1H), 7.21-7.4 (m, 3H), 7.65-7.78 (m, 2H), 7.8 (s, 1H).

### Example 18

### Preparation of 2-[(7-iodo-2-naphthalenylyl)oxy]-2-methoxy-N-(2-methoxy-1,1-dimethylethyl)acetamide (Compound 103)

### Step A: Preparation of 2-bromo-7-[[(1,1-dimethylethyl)dimethylsilyl]oxy]naphthalene

To a solution of 7-bromo-2-naphthol (2.23 g, 10 mmol) in dichloromethane (21 mL) at 0° C. was added imidazole (1.5 g, 22 mmole) and t-butyldimethylsilylchloride (1.66 g, 11 mmole). The reaction mixture was stirred at room temperature overnight. The reaction mixture was diluted with dichloromethane (70 mL) and the resulting mixture was washed with saturated aqueous NaHCO₃ solution twice (100 mL each). The organic phase was separated, dried over magnesium sulfate and concentrated under reduced pressure. The residue was purified by column chromatography (over silica gel with ethyl acetate and hexanes in volume ratios from 10 to 40% as eluents) to give the title compound (3.3 g) as an oil.

¹H NMR (CDCl₃) δ 0.25 (s, 6H), 1.02 (s, 9H), 7.08 (m, 2H), 7.4 (d, 1H), 7.6 (d, 1H), 7.7 (d, 1H), 7.85 (s, 1H).

### Step B: Preparation of 2-[[(1,1-dimethylethyl)dimethylsilyl]oxy]-7-iodonaphthalene

To a solution of 2-bromo-7-[[(1,1-dimethylethyl)dimethylsilyl]oxy]naphthalene (i.e. the product of Step A) (1.35 g, 4.0 mmol) in tetrahydrofuran (21 mL) at −78° C. under nitrogen atomosphere was added n-butyllithium solution in hexanes (2.0 ml of 2.5 M, 5.0 mmole) dropwise with dry ice/acetone bath cooling while keeping the temperature below −65° C. The reaction mixture was stirred at −78° C. for 0.5 hour. Iodine (1.27 g, 5.0 mmole) was added to the reaction mixture portionwise and then the reaction mixture was stirred at room temperature overnight. The reaction mixture was poured into a mixture of ethyl acetate (70 mL) and water (70 mL). The organic phase was separated, dried over magnesium sulfate and concentrated under reduced pressure. The residue was purified by column chromatography (over silica gel with ethyl acetate and hexanes in volume ratios from 0.5-10% as eluents) to give the title compound (1.13 g) as an oil.

¹H NMR (CDCl₂) δ 0.24 (s, 6H), 1.01 (s, 9H), 7.06 (m, 2H), 7.49 (d, 1H), 7.58 (d, 1H), 7.67 (d, 1H), 8.09 (s, 1H).

### Step C: Preparation of 7-iodo-2-naphthalenol

To a solution of 2-[[(1,1-dimethylethyl)dimethylsilyl]oxy]-7-iodonaphthalene (i.e. the product of Step B) (1.08 g, 2.81 mmol) in tetrahydrofuran (21 mL) at room temperature was added 1.0 M tetrabutylammonium fluoride solution in tetrahydrofuran (3.1 ml, 3.1 mmole) portionwise. The reaction mixture was stirred at room temperature for 2 hours and then diluted with ethyl acetate (210 mL) and water (210 mL). The organic phase was separated, washed with saturated aqueous NH₄Cl solution twice (110 mL each), water (110 mL) and then dried over magnesium sulfate and concentrated under reduced pressure to give the title compound (743 mg) as a solid.

¹H NMR (CDCl₃) δ 5.02 (brs, 1H), 7.03 (s, 1H), 7.1 (d, 1H), 7.49 (d, 1H), 7.56 (d, 1H), 7.72 (d, 1H), 8.09 (s, 1H).

### Step D: Preparation of methyl 2-[(7-iodo-2-naphthalenyl)oxy]-2-methoxyacetate

To a solution of potassium t-butoxide (622 mg, 5.56 mmol) in t-butanol (18 mL) at room temperature was added 7-iodo-2-naphthalenol (i.e. the product of Step C) (1.5 g, 5.56 mmol)) with stirring. The mixture was stirred at room temperature for 5 min and then methyl bromomethoxyacetate (i.e. the product of Example 7, Step A) (1.02 g, 5.56 mmole) was added portionwise with stirring while keeping the temperature below 30° C. The reaction mixture was stirred at room temperature overnight. Chloroform (112 mL) and saturated aqueous NaCl solution (112 mL) were added. The organic phase was separated, washed with water (50 mL), dried over MgSO₄ and concentrated. The residue was purified by column chromatography (over silica gel with ethyl acetate and hexanes in volume ratios from 5 to 10% as eluents) to give the title compound (1.44 mg) as an oil.

¹H NMR (CDCl₃) δ 3.54 (s, 3H), 3.85 (s, 3H), 5.63 (s, 1H), 7.22-7.30 (m, 2H), 7.48 (d, 1H), 7.6 (d, 1H), 7.72 (d, 1H), 8.14 (s, 1H).

### Step E: Preparation of 2-[(7-iodo-2-naphthalenyl)oxy]-2-methoxyacetic acid

To a solution of methyl 2-[(7-iodo-2-naphthalenyl)oxy]-2-methoxyacetate (i.e. the product of Step D) (1.436 g, 3.86 mmol) in a mixture of tetrahydrofuran (150 mL) and water (150 mL) at 0° C. was added lithium hydroxide monohydrate (190 mg, 4.53 mmole) with stirring. The mixture was stirred at 0° C. for 2.5 hours and then at ambient temperature overnight. The reaction mixture was treated with ethyl acetate (197 mL) and 1N hydrochloric acid solution (5 mL). The aqueous phase was extracted with ethyl acetate (500 mL). The combined organic phases were dried over MgSO₄ and concentrated under reduced pressure to give the title compound (1.4 g).

¹H NMR (CDCl₃) δ 3.56 (s, 3H), 5.66 (s, 1H), 7.22-7.37 (m, 2H), 7.48 (d, 1H), 7.61 (d, 1H), 7.72 (d, 1H), 8.13 (s, 1H).

### Step F: Preparation of 2-[(7-iodo-2-naphthalenylyloxy]-2-methoxy-N-(2-methoxy-1,1-dimethylethyl)acetamide

To a mixture of 2-[(7-iodo-2-naphthalenyl)oxy]-2-methoxyacetic acid (i.e. the product of Step E) (320 mg, 0.89 mmol) and 2-chloro-1-methylpyridium iodide (256 mg, 1.0 mmol) in dichloromethane (6 mL) at 0° C. was added N,N-diisopropylethylamine (0.61 mL, 3.5 mmole) with stirring and ice-bath cooling. The reaction mixture was then stirred at ambient temperature for 15 min and then a solution of 1-methoxy-2-methyl-2-propanamine (114 mg, 1.1 mmol) in dichloromethane (1 mL) was added. The mixture was stirred at ambient temperature for 18 hours and then diluted with 10 mL of dichloromethane. The organic phase was washed with water twice (15 mL each), dried over magnesium sulfate and concentrated under reduced pressure. The residue was purified by column chromatography (over silica gel with ethyl acetate and hexanes in volume ratios from 10 to 53% as eluents) to give the title compound, a compound of the present invention, as an oil (320 mg).

¹H NMR (CDCl₃) δ 1.38 (s, 3H), 1.4 (s, 3H), 3.35-3.43 (m, 5H), 3.5 (s, 3H), 5.38 (s, 1H), 6.8 (s, 1H), 7.29 (d, 1H), 7.37 (s, 1H), 7.5 (d, 1H), 7.6 (d, 1H), 7.72 (d, 1H), 8.15 (s, 1H).

### Example 19

### Preparation of N-(1-cyano-1-methylethyl)-2-[(7-ethynyl-2-naphthalenyl)oxy]butanamide (Compound 110)

To a mixture of 2-[(7-ethynyl-2-naphthalenyl)oxy]butanoic acid (i.e. the product of Example 12, Step E) (508 mg, 2 mmol) and 2-chloro-1-methylpyridium iodide (562 mg, 2.2 mmol) in dichloromethane (12 mL) at 0° C. under nitrogen atomosphere was added N,N-diisopropylethylamine (1.4 mL, 8 mmole). The reaction mixture was stirred at ambient temperature for 15 min and then a solution of 2-aminoisobutylnitrile (217 mg, 2.7 mmol) in dichloromethane (1 mL) was added. The mixture was stirred at room temperature overnight and then diluted with 30 mL of dichloromethane. The organic phase was washed with water twice (35 mL each), dried over magnesium sulfate and concentrated under reduced pressure. The residue was purified by column chromatography (over silica gel with ethyl acetate and hexanes in volume ratios from 10 to 53% as eluents) to give the title compound, a compound of the present invention, as an oil (340 mg).

¹H NMR (CDCl₃) δ 1.06 (t, 3H), 1.66 (s, 3H), 1.69 (s, 3H), 2.05 (m, 2H), 3.16 (s, 1H), 4.7 (t, 1H), 6.52 (s, 1H), 7.11 (s, 1H), 7.2 (d, 1H), 7.43 (d, 1H), 7.7-7.8 (m, 2H), 7.9 (s, 1H).

### Example 20

### Preparation of N-(1,1-dimethyl-2-butyn-1-yl)-2-[(7-ethynyl-2-naphthalenyl)oxy]butanamide (Compound 111)

To a mixture of 2-[(7-ethynyl-2-naphthalenyl)oxy]butanoic acid (i.e. the product of Example 12, Step E) (380 mg, 1.5 mmol) and 2-chloro-1-methylpyridium iodide (421 mg, 1.65 mmol) in dichloromethane (9 mL) at 0° C. under nitrogen atomosphere was added N,N-diisopropylethylamine (1.3 mL, 7.5 mmole). The reaction mixture was stirred at ambient temperature for 15 min and then intermediate 2-methyl-3-pentyn-2-amine hydrochloride (265 mg, 2 mmole) was added. The mixture was stirred at ambient temperature overnight. The reaction mixture was diluted with 30 mL of dichloromethane and washed with water twice (35 mL each). The organic phase was dried over magnesium sulfate and concentrated under reduced pressure. The residue was purified by column chromatography (over silica gel with ethyl acetate and hexanes in volume ratios from 10 to 53% as eluents) to give the title compound, a compound of the present invention, as an oil (135 mg).

¹H NMR (CDCl₃) δ 1.06 (t, 3H), 1.57 (s, 3H), 1.59 (s, 3H), 1.77 (s, 3H), 2.05 (m, 2H), 3.15 (s, 1H), 4.56 (t, 1H), 6.43 (s, 1H), 7.11 (s, 1H), 7.2 (d, 1H), 7.43 (d, 1H), 7.7-7.8 (m, 2H), 7.9 (s, 1H).

### Example 21

### Preparation of N-(1-cyano-1-methylethyl)-2-[(7-ethoxy-2-naphthalenyl)oxy]butanamide (Compound 75)

### Step A: Preparation of methyl 2-[(7-hydroxy-2-naphthalenyl)oxy]butanoate

To a solution of naphthalene-2,7-diol (5.0 g, 31 mmol) in acetone (200 mL) was added cesium carbonate (10.1 g, 31 mmol) at room temperature under a nitrogen atmosphere with stirring. After the addition, the mixture was stirred at room temperature for 3 min and then methyl-2-bromobutyrate (5.6 g, 31 mmol) was added. The mixture was stirred at room temperature overnight and then filtered to remove solids. The filtrate was concentrated under reduced pressure and the residue was purified by column chromatography (over silica gel with ethyl acetate and hexanes in volume ratios from 10 to 80% as eluents) to give the title compound (3.9 g) as a solid.

¹H NMR (CDCl₃) δ 7.62-7.70 (m, 2H), 6.85-7.05 (m, 4H), 5.85 (s, 1H), 4.68-4.78 (m, 1H), 3.75 (s, 3H), 2.00-2.10 (m, 2H), 1.08-1.15 (t, 3H).

### Step B: Preparation of methyl 2-[(7-ethoxy-2-naphthalenyl)oxy]butanoate

A mixture of methyl 2-[(7-hydroxy-2-naphthalenyl)oxy]butanoate (i.e. the product of Step A) (0.52 g, 2 mmol), iodoethane (0.2 mL, 2.4 mmol), and potassium carbonate (0.28 g, 2 mmol) in acetone (20 mL) under a nitrogen atmosphere was stirred at room temperature overnight. The solids were removed by filtration. The filtrate was concentrated under reduced pressure and the residue was purified by column chromatography (over silica gel with ethyl acetate and hexanes in volume ratios from 10 to 60% as eluents) to give the title compound (0.4 g) as a solid.

¹H NMR (CDCl₃) δ 7.60-7.68 (m, 2H), 6.90-7.05 (m, 4H), 4.66-4.72 (t, 1H), 4.06-4.15 (m, 2H), 3.73 (s, 3H), 2.00-2.10 (m, 2H), 1.40-1.48 (t, 3H), 1.08-1.15 (t, 3H).

### Step C: Preparation of 2-[(7-ethoxy-2-naphthalenyl)oxy]butanoic acid

To a solution of methyl 2-[(7-ethoxy-2-naphthalenyl)oxy]butanoate (i.e. the product of Step B) (0.4 g, 1.38 mmol) in tetrahydrofuran (10 mL) was added a sodium hydroxide solution (0.23 mL of 50% NaOH sol. in 10 mL H₂O) at 0° C. with stirring. The mixture was stirred at room temperature for 3 hours. The reaction mixture was acidified with concentrated hydrochloric acid to a pH of 3 and then extracted with ethyl acetate three times. The organic phases were combined and washed with saturated aqueous NaCl solution. After drying (MgSO₄) and concentrating under reduced pressure, the resultant crude residue was purified by column chromatography (over silica gel with ethyl acetate and hexanes in volume ratios from 10 to 90% as eluents) to give the title compound as a solid (0.36 g).

¹H NMR (CDCl₃) δ 7.60-7.68 (m, 2H), 6.98-7.05 (m, 4H), 4.72-4.78 (t, 1H), 4.10-4.15 (m, 2H), 2.04-2.12 (m, 2H), 1.40-1.48 (t, 3H), 1.08-1.15 (t, 3H).

### Step D: Preparation of N-(1-cyano-1-methylethyl)-2-[(7-ethoxy-2-naphthalenyl)oxy]-butanamide

To a mixture of 2-[(7-ethoxy-2-naphthalenyl)oxy]butanoic acid (i.e. the product of Step C) (120 mg, 0.44 mmol) and 2-chloro-1-methylpyridinium iodide (130 mg, 0.51 mmol) in dichloromethane (2 mL) at 0° C. was added N,N-diisopropylethylamine (0.3 mL, 1.7 mmol). The reaction mixture was stirred at ambient temperature for 10 min and then a solution of 2-amino-2-methyl-propionitrile (43 mg, 0.51 mmol) in dichloromethane (1 mL) was added. The mixture was stirred at room temperature for 18 hours. The reaction mixture was diluted with 10 mL of dichloromethane, and then washed with H₂O (2×15 mL). The organic phase was dried (MgSO₄) and concentrated under reduced pressure. The resultant residue was purified by column chromatography (over silica gel with ethyl acetate and hexanes in volume ratios from 10 to 90% as eluents) to give the title compound, a compound of the present invention, as a solid (70 mg).

¹H NMR (CDCl₃) δ 7.61-7.68 (m, 2H), 6.95-7.05 (m, 4H), 6.48 (br, 1H), 4.62-4.68 (m, 1H), 4.08-4.15 (m, 2H), 1.95-2.10 (m, 2H), 1.60-1.72 (m, 6H), 1.41-1.48 (t, 3H), 1.02-1.10 (t, 3H).

By the procedures described herein together with methods known in the art, the following compounds of Tables 1A to 3 can be prepared. The following abbreviations are used in the Tables which follow: n means normal, c means cyclo, Pr means propyl and CN means cyano.

TABLE 1A

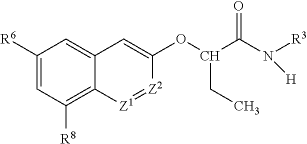

| R³ | R⁶ | R³ | R⁶ |
|---|---|---|---|
| C(CH₃)₂CH₂OCH₃ | Br | C(CH₃)₂CN | OCH₃ |
| C(CH₃)₃ | Br | C(CH₃)₂CH═NNHCHO | OCH₃ |
| N(CH₃)₂ | Br | C(CH₃)₂CH═NN(CH₃)₂ | OCH₃ |
| C(CH₃)₂CH₂OP(O)(CH₃)₂ | Br | C(CH₃)₂CH₂SCH₃ | OCH₃ |
| C(CH₃)₂CH₂NHCHO | Br | C(CH₃)₂CH₂OCH₃ | CH═CH₂ |
| C(CH₃)₂CH₂NHC(O)OC(CH₃)₃ | Br | C(CH₃)₃ | CH═CH₂ |
| 1-methyl-1-cyclopropyl | Br | N(CH₃)₂ | CH═CH₂ |
| C(CH₃)₂CH₂NH₂ | Br | C(CH₃)₂CH₂OP(O)(CH₃)₂ | CH═CH₂ |
| C(CH₃)₂CH₂N═CH(CH₃)₂ | Br | C(CH₃)₂CH₂NHCHO | CH═CH₂ |
| C(CH₃)₂CH₂OH | Br | C(CH₃)₂CH₂NHC(O)OC(CH₃)₃ | CH═CH₂ |
| C(CH₃)₂CH(═O) | Br | 1-methyl-1-cyclopropyl | CH═CH₂ |
| C(CH₃)₂CH₂OCH(═O) | Br | C(CH₃)₂CH₂NH₂ | CH═CH₂ |
| 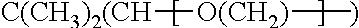 | Br | C(CH₃)₂CH₂N═CH(CH₃)₂ | CH═CH₂ |
| C(CH₃)₂CH₂OCH₂OCH₃ | Br | C(CH₃)₂CH₂OH | CH═CH₂ |
| C(CH₃)₂CH₂F | Br | C(CH₃)₂CH(═O) | CH═CH₂ |
| C(CH₃)₂CF₃ | Br | C(CH₃)₂CH₂OCH(═O) | CH═CH₂ |
| C(CH₃)₂CH₂SC(═O)CH₃ | Br | 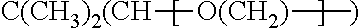 | CH═CH₂ |
| C(CH₃)₂C≡CH | Br | C(CH₃)₂CH₂OCH₂OCH₃ | CH═CH₂ |
| C(CH₃)₂CN | Br | C(CH₃)₂CH₂F | CH═CH₂ |
| C(CH₃)₂CH═NNHCHO | Br | C(CH₃)₂CF₃ | CH═CH₂ |
| C(CH₃)₂CH═NN(CH₃)₂ | Br | C(CH₃)₂CH₂SC(═O)CH₃ | CH═CH₂ |
| C(CH₃)₂CH₂SCH₃ | Br | C(CH₃)₂C≡CH | CH═CH₂ |
| C(CH₃)₂CH₂OCH₃ | I | C(CH₃)₂CN | CH═CH₂ |
| C(CH₃)₃ | I | C(CH₃)₂CH═NNHCHO | CH═CH₂ |
| N(CH₃)₂ | I | C(CH₃)₂CH═NN(CH₃)₂ | CH═CH₂ |
| C(CH₃)₂CH₂OP(O)(CH₃)₂ | I | C(CH₃)₂CH₂SCH₃ | CH═CH₂ |
| C(CH₃)₂CH₂NHCHO | I | C(CH₃)₂CH₂OCH₃ | SCH₃ |
| C(CH₃)₂CH₂NHC(O)OC(CH₃)₃ | I | C(CH₃)₃ | SCH₃ |
| 1-methyl-1-cyclopropyl | I | N(CH₃)₂ | SCH₃ |
| C(CH₃)₂CH₂NH₂ | I | C(CH₃)₂CH₂OP(O)(CH₃)₂ | SCH₃ |
| C(CH₃)₂CH₂N═CH(CH₃)₂ | I | C(CH₃)₂CH₂NHCHO | SCH₃ |
| C(CH₃)₂CH₂OH | I | C(CH₃)₂CH₂NHC(O)OC(CH₃)₃ | SCH₃ |
| C(CH₃)₂CH(═O) | I | 1-methyl-1-cyclopropyl | SCH₃ |
| C(CH₃)₂CH₂OCH(═O) | I | C(CH₃)₂CH₂NH₂ | SCH₃ |
| 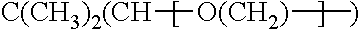 | I | C(CH₃)₂CH₂N═CH(CH₃)₂ | SCH₃ |
| C(CH₃)₂CH₂OCH₂OCH₃ | I | C(CH₃)₂CH₂OH | SCH₃ |
| C(CH₃)₂CH₂F | I | C(CH₃)₂CH(═O) | SCH₃ |
| C(CH₃)₂CF₃ | I | C(CH₃)₂CH₂OCH(═O) | SCH₃ |
| C(CH₃)₂CH₂SC(═O)CH₃ | I | 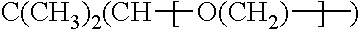 | SCH₃ |
| C(CH₃)₂C≡CH | I | C(CH₃)₂CH₂OCH₂OCH₃ | SCH₃ |
| C(CH₃)₂CN | I | C(CH₃)₂CH₂F | SCH₃ |
| C(CH₃)₂CH═NNHCHO | I | C(CH₃)₂CF₃ | SCH₃ |
| C(CH₃)₂CH═NN(CH₃)₂ | I | C(CH₃)₂CH₂SC(═O)CH₃ | SCH₃ |
| C(CH₃)₂CH₂SCH₃ | I | C(CH₃)₂C≡CH | SCH₃ |
| C(CH₃)₂CH₂OCH₃ | Cl | C(CH₃)₂CN | SCH₃ |
| C(CH₃)₃ | Cl | C(CH₃)₂CH═NNHCHO | SCH₃ |
| N(CH₃)₂ | Cl | C(CH₃)₂CH═NN(CH₃)₂ | SCH₃ |
| C(CH₃)₂CH₂OP(O)(CH₃)₂ | Cl | C(CH₃)₂CH₂SCH₃ | SCH₃ |
| C(CH₃)₂CH₂NHCHO | Cl | C(CH₃)₂CH₂OCH₃ | N(CH₃)₂ |
| C(CH₃)₂CH₂NHC(O)OC(CH₃)₃ | Cl | C(CH₃)₃ | N(CH₃)₂ |
| 1-methyl-1-cyclopropyl | Cl | C(CH₃)₂CH₂OCH₂OCH₃ | N(CH₃)₂ |
| C(CH₃)₂CH₂NH₂ | Cl | C(CH₃)₂CH₂OCH₃ | C≡CCH₃ |
| C(CH₃)₂CH₂N═CH(CH₃)₂ | Cl | C(CH₃)₃ | C≡CCH₃ |
| C(CH₃)₂CH₂OH | Cl | C(CH₃)₂CH₂OCH₂OCH₃ | C≡CCH₃ |
| C(CH₃)₂CH(═O) | Cl | C(CH₃)₂CH₂OCH₃ | SCH₂CH₃ |
| C(CH₃)₂CH₂OCH(═O) | Cl | C(CH₃)₃ | SCH₂CH₃ |
| 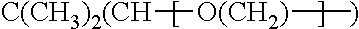 | Cl | C(CH₃)₂CH₂OCH₂OCH₃ | SCH₂CH₃ |
| C(CH₃)₂CH₂OCH₂OCH₃ | Cl | C(CH₃)₂CH₂OCH₃ | OS(═O)₂CF₃ |
| C(CH₃)₂CH₂F | Cl | C(CH₃)₃ | OS(═O)₂CF₃ |
| C(CH₃)₂CF₃ | Cl | C(CH₃)₂CH₂SCH₃ | OS(═O)₂CF₃ |
| C(CH₃)₂CH₂SC(═O)CH₃ | Cl | C(CH₃)₂CH₂OH | OS(═O)₂CF₃ |
| C(CH₃)₂C≡CH | Cl | C(CH₃)₂CH(═O) | OS(═O)₂CF₃ |
| C(CH₃)₂CN | Cl | C(CH₃)₂CH₂OCH(═O) | OS(═O)₂CF₃ |
| C(CH₃)₂CH═NNHCHO | Cl | C(CH₃)₂CH₂OCH₂OCH₃ | OS(═O)₂CF₃ |
| C(CH₃)₂CH═NN(CH₃)₂ | Cl | C(CH₃)₂CH₂F | OS(═O)₂CF₃ |
| C(CH₃)₂CH₂SCH₃ | Cl | C(CH₃)₂CF₃ | OS(═O)₂CF₃ |
| C(CH₃)₂CH₂OCH₃ | ethynyl | C(CH₃)₂C≡CH | OS(═O)₂CF₃ |
| C(CH₃)₃ | ethynyl | C(CH₃)₂CN | OS(═O)₂CF₃ |
| N(CH₃)₂ | ethynyl | C(CH₃)₂CH₂OCH₃ | OCH₂CH₃ |
| C(CH₃)₂CH₂OP(O)(CH₃)₂ | ethynyl | C(CH₃)₃ | OCH₂CH₃ |
| C(CH₃)₂CH₂NHCHO | ethynyl | C(CH₃)₂CH₂OCH₂OCH₃ | OCH₂CH₃ |
| C(CH₃)₂CH₂NHC(O)OC(CH₃)₃ | ethynyl | C(CH₃)₂CH₂OCH₃ | OS(═O)₂CH₃ |
| 1-methyl-1-cyclopropyl | ethynyl | C(CH₃)₃ | OS(═O)₂CH₃ |
| C(CH₃)₂CH₂NH₂ | ethynyl | C(CH₃)₂CH₂OCH₂OCH₃ | OS(═O)₂CH₃ |
| C(CH₃)₂CH₂N═CH(CH₃)₂ | ethynyl | C(CH₃)₂CH₂OCH₃ | OCH(═O) |
| C(CH₃)₂CH₂OH | ethynyl | C(CH₃)₃ | OCH(═O) |
| C(CH₃)₂CH(═O) | ethynyl | C(CH₃)₂CH₂OCH₂OCH₃ | OCH(═O) |
| C(CH₃)₂CH₂OCH(═O) | ethynyl | C(CH₃)₂CH₂OCH₃ | N₃ |
| 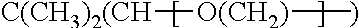 | ethynyl | C(CH₃)₃ | N₃ |
| C(CH₃)₂CH₂OCH₂OCH₃ | ethynyl | C(CH₃)₂CH₂OCH₂OCH₃ | N₃ |
| C(CH₃)₂CH₂F | ethynyl | C(CH₃)₂CH₂OCH₃ | CH₃ |
| C(CH₃)₂CF₃ | ethynyl | C(CH₃)₃ | CH₃ |
| C(CH₃)₂CH₂SC(═O)CH₃ | ethynyl | C(CH₃)₂CH₂OCH₂OCH₃ | CH₃ |
| C(CH₃)₂C≡CH | ethynyl | C(CH₃)₂CH₂OCH₃ | NHCH(═O) |
| C(CH₃)₂CN | ethynyl | C(CH₃)₃ | NHCH(═O) |
| C(CH₃)₂CH═NNHCHO | ethynyl | C(CH₃)₂CH₂OCH₂OCH₃ | NHCH(═O) |
| C(CH₃)₂CH═NN(CH₃)₂ | ethynyl | C(CH₃)₂CH₂OCH₃ | CH₂CH₃ |
| C(CH₃)₂CH₂SCH₃ | ethynyl | C(CH₃)₃ | CH₂CH₃ |
| C(CH₃)₂CH₂OCH₃ | NH₂ | C(CH₃)₂CH₂OCH₂OCH₃ | CH₂CH₃ |
| C(CH₃)₃ | NH₂ | C(CH₃)₂CH₂OCH₃ | OCHF₂ |
| N(CH₃)₂ | NH₂ | C(CH₃)₃ | OCHF₂ |
| C(CH₃)₂CH₂OP(O)(CH₃)₂ | NH₂ | C(CH₃)₂CH₂OCH₂OCH₃ | OCHF₂ |
| C(CH₃)₂CH₂NHCHO | NH₂ | C(CH₃)₂CH₂OCH₃ | CHF₂ |
| C(CH₃)₂CH₂NHC(O)OC(CH₃)₃ | NH₂ | C(CH₃)₃ | CHF₂ |
| 1-methyl-1-cyclopropyl | NH₂ | C(CH₃)₂CH₂OCH₂OCH₃ | CHF₂ |
| C(CH₃)₂CH₂NH₂ | NH₂ | C(CH₃)₂CH₂OCH₃ | CF₃ |
| C(CH₃)₂CH₂N═CH(CH₃)₂ | NH₂ | C(CH₃)₃ | CF₃ |
| C(CH₃)₂CH₂OH | NH₂ | C(CH₃)₂CH₂OCH₂OCH₃ | CF₃ |
| C(CH₃)₂CH(═O) | NH₂ | C(CH₃)₂CH₂OCH₃ | OS(═O)₂CH₃ |
| C(CH₃)₂CH₂OCH(═O) | NH₂ | C(CH₃)₃ | OS(═O)₂CH₃ |
| 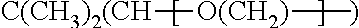 | NH₂ | C(CH₃)₂CH₂OCH₂OCH₃ | OS(═O)₂CH₃ |
| C(CH₃)₂CH₂OCH₂OCH₃ | NH₂ | C(CH₃)₂CH₂OCH₃ | CN |
| C(CH₃)₂CH₂F | NH₂ | C(CH₃)₃ | CN |
| C(CH₃)₂CF₃ | NH₂ | C(CH₃)₂CH₂OCH₂OCH₃ | CN |
| C(CH₃)₂CH₂SC(═O)CH₃ | NH₂ | C(CH₃)₂CH₂OCH₃ | F |
| C(CH₃)₂C≡CH | NH₂ | C(CH₃)₃ | F |
| C(CH₃)₂CN | NH₂ | C(CH₃)₂CH₂OCH₂OCH₃ | F |
| C(CH₃)₂CH═NNHCHO | NH₂ | C(CH₃)₂CH₂OCH₃ | OCF₃ |
| C(CH₃)₂CH═NN(CH₃)₂ | NH₂ | C(CH₃)₃ | OCF₃ |
| C(CH₃)₂CH₂SCH₃ | NH₂ | C(CH₃)₂CH₂OCH₂OCH₃ | OCF₃ |
| C(CH₃)₂CH₂OCH₃ | OCH₃ | C(CH₃)₂CH₂OCH₃ | CH₂F |
| C(CH₃)₃ | OCH₃ | C(CH₃)₃ | CH₂F |
| N(CH₃)₂ | OCH₃ | C(CH₃)₂CH₂OCH₂OCH₃ | CH₂F |
| C(CH₃)₂CH₂OP(O)(CH₃)₂ | OCH₃ | C(CH₃)₂CH₂OCH₃ | c-propyl |
| C(CH₃)₂CH₂NHCHO | OCH₃ | C(CH₃)₃ | c-propyl |
| C(CH₃)₂CH₂NHC(O)OC(CH₃)₃ | OCH₃ | C(CH₃)₂CH₂OCH₂OCH₃ | c-propyl |
| 1-methyl-1-cyclopropyl | OCH₃ | C(CH₃)₂CH₂OCH₃ | CH(═O) |
| C(CH₃)₂CH₂NH₂ | OCH₃ | C(CH₃)₃ | CH(═O) |
| C(CH₃)₂CH₂N═CH(CH₃)₂ | OCH₃ | C(CH₃)₂CH₂OCH₂OCH₃ | CH(═O) |
| C(CH₃)₂CH₂OH | OCH₃ | C(CH₃)₂CH₂OCH₃ | NHCH₃ |
| C(CH₃)₂CH(═O) | OCH₃ | C(CH₃)₃ | NHCH₃ |
| C(CH₃)₂CH₂OCH(═O) | OCH₃ | C(CH₃)₂CH₂OCH₂OCH₃ | NHCH₃ |
| 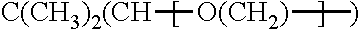 | OCH₃ | C(CH₃)₂CH₂OCH₃ | n-propyl |
| C(CH₃)₂CH₂OCH₂OCH₃ | OCH₃ | C(CH₃)₃ | n-propyl |
| C(CH₃)₂CH₂F | OCH₃ | C(CH₃)₂CH₂OCH₂OCH₃ | n-propyl |
| C(CH₃)₂CF₃ | OCH₃ | C(CH₃)₂CH₂OCH₃ | CHFCH₃ |
| C(CH₃)₂CH₂SC(═O)CH₃ | OCH₃ | C(CH₃)₃ | CHFCH₃ |
| C(CH₃)₂C≡CH | OCH₃ | C(CH₃)₂CH₂OCH₂OCH₃ | CHFCH₃ |

R⁸ is H;
Z¹ is CH;
Z² is CH

### Table 1B

Table 1B is constructed the same as Table 1A, except that Z¹ is N.

### Table 1C

Table 1C is constructed the same as Table 1A, except that Z² is N.

### TABLE 1D

Table 1D is constructed the same as Table 1A, except that Z¹ is N and R⁸ is Cl.

TABLE 2A

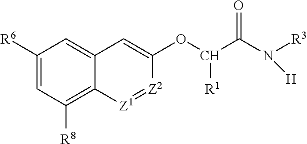

| R³ | R⁶ | R³ | R⁶ |
|---|---|---|---|
| C(CH₃)₂CH₂OCH₃ | Br | C(CH₃)₂CH₂F | OCH₃ |
| C(CH₃)₃ | Br | C(CH₃)₂CF₃ | OCH₃ |
| C(CH₃)₂CH₂SCH₃ | Br | C(CH₃)₂C≡CH | OCH₃ |
| C(CH₃)₂CH₂OH | Br | C(CH₃)₂CN | OCH₃ |
| C(CH₃)₂CH₂OCH₂OCH₃ | Br | C(CH₃)₂CH₂OCH₃ | OS(═O)₂CF₃ |
| C(CH₃)₂CH₂F | Br | C(CH₃)₃ | OS(═O)₂CF₃ |
| C(CH₃)₂CF₃ | Br | C(CH₃)₂CH₂SCH₃ | OS(═O)₂CF₃ |
| C(CH₃)₂C≡CH | Br | C(CH₃)₂CH₂OH | OS(═O)₂CF₃ |
| C(CH₃)₂CN | Br | C(CH₃)₂CH₂OCH₂OCH₃ | OS(═O)₂CF₃ |
| C(CH₃)₂CH₂OCH₃ | I | C(CH₃)₂CH₂F | OS(═O)₂CF₃ |
| C(CH₃)₃ | I | C(CH₃)₂CF₃ | OS(═O)₂CF₃ |
| C(CH₃)₂CH₂SCH₃ | I | C(CH₃)₂C≡CH | OS(═O)₂CF₃ |
| C(CH₃)₂CH₂OH | I | C(CH₃)₂CN | OS(═O)₂CF₃ |
| C(CH₃)₂CH₂OCH₂OCH₃ | I | C(CH₃)₂CH₂OCH₃ | SCH₃ |
| C(CH₃)₂CH₂F | I | C(CH₃)₃ | SCH₃ |
| C(CH₃)₂CF₃ | I | C(CH₃)₂CH₂SCH₃ | SCH₃ |
| C(CH₃)₂C≡CH | I | C(CH₃)₂CH₂OH | SCH₃ |
| C(CH₃)₂CN | I | C(CH₃)₂CH₂OCH₂OCH₃ | SCH₃ |
| C(CH₃)₂CH₂OCH₃ | Cl | C(CH₃)₂CH₂F | SCH₃ |
| C(CH₃)₃ | Cl | C(CH₃)₂CF₃ | SCH₃ |
| C(CH₃)₂CH₂SCH₃ | Cl | C(CH₃)₂C≡CH | SCH₃ |
| C(CH₃)₂CH₂OH | Cl | C(CH₃)₂CN | SCH₃ |
| C(CH₃)₂CH₂OCH₂OCH₃ | Cl | C(CH₃)₂CH₂OCH₃ | NH₂ |
| C(CH₃)₂CH₂F | Cl | C(CH₃)₃ | NH₂ |
| C(CH₃)₂CF₃ | Cl | C(CH₃)₂CH₂SCH₃ | NH₂ |
| C(CH₃)₂C≡CH | Cl | C(CH₃)₂CH₂OH | NH₂ |
| C(CH₃)₂CN | Cl | C(CH₃)₂CH₂OCH₂OCH₃ | NH₂ |
| C(CH₃)₂CH₂OCH₃ | C≡CH | C(CH₃)₂CH₂F | NH₂ |
| C(CH₃)₃ | C≡CH | C(CH₃)₂CF₃ | NH₂ |
| C(CH₃)₂CH₂SCH₃ | C≡CH | C(CH₃)₂C≡CH | NH₂ |
| C(CH₃)₂CH₂OH | C≡CH | C(CH₃)₂CN | NH₂ |
| C(CH₃)₂CH₂OCH₂OCH₃ | C≡CH | C(CH₃)₂CH₂OCH₃ | CH═CH₂ |
| C(CH₃)₂CH₂F | C≡CH | C(CH₃)₃ | CH═CH₂ |
| C(CH₃)₂CF₃ | C≡CH | C(CH₃)₂CH₂SCH₃ | CH═CH₂ |
| C(CH₃)₂C≡CH | C≡CH | C(CH₃)₂CH₂OH | CH═CH₂ |
| C(CH₃)₂CN | C≡CH | C(CH₃)₂CH₂OCH₂OCH₃ | CH═CH₂ |
| C(CH₃)₂CH₂OCH₃ | OCH₃ | C(CH₃)₂CH₂F | CH═CH₂ |
| C(CH₃)₃ | OCH₃ | C(CH₃)₂CF₃ | CH═CH₂ |
| C(CH₃)₂CH₂SCH₃ | OCH₃ | C(CH₃)₂C≡CH | CH═CH₂ |
| C(CH₃)₂CH₂OH | OCH₃ | C(CH₃)₂CN | CH═CH₂ |
| C(CH₃)₂CH₂OCH₂OCH₃ | OCH₃ | | |

R⁸ is H;
Z¹ is CH;
Z² is CH;
R¹ is CH₃

### Table 2B

Table 2B is constructed the same as Table 2A, except that R¹ is OCH₃.

### Table 2C

Table 2C is constructed the same as Table 2A, except that R¹ is n-Pr.

### Table 2D

Table 2D is constructed the same as Table 2A, except that Z¹ is N.

### Table 2E

Table 2E is constructed the same as Table 2A, except that R¹ is OCH₃ and Z¹ is N.

### Table 2F

Table 2F is constructed the same as Table 2A, except that R¹ is n-Pr and Z¹ is N.

### Table 2G

Table 2G is constructed the same as Table 2A, except that Z² is N.

### Table 2H

Table 2H is constructed the same as Table 2A, except that R¹ is OCH₃ and Z² is N.

### Table 2I

Table 2I is constructed the same as Table 2A, except that R¹ is n-Pr and Z² is N.

### Table 2J

Table 2J is constructed the same as Table 2A, except that Z¹ is N and R⁸ is Cl.

### Table 2K

Table 2K is constructed the same as Table 2A, except that R¹ is OCH₃, Z¹ is N and R⁸ is Cl.

### Table 2L

Table 2L is constructed the same as Table 2A, except that R¹ is n-Pr, Z¹ is N and R⁸ is Cl.

TABLE 3

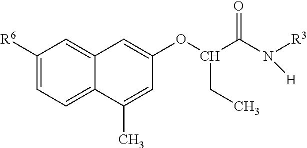

| R³ | R⁶ | R³ | R⁶ |
|---|---|---|---|
| C(CH₃)₂CH₂OCH₃ | Br | C(CH₃)₂CH₂OCH₃ | C≡CH |
| C(CH₃)₃ | Br | C(CH₃)₃ | C≡CH |
| C(CH₃)₂CH₂SCH₃ | Br | C(CH₃)₂CH₂SCH₃ | C≡CH |
| C(CH₃)₂CH₂OH | Br | C(CH₃)₂CH₂OH | C≡CH |
| C(CH₃)₂CH₂OCH₂OCH₃ | Br | C(CH₃)₂CH₂OCH₂OCH₃ | C≡CH |
| C(CH₃)₂CH₂F | Br | C(CH₃)₂CH₂F | C≡CH |
| C(CH₃)₂CF₃ | Br | C(CH₃)₂CF₃ | C≡CH |
| C(CH₃)₂C≡CH | Br | C(CH₃)₂C≡CH | C≡CH |
| C(CH₃)₂CN | Br | C(CH₃)₂CN | C≡CH |
| C(CH₃)₂CH₂OCH₃ | OCH₃ | C(CH₃)₂CH₂OCH₃ | SCH₃ |
| C(CH₃)₃ | OCH₃ | C(CH₃)₃ | SCH₃ |
| C(CH₃)₂CH₂SCH₃ | OCH₃ | C(CH₃)₂CH₂SCH₃ | SCH₃ |
| C(CH₃)₂CH₂OH | OCH₃ | C(CH₃)₂CH₂OH | SCH₃ |
| C(CH₃)₂CH₂OCH₂OCH₃ | OCH₃ | C(CH₃)₂CH₂OCH₂OCH₃ | SCH₃ |
| C(CH₃)₂CH₂F | OCH₃ | C(CH₃)₂CH₂F | SCH₃ |
| C(CH₃)₂CF₃ | OCH₃ | C(CH₃)₂CF₃ | SCH₃ |
| C(CH₃)₂C≡CH | OCH₃ | C(CH₃)₂C≡CH | SCH₃ |
| C(CH₃)₂CN | OCH₃ | C(CH₃)₂CN | SCH₃ |
| C(CH₃)₂CH₂OCH₃ | I | C(CH₃)₂CH₂OCH₃ | NH₂ |
| C(CH₃)₃ | I | C(CH₃)₃ | NH₂ |
| C(CH₃)₂CH₂SCH₃ | I | C(CH₃)₂CH₂SCH₃ | NH₂ |
| C(CH₃)₂CH₂OH | I | C(CH₃)₂CH₂OH | NH₂ |
| C(CH₃)₂CH₂OCH₂OCH₃ | I | C(CH₃)₂CH₂OCH₂OCH₃ | NH₂ |
| C(CH₃)₂CH₂F | I | C(CH₃)₂CH₂F | NH₂ |
| C(CH₃)₂CF₃ | I | C(CH₃)₂CF₃ | NH₂ |
| C(CH₃)₂C≡CH | I | C(CH₃)₂C≡CH | NH₂ |
| C(CH₃)₂CN | I | C(CH₃)₂CN | NH₂ |
| C(CH₃)₂CH₂OCH₃ | Cl | C(CH₃)₂CH₂OCH₃ | CH═CH₂ |
| C(CH₃)₃ | Cl | C(CH₃)₃ | CH═CH₂ |
| C(CH₃)₂CH₂SCH₃ | Cl | C(CH₃)₂CH₂SCH₃ | CH═CH₂ |
| C(CH₃)₂CH₂OH | Cl | C(CH₃)₂CH₂OH | CH═CH₂ |
| C(CH₃)₂CH₂OCH₂OCH₃ | Cl | C(CH₃)₂CH₂OCH₂OCH₃ | CH═CH₂ |
| C(CH₃)₂CH₂F | Cl | C(CH₃)₂CH₂F | CH═CH₂ |
| C(CH₃)₂CF₃ | Cl | C(CH₃)₂CF₃ | CH═CH₂ |
| C(CH₃)₂C≡CH | Cl | C(CH₃)₂C≡CH | CH═CH₂ |
| C(CH₃)₂CN | Cl | C(CH₃)₂CN | CH═CH₂ |

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-75 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual international and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in index Tables A-C. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

High Strength Concentrate

| | |
|---|---|
| Compound 61 | 98.5% |
| silica aerogel | 0.5% |
| synthetic amorphous fine silica | 1.0% |

### Example B

Wettable Powder

| | |
|---|---|
| Compound 51 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 3 | 10.0% |
| attapulgite granules (low volatile matter, 0.71/0.30 mm; | 90.0% |
| U.S.S. No, 25---50 sieves) | |

### Example D

Extruded Pellet

| | |
|---|---|
| Compound 7 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 8 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 15 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 20 | 20.00% |
| polyvinylpyrrolidone-vinyi acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora inqfestans, Phytophthora megasperma, Phytophthora parasitica, Phytophthora cinnamomi and Phytophthora capsici, Pythiumn diseases such as Pythium aphanidermatum, and diseases in the Peronosporaceae family such as Plasmopara viticola, Peronospora spp. (including Peronospora tabacina and Peronospora parasitica), Pseudioperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondita, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Accordingly, this aspect of the present invention can also be described as a method for protecting a plant or plant seed from diseases caused by fungal pathogens comprising applying a fungicidally effective amount of a compound of Formula 1, an N-oxide, or salt thereof to the plant (or portion thereof) or plant seed (directly or through the environment (e.g., growing medium) of the plant or plant seed).

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 includes at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸-Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, N-[2-(1S,2R)-[1,1′-bicyclopropyl]-2-ylphenyl]-3-(difluoromethyl)-1-methyl-1H-pyrazole-4-carboxamide and penflufen (N-[2-(1,3-dimethyl-butyl)phenyl]-5-fluoro-1,3-dimethyl-1H-pyrazole-4-carboxamide). The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071), picoxystrobin and pyraoxystrobin (SYP-3343). The methoxycarbamates include pyraclostrobin and pyrametostrobin (SYP-4155). The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen and tebufloquin are examples of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly. Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb, valifenalate and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-S-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code MS) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7), (46.4) “benzophenone fungicides” (Fungicide Resistance Action Committee (FRAC) code U8) and (46.5) “triazolopyrimidine fungicides”. The thiazole carboxamides include ethaboxam. The phenyl-acetamides include cyflufenamid and N-[[(cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrrolnitrin, quinomethionate, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, 2-[[2-fluoro-5-(trifluoromethyl)phenyl]thio]-2-[3-(2-methoxyphenyl)-2-thiazolidinylidene]acetonitrile, 3-[5-(4-chlorophenyl)-2,3-dimethyl-3-isoxazolidinyl]pyridine, 4-fluoro-phenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl)[1,2,4]triazolo[1,5-a]pyrimidine, N-(4-chloro-2-nitrophenyl)-1-ethyl-4-methylbenzenesulfonamide, N-[[(cyclopropylmethoxy)-amino][6-(difluoromethoxy)-2,3-difluorophenyl]methylene]benzeneacetamide, N-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one. The triazolopyrimidines include ametoctradin. (46.6) Other fungicides whose mode of action may be unknown include pentyl N-[4-[[[[(1-methyl-1H-tetrazol-5-yl)phenylmethylene]amino]oxy]methyl]-2-thiazolyl]carbamate, pentyl N-[6-[[[[(1-methyl-1H-tetrazol-5-yl)phenylmethylene]amino]oxy]methyl]-2-pyridinyl]carbamate and Hambra®.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acrinathrin, amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, milbemycin oxime, monocrotophos, methoxyfenozide, nicotine, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) proquinazid (6-iodo-3-propyl-2-propyloxy-4(3H)-quinazolinone); (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14 DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

The following Tests demonstrate the control efficacy of compounds of this invention on specific pathogens. The pathogen control protection afforded by the compounds is not limited, however, to these species. See Index Tables A-E for compound descriptions. The following abbreviations are used in the Index Tables which follow: Me is methyl and CN is cyano.

The compounds of this invention prepared by the methods described herein are shown in Index Tables A-E. For mass spectral data (MS⁺(M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺) or electrospray ionization (ESI). The alternate molecular ion peaks (e.g., M+2 or M+4) that occur with compounds containing multiple halogens are not reported.

INDEX TABLE A

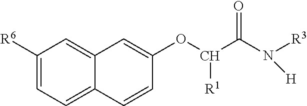

| Cmpd. | R⁶ | R¹ | R³ | MS (M + 1) |
|---|---|---|---|---|
| 1 | Br | CH₂CH₃ | C(CH₃)₂CN | 376 |
| 3 | OCH₃ | CH₂CH₃ | C(CH₃)₃ | 316 |
| 4 | OCH₃ | CH₂CH₃ | C(CH₃)₂C≡CH | 326 |
| 5 | OCHF₂ | CH₂CH₃ | C(CH₃)₃ | 352 |
| 6 | OCHF₂ | CH₂CH₃ | C(CH₃)₂C≡CH | 362 |
| 7 | Br | CH₂CH₃ | C(CH₃)₂CH₂OCH₃ | 394 |
| 8 | Br | CH₂CH₃ | C(CH₃)₃ | 364 |
| 9 | Br | CH₂CH₃ | N(CH₃)₂ | 351 |
| 10 | Br | CH₂CH₃ | C(CH₃)₂CH₂OP(O)CH₃₎₂ | 456 |
| 11 | Br | CH₂CH₃ | C(CH₃)₂CH₂NHCHO | 407 |
| 12 | Br | CH₂CH₃ | C(CH₃)₂CH₂HNC(O)OC(CH₃)₃ | * |
| 13 | Br | CH₂CH₃ | 1-methyl-1-cyclopropyl | 362 |
| 14 | Br | CH₂CH₃ | C(CH₃)₂CH₂NH₂ | 379 |
| 15 | Br | CH₂CH₃ | C(CH₃)₂CH₂NHCHN(CH₃)₂ | 434 |
| 16 | Br | CH₂CH₃ | C(CH₃)₂CH₂OH | 380 |
| 17 | Br | CH₂CH₃ | C(CH₃)₂CHO | 378 |
| 18 | Br | CH₂CH₃ | C(CH₃)₂CH₂OCHO | 408 |
| 19 | Br | CH₂CH₃ | 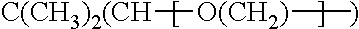 | 392 |
| 20 | Br | CH₂CH₃ | C(CH₃)₂CH₂OCH₂OCH₃ | 424 |
| 21 | CN | CH₂CH₃ | C(CH₃)₂CH₂OCH₃ | 341 |
| 22 | CN | CH₂CH₃ | C(CH₃)₂CH₂OCH₂OCH₃ | 371 |
| 24 | Br | CH₃ | C(CH₃)₂CH₂OCH₃ | 380 |
| 25 | CN | CH₃ | C(CH₃)₂CH₂OCH₃ | 327 |
| 26 | Br | CH₂CH₃ | C(CH₃)₂CH₂F | 382 |
| 27 | Br | CH₂CH₃ | C(CH₃)₂CF₃ | 418 |
| 28 | Br | CH₂CH₃ | C(CH₃)₂CH₂SC(O)CH₃ | 438 |
| 29 | NH₂ | CH₂CH₃ | C(CH₃)₃ | 301 |
| 31 | Br | CH₂CH₃ | C(CH₃)₂C≡CH | 374 |
| 32 | Br | COCH₃ | C(CH₃)₃ | 378 |
| 33 | CN | CH₂CH₃ | C(CH₃)₃ | 311 |
| 68 | OCH₂CH₃ | OCH₃ | C(CH₃)₂CN | 341# |
| 69 | OCH₂CH₃ | OCH₃ | C(CH₃)₂C≡CH | * |
| 70 | OCH₂CH₃ | OCH₃ | C(CH₃)₂CH₂OCH₃ | 362 |
| 71 | OCH₂OCH₃ | CH₂CH₃ | C(CH₃)₃ | 346 |
| 72 | OCH₂OCH₃ | CH₂CH₃ | C(CH₃)₂CN | 355# |
| 73 | OCH₂OCH₃ | CH₂CH₃ | C(CH₃)₂C≡CH | 356 |
| 74 | OCH₂CH₃ | CH₂CH₃ | C(CH₃)₃ | 330 |
| 75 | OCH₂CH₃ | CH₂CH₃ | C(CH₃)₂CN | 341 |
| 76 | OCH₂CH₃ | CH₂CH₃ | C(CH₃)₂C≡CH | 340 |
| 77 | CHF₂ | CH₂CH₃ | C(CH₃)₂CN | 347 |
| 78 | CHF₂ | CH₂CH₃ | C(CH₃)₂C≡CH | 346 |
| 79 | CHF₂ | OCH₃ | C(CH₃)₂CN | 347# |
| 80 | CHF₂ | OCH₃ | C(CH₃)₂C≡CH | 348 |
| 81 | OCH₃ | OCH₃ | C(CH₃)₂CN | 329 |
| 82 | OCH₃ | OCH₃ | C(CH₃)₂C≡CH | 328 |
| 83 | OCH₃ | OCH₃ | C(CH₃)₂CH₂OCH₃ | 348 |
| 84 | OCH₃ | CH₂CH₃ | C(CH₃)₂COOCH₃ | 360 |
| 85 | OCH₃ | CH₂CH₃ | 1-ethynylcyclohexyl | 366 |
| 86 | OCH₃ | CH₂CH₃ | C(CH₂CH₃)₂C≡CH | 354 |
| 87 | OCH₃ | CH₂CH₃ | C(CH₃)₂CN | 327 |
| 88 | OCH₃ | CH₂CH₃ | C(CH₃)₂CH₂OCH₃ | 346 |
| 89 | OH | CH₂CH₃ | C(CH₃)₃ | 302 |
| 90 | Br | OCH₃ | C(CH₃)₂CH₂OCH₃ | 396 |
| 91 | SCH₃ | CH₂CH₃ | C(CH₃)₂CH₂OCH₃ | 362 |
| 92 | I | CH₂CH₃ | C(CH₃)₂CH₂OCH₃ | 442 |
| 93 | Br | OCH₃ | C(CH₃)₂CN | 377 |
| 94 | Br | OCH₃ | C(CH₃)₂C≡CH | 376 |
| 95 | Br | CH₂CH₃ | C(CH₃)₂CN(O)CH₃ | 407 |
| 96 | C≡CSi(CH₃)₃ | CH₂CH₃ | C(CH₃)₂CH₂OCH₃ | 412 |
| 97 | C≡CH | CH₂CH₃ | C(CH₃)₂CH₂OCH₃ | 340 |
| 98 | Br | OCH₃ | C(CH₃)₂CH₂OH | 382 |
| 99 | Br | OCH₃ | C(CH₃)₂CH₂OCH₂OCH₃ | 426 |
| 100 | Br | OCH₃ | C(CH₃)₃ | 366 |
| 101 | Br | OCH₃ | C(CH₃)₂CH₂CH₃ | 380 |
| 102 | Br | CH₂CH₃ | C(CH₃)₂CH₂CH₃ | 378 |
| 103 | I | OCH₃ | C(CH₃)₂CH₂OCH₃ | 444 |
| 104 | I | OCH₃ | C(CH₃)₂CN | 425 |
| 105 | C≡CSi(CH₃)₃ | OCH₃ | C(CH₃)₂CH₂OCH₃ | 414 |
| 106 | C≡CH | OCH₃ | C(CH₃)₂CH₂OCH₃ | 342 |
| 107 | C≡CCH₃ | CH₂CH₃ | C(CH₃)₂CH₂OCH₃ | 354 |
| 108 | C≡CCH₃ | OCH₃ | C(CH₃)₂CH₂OCH₃ | 356 |
| 109 | C≡CH | CH₂CH₃ | C(CH₃)₃ | 310 |
| 110 | C≡CH | CH₂CH₃ | C(CH₃)₂CN | 321 |
| 111 | C≡CH | CH₂CH₃ | C(CH₃)₂C≡CCH₃ | 334 |
| 112 | C≡CH | CH₂CH₃ | C(CH₃)₂CH₂OCH₂OCH₃ | 370 |
| 113 | OCH₃ | OCH₂CH₃ | C(CH₃)₂CH₂OCH₃ | 362 |
| 114 | OCH₃ | OCH₂CH₃ | C(CH₃)₃ | 332 |
| 115 | OCH₃ | OCH₂CH₃ | C(CH₃)₂C≡CH | 342 |
| 116 | OCH₃ | OCH₂CH₃ | C(CH₃)₂CN | 343 |
| 117 | OCH₃ | OCH₂CH₃ | C(CH₃)₂CH₂CH₃ | 346 |
| 118 | Br | OCH₂CH₃ | C(CH₃)₂CH₂OCH₃ | 411 |
| 119 | Br | OCH₂CH₃ | C(CH₃)₃ | 381 |
| 120 | OCH₂CH₃ | OCH₂CH₃ | C(CH₃)₂CH₂OCH₃ | 376 |
| 121 | OCH₂CH₃ | OCH₂CH₃ | C(CH₃)₃ | 346 |
| 122 | OCH₂CH₃ | OCH₂CH₃ | C(CH₃)₂C≡CH | 356 |
| 123 | OCH₂CH₃ | OCH₂CH₃ | C(CH₃)₂CN | 357 |
| 124 | OCH₂CH₂CH₃ | OCH₃ | C(CH₃)₂CH₂OCH₃ | 376 |
| 125 | OCH₂CH₂CH₃ | OCH₃ | C(CH₃)₃ | 346 |
| 126 | OCH₂CH₂CH₃ | OCH₃ | C(CH₃)₂C≡CH | 357 |
| 127 | OCH₂CH₂CH₃ | CH₂CH₃ | C(CH₃)₂CH₂OCH₃ | 374 |
| 128 | OCH₂CH₂CH₃ | CH₂CH₃ | C(CH₃)₃ | 344 |
| 129 | OCH₂CH₂CH₃ | CH₂CH₃ | C(CH₃)₂C≡CH | 354 |
| 130 | OCH₂C≡CH | OCH₃ | C(CH₃)₂CH₂OCH₃ | 372 |
| 131 | OCH₂C≡CH | OCH₃ | C(CH₃)₃ | 342 |
| 132 | OCH₂C≡CH | OCH₃ | C(CH₃)₂C≡CH | 352 |
| 133 | OCH₂C≡CH | CH₂CH₃ | C(CH₃)₂CH₂OCH₃ | 370 |
| 134 | OCH₂C≡CH | CH₂CH₃ | C(CH₃)₃ | 340 |
| 135 | OCH₂C≡CH | CH₂CH₃ | C(CH₃)₂C≡CH | 350 |
| 136 | OCH₂CH₂CH₃ | OCH₃ | C(CH₃)₂CH₂CH₃ | 360 |
| 137 | OCH₂C≡CH | OCH₃ | C(CH₃)₂CH₂CH₃ | 356 |
| 138 | CH₂CH₃ | CH₂CH₃ | C(CH₃)₂CH₂OCH₃ | 344 |
| 139 | CH₂CH₃ | CH₂CH₃ | C(CH₃)₂CH₂OCH₂CH₃ | 358 |
| 140 | CH₂CH₃ | CH₂CH₃ | C(CH₃)₂CN | 314 |
| 141 | CH₂CH₃ | CH₂CH₃ | C(CH₃)₂C≡CH | 324 |
| 142 | CH₂CH₃ | CH₂CH₃ | C(CH₃)₂C≡CCH₃ | 338 |
| 143 | CH₂CH₂CH₃ | CH₂CH₃ | C(CH₃)₂CH₂OCH₃ | 358 |
| 144 | CH₂CH₂CH₃ | CH₂CH₃ | C(CH₃)₂C≡CH | 338 |
| 145 | C≡CH₂ | CH₂CH₃ | C(CH₃)₂CH₂OCH₃ | 342 |
| 159 | CH₃ | CH₂CH₃ | C(CH₃)₂CH₂OCH₃ | 330 |
| 169 | C≡CH | CH₂CH₃ | C(CH₃)₂CH₂OH | 326 |
| 174 | C≡CH | OCH₃ | C(CH₃)₂CN | 323 |
| 175 | C≡CH | OCH₃ | C(CH₃)₂C≡CH | 322 |
| 176 | C≡CH | OCH₃ | C(CH₃)₃ | 312 |

*See Index Table E for ¹H NMR data.
#M-1 from APCI—

INDEX TABLE B

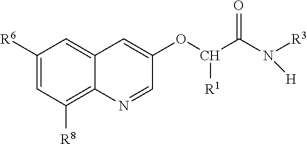

| Cmpd. | R⁶ | R⁸ | R¹ | R³ | MS (M + 1) |
|---|---|---|---|---|---|
| 34 | Br | H | CH₂OCH₃ | C(CH₃)₃ | 382 |
| 35 | Br | H | CH₂OCH₃ | C(CH₃)₂CH₂OCH₃ | 398 |
| 36 | Br | H | CH₂OCH₃ | C(CH₃)₂C≡CH | 392 |
| 37 | Br | H | CH₂CH₃ | C(CH₃)₃ | 366 |
| 38 | Br | H | CH₂CH₃ | C(CH₃)₂C≡CH | 376 |
| 39 | Br | H | CH₂CH₃ | C(CH₃)₂CH₂OH | 382 |
| 40 | Cl | H | CH₂CH₃ | C(CH₃)₃ | 321 |
| 41 | Cl | H | CH₂CH₃ | C(CH₃)₂C≡CH | 331 |
| 42 | Cl | H | CH₂CH₃ | C(CH₃)₂CH₂OH | 337 |
| 43 | Br | H | SCH₃ | C(CH₃)₃ | 384 |
| 44 | Br | H | CH₂CH₃ | C(CH₂)₂CO₂CH₃ | 408 |
| 45 | F | H | CH₂CH₃ | C(CH₃)₃ | 305 |
| 46 | F | H | CH₂CH₃ | C(CH₂)₂CN | 314 |
| 47 | F | H | CH₂CH₃ | C(CH₃)₂C≡CH | 315 |
| 48 | Br | H | CH₂CH₃ | C(CH₃)₂CH₂OCH₃ | 396 |
| 49 | Cl | H | CH₂CH₃ | C(CH₃)₂CH₂OCH₃ | 351 |
| 50 | Cl | H | CH₂CH₃ | C(CH₃)₂CH₂OCH₂CH₃ | 365 |
| 51 | I | H | CH₂CH₃ | C(CH₃)₃ | 413 |
| 52 | I | H | CH₂CH₃ | C(CH₃)₂CH₂OH | 429 |
| 53 | I | H | CH₂CH₃ | C(CH₃)₂CH₂OCH₃ | 443 |
| 54 | F | H | CH₂CH₃ | C(CH₃)₂CH₂OCH₃ | 335 |
| 55 | I | H | CH₂CH₃ | C(CH₃)₂CH₂OCH₂CH₃ | 457 |
| 56 | Cl | Cl | CH₂CH₃ | C(CH₃)₂CH₂OCH₃ | 385 |
| 57 | Cl | Cl | CH₂CH₃ | C(CH₃)₃ | 355 |
| 58 | Br | H | CH₂CH₃ | C(CH₃)₂CH₂OCH₂OCH₃ | 426 |
| 59 | I | H | CH₃ | C(CH₃)₂CH₂OCH₃ | 429 |
| 60 | Br | H | CH₃ | C(CH₃)₂CH₂OCH₃ | 382 |
| 61 | I | H | CH₂CH₃ | C(CH₃)₂CH₂OCH₃ | 443 |
| 62 | I | H | CH₂CH₃ | C(CH₃)₂CH₂OCH₃ | 443** |
| 63 | I | H | OCH₃ | C(CH₃)₂C≡CH | 425 |
| 64 | I | H | OCH₃ | C(CH₂)₂CH₃ | 413 |
| 65 | I | H | OCH₃ | C(CH₃)₂CH₂OCH₃ | 445 |
| 146 | OCH₃ | H | CH₂CH₃ | C(CH₃)₂CH₂OCH₃ | 347 |
| 147 | OCH₃ | H | CH₂CH₃ | C(CH₃)₂C≡CH | 327 |
| 148 | C≡CH | H | CH₂CH₃ | C(CH₃)₂CH₂OCH₃ | 341 |
| 149 | C≡CH | H | CH₂CH₃ | C(CH₃)₃ | 311 |
| 150 | C≡CH | H | CH₂CH₃ | C(CH₃)₂CH₂CH₃ | 325 |
| 151 | C≡CH | H | CH₂CH₃ | C(CH₃)₂C≡CH | 321 |
| 152 | C≡CH | H | CH₂CH₃ | C(CH₃)₂CN | 322 |
| 153 | C≡CH | H | CH₂CH₃ | CH₂C(CH₃)₃ | 325 |
| 154 | Br | H | OCH₃ | C(CH₃)₂CH₂OCH₃ | 398 |
| 155 | Br | H | OCH₃ | C(CH₃)₃ | 368 |
| 156 | C≡CCH₃ | H | OCH₃ | C(CH₃)₃ | 327 |
| 157 | C≡CH | H | CH₂CH₃ | C(CH₃)₂CH₂OCH₂OCH₃ | 355 |
| 158 | C≡CCH₃ | H | OCH₃ | C(CH₃)₂CH₂OCH₃ | 357 |
| 160 | C≡CSi(CH₃)₃ | H | OCH₃ | C(CH₃)₂CH₂OCH₃ | 415 |
| 161 | C≡CSi(CH₃)₃ | H | OCH₃ | C(CH₃)₃ | 385 |
| 162 | C≡CH | H | OCH₃ | C(CH₃)₂CH₂OCH₃ | 343 |
| 163 | C≡CH | H | OCH₃ | C(CH₃)₃ | 313 |

INDEX TABLE C

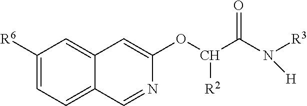

| Cmpd. | R⁶ | R¹ | R³ | MS (M + 1) |
|---|---|---|---|---|
| 66 | F | CH₂CH₃ | C(CH₃)₃ | 305 |
| 67 | Br | CH₂CH₃ | C(CH₃)₃ | 365 |

INDEX TABLE D

| Cmpd. | R³ | MS (M + 1) |
|---|---|---|
| 164 | 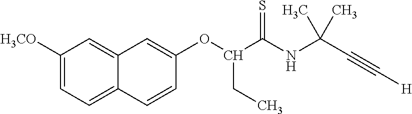 | 342 |
| 165 | 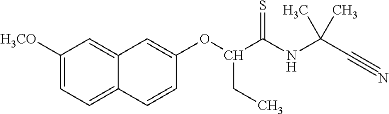 | 344 |
| 166 | 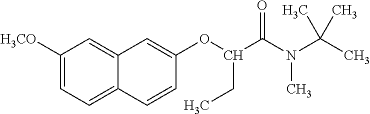 | 330 |
| 167 | 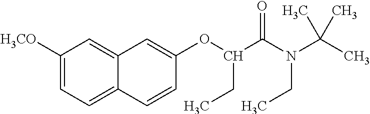 | 344 |
| 168 | 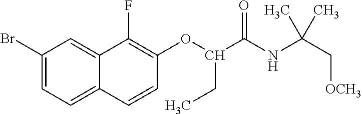 | 412 |
| 170 | 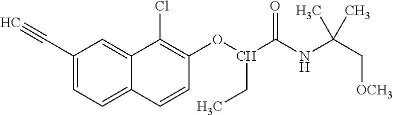 | 374 |
| 171 | 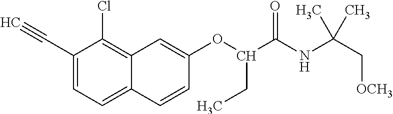 | 374 |
| 172 | 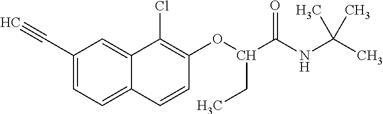 | 344 |
| 173 | 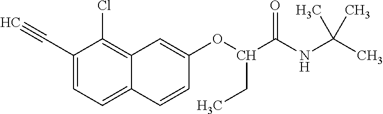 | 344 |

INDEX TABLE E

| Compd. No. | ¹H NMR Data (CDCl₃ solution unless indicated otherwise)^{a} |
|---|---|
| 12 | δ 1.03 (t, 3H), 1.27 (s, 3H), 1.28 (s, 3H), 1.36 (s, 9H), 2.0 (m, 2H), 3.10-3.34 (m, 2H), 4.53 |
| | (t, 11), 5.06 (brs, 1H), 6.95 (brs, 1H), 7.03 (s, 1H), 7.21 (d, 1H), 7,40 (d, 1H), 7.60 (d, 1H), |
| | 7.71 (d, 1H), 7.85 (s, 1H). |
| 69 | δ 7.62-7.72 (m, 2H), 7.38 (m, 1H), 7.1-7.16 (m, 1H), 7.02-7.08 (m, 2H), 6.79 (br, 1H), 5.41 |
| | (s, 1H), 4,08-4,18 (m, 2H) 3.51 (s, 3H), 2.38 (s, 1H), 1.70 (s, 6H), 1.42-1.51 (t, 3H). |

^{a 1}H NMR data are in ppm downfield from tetramethylsilane. Couplings are designated by (s)-singlet,
(d)-doublet, (t)-triplet, (m)-muitiplet and (brs)-broad singlet.

### Biological Examples of the Invention

General protocol for preparing test suspensions for Tests A-C: the test compounds were first dissolved in acetone in an amount equal to 11% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-C. Spraying a 40 ppm test suspension to the point of run-off on the test plants was the equivalent of a rate of 800 g/ha.

### Test A

The test suspension was sprayed to the point of run-off on wheat seedlings. The following day the seedlings were inoculated with a spore dust of Blumeria graminis f. sp. tritici, (the causal agent of wheat powdery mildew) and incubated in a growth chamber at 20° C. for 8 days, after which time disease ratings were visually made.

### Test B

The test suspension was sprayed to the point of run-off on wheat seedlings. The following day the seedlings were inoculated with a spore suspension of Septoria tritici (the causal agent of wheat leaf blotch) and incubated in a saturated atmosphere at 20° C. for 48 h, and then moved to a growth chamber at 20° C. for 19 days, after which time disease ratings were visually made.

### Test C

The test suspension was sprayed to the point of run-off on tomato seedlings. The following day the seedlings were inoculated with a spore suspension of Botrytis cinerea (the causal agent of tomato botrytis) and incubated in saturated atmosphere at 20° C. for 48 h, and then moved to a growth chamber at 27° C. for 3 additional days, after which time disease ratings were visually made.

Results for Tests A-C are given in Table A. In the Table, a rating of 100 indicates 100% disease control and a rating of 0 indicates no disease control (relative to the controls). A dash (-) indicates no test results. All results are from applications of 40 ppm.

TABLE A

| Compound | Test A | Test B | Test C |
|---|---|---|---|
| 1 | 91 | 100 | 100 |
| 3 | 100 | 99 | 99 |
| 4 | 97 | 100 | 100 |
| 5 | 99 | 99 | 100 |
| 6 | 98 | 100 | 100 |
| 7 | 100 | 99 | 99 |
| 8 | 100 | 99 | 99 |
| 9 | 69 | 99 | 99 |
| 10 | 21 | 99 | 98 |
| 11 | 21 | 99 | 100 |
| 12 | 0 | 93 | 0 |
| 13 | 50 | 98 | 99 |
| 14 | 90 | 99 | 99 |
| 15 | 43 | 100 | 100 |
| 16 | 97 | 99 | 100 |
| 17 | 89 | 100 | 99 |
| 18 | 99 | 99 | 100 |
| 19 | 0 | 60 | 98 |
| 20 | 98 | 99 | 98 |
| 21 | 91 | 99 | 99 |
| 22 | 93 | 100 | 100 |
| 24 | 100 | 100 | 98 |
| 25 | 47 | 96 | 99 |
| 26 | 100 | 100 | 99 |
| 27 | 99 | 86 | 98 |
| 28 | 91 | 99 | 99 |
| 29 | 0 | 76 | 95 |
| 31 | 100 | — | 100 |
| 32 | 0 | 0 | 26 |
| 33 | 62 | 99 | 95 |
| 34 | 0 | 92 | 94 |
| 35 | 0 | 0 | 0 |
| 36 | 0 | 89 | 36 |
| 37 | 89 | 100 | 99 |
| 38 | 21 | 100 | 99 |
| 39 | 21 | 0 | 33 |
| 40 | 92 | 100 | 99 |
| 41 | 0 | 96 | 96 |
| 42 | 0 | 0 | 0 |
| 43 | 0 | 99 | 99 |
| 44 | 0 | 0 | 0 |
| 45 | 84 | 42 | 98 |
| 46 | 0 | 0 | 0 |
| 47 | 0 | 20 | 70 |
| 48 | 96 | 99 | 99 |
| 49 | 96 | 99 | 98 |
| 50 | 84 | 98 | 90 |
| 51 | 93 | 100 | 99 |
| 52 | 0 | 45 | 82 |
| 53 | 99 | 100 | 99 |
| 54 | 0 | 71 | 0 |
| 55 | 96 | 99 | 86 |
| 56 | 0 | 99 | 99 |
| 57 | 0 | 99 | 99 |
| 58 | 99 | 100 | 99 |
| 59 | 0 | 95 | 99 |
| 60 | 0 | 74 | 95 |
| 61 | 98 | 100 | 99 |
| 62 | 0 | 0 | 7 |
| 63 | 93 | 99 | 99 |
| 64 | 84 | 99 | 100 |
| 65 | 81 | 99 | 100 |
| 66 | 79 | 0 | 88 |
| 67 | 92 | 77 | 100 |
| 68 | 100 | 100 | 100 |
| 69 | 100 | 100 | 100 |
| 70 | 100 | 100 | 98 |
| 71 | 81 | 99 | 99 |
| 72 | 0 | 99 | 100 |
| 73 | 0 | 100 | 99 |
| 74 | 100 | 100 | 100 |
| 75 | 100 | 100 | 100 |
| 76 | 100 | 100 | 100 |
| 77 | 0 | 99 | 67 |
| 78 | 81 | 88 | 99 |
| 79 | 72 | 98 | 99 |
| 80 | 0 | 93 | 100 |
| 81 | 0 | 99 | 100 |
| 82 | 96 | 99 | 100 |
| 83 | 96 | 99 | 100 |
| 84 | 0 | 84 | 100 |
| 85 | 0 | 84 | 99 |
| 86 | 0 | 19 | 19 |
| 87 | 0 | 100 | 100 |
| 88 | 99 | 100 | 100 |
| 89 | 0 | 63 | 86 |
| 90 | 100 | 100 | 100 |
| 91 | 97 | 100 | 100 |
| 92 | 100 | 100 | 100 |
| 93 | 95 | 100 | 100 |
| 94 | 99 | 100 | 100 |
| 95 | 92 | 100 | 100 |
| 96 | 0 | 100 | 99 |
| 97 | 100 | 100 | 99 |
| 98 | 64 | — | 99 |
| 99 | 96 | — | 100 |
| 100 | 100 | — | 99 |
| 101 | 92 | 100 | 100 |
| 102 | 0 | 96 | 61 |
| 103 | 100 | 100 | 100 |
| 104 | 94 | 100 | 99 |
| 105 | 79 | 100 | 90 |
| 106 | 100 | 100 | 99 |
| 107 | 100 | 100 | 99 |
| 108 | 100 | 100 | 48 |
| 109 | 100 | 100 | 99 |
| 110 | 100 | 100 | 100 |
| 111 | 100 | 100 | 100 |
| 113 | 97 | 100 | 99 |
| 114 | 90 | 99 | 99 |
| 115 | 79 | 99 | 97 |
| 116 | 13 | 99 | 99 |
| 117 | 0 | 93 | 98 |
| 118 | 0 | 97 | 97 |
| 119 | 89 | 93 | 99 |
| 120 | 90 | 99 | 98 |
| 121 | 29 | 100 | 99 |
| 122 | 0 | 100 | 98 |
| 123 | 82 | 100 | 98 |
| 124 | 98 | 100 | 99 |
| 125 | 92 | 100 | 98 |
| 126 | 72 | 99 | 99 |
| 127 | 94 | 99 | 99 |
| 128 | 98 | 100 | 99 |
| 129 | 96 | 99 | 99 |
| 130 | 21 | 98 | 100 |
| 131 | 68 | 100 | 100 |
| 132 | 13 | 99 | 100 |
| 133 | 74 | 99 | 100 |
| 134 | 43 | 100 | 100 |
| 135 | 0 | 98 | 99 |
| 136 | 79 | 98 | 97 |
| 137 | 87 | 94 | 99 |
| 138 | 99 | 83 | 100 |
| 139 | 97 | 47 | 96 |
| 140 | 0 | 100 | 100 |
| 141 | 97 | 80 | 100 |
| 142 | 96 | 77 | 93 |
| 143 | 98 | 100 | 99 |
| 144 | 99 | 93 | 98 |
| 146 | 0 | 100 | 99 |
| 147 | 0 | 99 | 99 |
| 148 | 100 | — | 100 |
| 149 | 100 | 100 | 98 |
| 150 | 96 | 100 | 97 |
| 151 | 99 | 100 | 96 |
| 152 | 88 | 100 | 94 |
| 153 | 0 | 92 | 93 |
| 154 | 91 | 99 | 94 |
| 155 | 99 | 100 | 99 |
| 156 | 98 | 96 | 99 |
| 157 | 99 | 100 | 99 |
| 158 | 98 | 38 | 99 |
| 159 | 99 | 13 | 99 |
| 160 | 69 | 99 | 97 |
| 161 | 0 | 100 | 99 |
| 162 | 100 | 100 | 100 |
| 163 | 99 | 100 | 100 |
| 164 | 0 | 97 | 99 |
| 165 | 0 | 0 | 33 |
| 166 | 0 | 46 | 99 |
| 167 | 0 | 64 | 68 |

What is claimed is:

1. A compound selected from Formula 1, N-oxides and salts thereof, 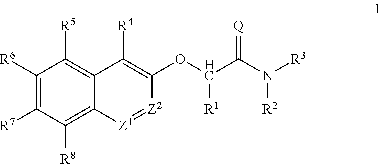
wherein
Q is O or S;
Z¹ and Z² are each independently CR⁹ or N;
R¹ is C₁-C₂ alkyl, C₂-C₄ alkenyl, C₂-C₄ alkynyl, C₁-C₄ haloalkyl, C₂-C₄ haloalkenyl, C₂-C₄ haloalkynyl, C₃-C₄ halocycloalkyl, C₄-C₅ cycloalkylalkyl, C₂-C₄ alkoxyalkyl, C₂-C₄ alkylthioalkyl, C₂-C₄ alkylsulfinylalkyl, C₂-C₄ alkylsulfonylalkyl, C₂-C₄ cyanoalkyl, C₂-C₆ alkylcarbonyl, C₂-C₆ alkoxycarbonyl, C₁-C₄ alkoxy or C₁-C₄ haloalkoxy;
R² is hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₂-C₆ cyanoalkyl, C₂-C₆ alkoxyalkyl, C₃-C₈ alkoxyalkoxyalkyl or benzyloxy(C₂-C₃ alkyl);
R³ is C₁-C₈ alkyl, C₂-C₈ alkenyl or C₂-C₈ alkynyl, each optionally substituted with substituents independently selected from halogen, hydroxy, cyano, nitro, amino, C(═O)OH, C(═O)NH₂, C(═O)R¹⁰, C(═O)OR¹¹, C(═O)NR¹²R¹³, OC(═O)R¹⁰, SC(═O)R¹⁰, OC(═O)OR¹¹, OC(═O)NR¹²R¹³, N(R¹²)C(═O)R¹⁰, N(R¹²)C(═O)OR¹¹, N(R¹²)C(═O)NR¹²R¹³, OSO₂R¹⁴, OSO₂NR¹²R¹³, NR¹²SO²R¹⁴, NR¹²SO₂NR¹²R¹³, OR¹⁵, NR¹²R¹³, S(O)ₙR¹⁴, SO₂NR¹²R¹³, P(═O)(R¹⁷)₂, OP(═O)(R¹⁷)₂, Si(R¹⁸)₃, C(═NNR¹²R¹³)R¹⁹, N═CR¹⁹NR¹²R¹³, CH═NR²¹ and —CHO(CH₂); or
R³ is NR¹²R¹³; or
R³ is a 3-, 4-, 5- or 6-membered saturated carbocyclic ring optionally substituted with up to 5 substituents independently selected from R²⁰; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR¹⁶)_{q}, the heterocyclic ring optionally substituted with up to 5 substituents independently selected from R²⁰ on carbon atom ring members and R^{20a} on nitrogen atom ring members;
R⁴, R⁵, R⁷ and R⁸ are each independently selected from hydrogen, halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylamino, C₂-C₆ dialkylamino, C₂-C₆ haloalkylamino, C₂-C₆ halodialkylamino and C₂-C₆ alkylcarbonylamino;
R⁶ is halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₆ haloalkylamino, C₂-C₆ halodialkylamino or C₂-C₆ alkylcarbonylamino;
each R⁹ is independently selected from hydrogen, halogen, cyano, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₃-C₆ cycloalkenyl, C₂-C₆ alkoxyalkyl, C₂-C₆ alkylthioalkyl, C₂-C₆ alkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ alkoxy, C₁-C₆ haloalkoxy, C₂-C₆ alkoxyalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino and C₂-C₆ alkylcarbonylamino;
each R¹⁰ is independently selected from hydrogen, C₁-C₆ alkyl, C₁-C₆ haloalkyl, C₃-C₆ cycloalkyl and C₃-C₆ halocycloalkyl;
each R¹¹ is independently selected from C₁-C₆ alkyl, C₁-C₆ haloalkyl, C₃-C₆ cycloalkyl and C₃-C₆ halocycloalkyl;
each R¹² is independently selected from hydrogen, CHO, C₁-C₆ alkyl, C₁-C₆ haloalkyl and C₂-C₆ alkylcarbonyl;
each R¹³ is independently selected from CHO, C₁-C₆ alkyl, C₁-C₆ haloalkyl and C₂-C₆ alkylcarbonyl;
each R¹⁴ is independently C₁-C₆ alkyl or C₁-C₆ haloalkyl;
each R¹⁵ is independently selected from CHO, C₁-C₆ alkyl, C₁-C₆ haloalkyl and C₂-C₆ alkoxyalkyl;
each R¹⁶ and R¹⁹ is independently hydrogen or C₁-C₃ alkyl;
each R¹⁷ is independently C₁-C₆ alkyl or C₁-C₆ alkoxy;
each R¹⁸ is independently C₁-C₆ alkyl;
each R²⁰ is independently selected from halogen, cyano, C₁-C₆ alkyl, C₁-C₆ haloalkyl, C₂-C₆ alkylcarbonyl, C₁-C₆ alkoxy, C₁-C₆ haloalkoxy and C₁-C₆ alkylthio;
each R^{20a} is independently selected from cyano, C₁-C₆ alkyl, C₁-C₆ haloalkyl and C₂-C₆ alkylcarbonyl;
each R²¹ is a 5-membered unsaturated heterocyclic ring containing 2-4 carbon atoms and 1-3 nitrogen atoms as ring members, wherein the heterocyclic ring is optionally substituted with up to 2 substituents independently selected from R²⁰ on carbon atom ring members and R^{20a} on nitrogen atom ring members;
each n is independently 0, 1 or 2; and
p and q are independently 0, 1 or 2 in each instance of S(═O)ₚ(═NR¹⁶)_{q}, provided that the sum of p and q is 0, 1 or 2;
provided that
the compound of Formula 1 is other than 2-[(7-methoxy-2-naphthalenyl)oxy]-N-(2-propen-1-yl)-propanamide.

2. A compound of claim 1 wherein:
Q is O;
Z² is CR⁹
R¹ is C₁-C₂ alkyl or C₁-C₄ alkoxy;
R² is hydrogen;
R³ is C₁-C₈ alkyl or C₂-C₈ alkynyl, each optionally substituted with up to 3 substituents independently selected from cyano, hydroxy, OR¹⁵ and CHO;
R⁴, R⁵, R⁷, R⁸ and R⁹ are each independently selected from hydrogen and C₁-C₆ alkyl; and
R⁶ is halogen, C₁-C₂ alkoxy, C₁-C₂ haloalkoxy or C₂-C₃ alkynyl.

3. A compound of claim 2 wherein:
Z¹ is CR⁹;
R¹ is methyl, ethyl or methoxy;
R³ is C₁-C₈ alkyl optionally substituted with cyano or OR¹⁵;
R⁴, R⁵, R⁷, R⁸ and R⁹ are each independently selected from hydrogen, methyl and ethyl; and
R⁶ is halogen, C₁-C₂ alkoxy or C₂-C₃ alkynyl.

4. A compound of claim 2 wherein:
Z¹ is CR⁹;
R¹ is methyl, ethyl or methoxy;
R³ is C₃-C₈ alkynyl;
R⁴, R⁵, R⁷, R⁸ and R⁹ are each independently selected from hydrogen, methyl and ethyl; and
R⁶ is halogen, C₁-C₂ alkoxy or C₂-C₃ alkynyl.

5. A compound of claim 3 wherein:
R³ is C(CH₃)₂CN, C(CH₃)₂CH₂OCH₃, C(CH₃)₂CH₂OCH₂OCH₃ or C(CH₃)₃; and
R⁶ is bromo, methoxy, ethoxy, C≡CH or C≡CCH₃.

6. A compound of claim 4 wherein:
R³ is C(CH₃)₂C≡CH or C(CH₃)₂C≡CCH₃; and
R⁶ is bromo, methoxy, ethoxy, C≡CH or C≡CCH₃.

7. The compound of claim 1 which is selected from the group:
2-[(7-bromo-2-naphthalenyl)oxy]-N-(2-methoxy-1,1-dimethylethyl)butanamide;
2-[(7-bromo-2-naphthalenyl)oxy]-N-[2-(methoxymethoxy)-1,1-dimethylethyl]butanamide;
2-[(6-iodo-3-quinolinyl)oxy]-N-(2-methoxy-1,1-dimethylethyl)butanamide;
N-(1,1-dimethylethyl)-2-[(6-iodo-3-quinolinyl)oxy]butanamide;
N-(1,1-dimethylethyl)-2-[(7-methoxy-2-naphthalenyl)oxy]butanamide;
2-[(7-bromo-2-naphthalenyl)oxy]-N-(1,1-dimethylethyl)butanamide;
2-[(7-bromo-2-naphthalenyl)oxy]-N-[2-[[(dimethylamino)methylene]amino]-1,1-dimethylethyl]butanamide;
2-[(7-bromo-2-naphthalenyl)oxy]-N-(2-methoxy-1,1-dimethylethyl)propanamide;
2-[(7-bromo-2-naphthalenyl)oxy]-N-(2-fluoro-1,1-dimethylethyl)butanamide;
2-[(7-ethynyl-2-naphthalenyl)oxy]-N-(2-methoxy-1,1-dimethylethyl)butanamide;
2-[(7-ethynyl-2-naphthalenyl)oxy]-2-methoxy-N-(2-methoxy-1,1-dimethylethyl)acetamide;
2-[(7-ethynyl-2-naphthalenyl)oxy]-N-[2-(methoxymethoxy)-1,1-dimethylethyl]butanamide;
N-(1-cyano-1-methylethyl)-2-[(7-ethynyl-2-naphthalenyl)oxy]butanamide;
N-(1,1-dimethyl-2-butyn-1-yl)-2-[(7-ethynyl-2-naphthalenyl)oxy]butanamide;
N-(1,1-dimethylethyl)-2-[(7-ethynyl-2-naphthalenyl)oxy]butanamide;
2-[(7-bromo-2-naphthalenyl)oxy]-2-methoxy-N-(2-methoxy-1,1-dimethylethyl)acetamide;
N-(2-methoxy-1,1-dimethylethyl)-2-[[7-(1-propyn-1-yl)-2-naphthalenyl]oxy]butanamide;
2-methoxy-N-(2-methoxy-1,1-dimethylethyl)-2-[[7-(propyn-1-yl)-2-naphthalenyl]oxy]acetamide;
N-(1,1-dimethylethyl)-2-[(7-ethoxy-2-naphthalenyl)oxy]butanamide;
N-(1,1-dimethyl-2-propyn-1-yl)-2-[(7-ethoxy-2-naphthalenyl)oxy]butanamide; and
N-(1,1-dimethyl-2-propyn-1-yl)-2-[(7-ethoxy-2-naphthalenyl)oxy]-2-methoxyacetamide.

8. A fungicidal composition comprising (a) a compound of claim 1; and (b) at least one other fungicide.

9. A fungicidal composition comprising (a) a compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.
